(19) United States
(12) Patent Application Publication
(10) Pub. No.: US 2011/0281812 A1
(43) Pub. Date: Nov. 17, 2011

(54) Compounds

(76) Inventors: Sulejman Alihodzic, Triangle Park, NC (US); Antun Hutinec, Triangle Park, NC (US); Sanja Kostrun, Triangle Park, NC (US); Maja Matanovic Skugor, Triangle Park, NC (US); Ivana Palej Jakopovic, Triangle Park, NC (US)

(21) Appl. No.: 13/146,233

(22) PCT Filed: Jan. 28, 2010
(86) PCT No.: PCT/EP10/50968

§ 371 Date: Jul. 26, 2011

Related U.S. Application Data
(60) Provisional application No. 61/228,362, filed on Jul. 24, 2009.
(60) Provisional application No. 61/148,626, filed on Jan. 30, 2009.

(51) Int. Cl. A61K 31/7048; A61P 11/00; A61P 11/08; A61P 17/00; A61P 19/02; A61P 1/00; A61P 9/10; A61P 29/00; C07H 17/08; A61P 11/06

(52) U.S. Cl. 514/29; 536/7.4

(57) ABSTRACT

2′-O-substituted 14- or 15-membered azalide macrolides of Formula (I)

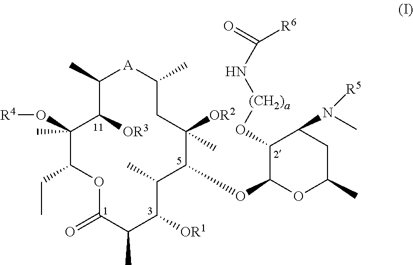

wherein R⁶ represents
(i) —C₁₋₈alkyl, unsubstituted or substituted at the terminal carbon atom by a group selected from hydroxy, —C₁₋₃alkoxy and —C(O)OC₁₋₃alkyl, or when
   —C₁₋₈alkyl is branched, substitution can alternatively be by a hydroxyl group at each of two terminal carbon atoms,
(ii) —CH(NH₂)C₁₋₄alkyl, wherein the —C₁₋₄alkyl group may be optionally interrupted by a heteroatom selected from O, S and N,
(iii) —CH₂N(R⁷)(R⁸), wherein R⁷ and R⁸ each independently represent H or
   —C₁₋₃alkyl provided that R⁷ and R⁸ cannot both simultaneously represent H,
(iv) a 4-6-membered heterocyclic ring containing up to 2 heteroatoms independently selected from O, S and N, wherein the heterocyclic ring is unsubstituted or substituted by —C₁₋₃alkyl,
(v) 5-6 membered heteroaromatic ring, unsubstituted or substituted by one to three groups independently selected from halo, hydroxyl, —C₁₋₃alkyl, —C₁₋₃alkoxy, —CF₃, —OCF₃ and —NH₂,
(vi) —CH(NH₂)CH₂-aryl wherein the aryl group may be unsubstituted or substituted by one or two substituents independently selected from —C₁₋₃alkyl, —C₁₋₃alkoxy and hydroxyl,
(vii) —C₃₋₇cycloalkyl, or
(viii) phenyl unsubstituted or substituted by one or two groups independently selected from halo, hydroxyl, —C₁₋₃alkyl, —C₁₋₃alkoxy, —CF₃, —OCF₃ and —NH₂,
   or salts thereof, useful in the treatment of neutrophil dominated inflammatory diseases, especially in the treatment of neutrophil dominated inflammatory diseases resulting from neutrophilic infiltration and/or diseases associated with altered cellular functionality of neutrophils, to methods for their preparation, to their use as therapeutic agents, and to salts thereof.

### TECHNICAL FIELD

The present invention relates to 2′-O-substituted 14-membered macrolides and 15-membered azalide macrolides useful in the treatment of inflammatory diseases. More particularly, the invention relates to 2′-O-substituted 14-membered macrolides and 15-membered azalide macrolides useful in the treatment of neutrophil dominated inflammatory diseases, especially in the treatment of neutrophil dominated inflammatory diseases resulting from neutrophilic infiltration and/or diseases associated with altered cellular functionality of neutrophils, to methods for their preparation, to their use as therapeutic agents, and to salts thereof.

### BACKGROUND

Inflammation is the final common pathway of various insults, such as infection, trauma, and allergies to the human body. It is characterized by activation of the immune system with recruitment and activation of inflammatory cells and production of pro-inflammatory mediators.

Most inflammatory diseases are characterized by enhanced accumulation of differing proportions of inflammatory cells, including monocytes/macrophages, granulocytes, plasma cells, lymphocytes and platelets. Along with tissue endothelial cells and fibroblasts, these inflammatory cells release a complex array of lipids, growth factors, cytokines and destructive enzymes that cause local tissue damage.

One form of inflammatory response is neutrophilic inflammation which is characterized by infiltration of the inflamed tissue by neutrophilic polymorphonuclear leukocytes (PMN, i.e. neutrophils), which are a major component of host defence. Neutrophils are activated by a great variety of stimuli and are involved in a number of clinical conditions and diseases where they play a pivotal role. Such diseases may be classified according to the major neutrophil-activating event (Table 3, page 638 of V. Witko-Sarsat et al., Laboratory Investigation (2000) 80(5), 617-653) Tissue infection by extracellular bacteria represents the prototype of this inflammatory response. On the other hand, various non-infectious diseases are characterized by extravascular recruitment of neutrophils. These non-infectious inflammatory diseases may be the result of an intermittent resurgence (e.g. flare in autoimmune diseases such as rheumatoid arthritis), or continuous generation (e.g. chronic obstructive pulmonary disease (COPD)) of inflammatory signals arising from underlying immune dysfunction. Non-infectious inflammatory diseases include chronic obstructive pulmonary disease (COPD), cystic fibrosis (CF), diffuse panbronchiolitis (DPB), bronchiolitis obliterans (BOS), bronchitis, bronchiectasis, emphysema, adult respiratory distress syndrome (ARDS, known also as acute respiratory distress syndrome or respiratory distress syndrome, RDS), as well as glomerulonephritis, rheumatoid arthritis, gouty arthritis, ulcerative colitis, certain dermatoses such as psoriasis and vasculitis. In these conditions neutrophils are thought to play a crucial role in the development of tissue injury which, when persistent, can lead to the irreversible destruction of the normal tissue architecture with consequent organ dysfunction. Consequently, correlation between neutrophil number in sputum or bronchoalveolar lavage fluid and disease severity and decline in lung function is demonstrated in patients with chronic obstructive pulmonary disease (Di Stefano et al., Am J Respir Crit Care Med. (1998), 158(4): 1277-1285), cystic fibrosis (Sagel S D et al., J Pediatr. (2002), 141(6): 811-817), diffuse panbronchiolitis (Yanagihara K et al., Int J Antimicrob Agents. (2001), 18 Suppl 1: S83-87), bronchiolitis obliterans (Devouassoux G et al., Transpl Immunol. (2002), 10(4): 303-310), bronchitis (Thompson A B et al., Am Rev Respir Dis. (1989), 140(6): 1527-1537), bronchiectasis (Sepper R et al., Chest (1995), 107(6): 1641-1647), adult respiratory distress syndrome (Weiland J E et al., Am Rev Respir Dis. (1986), 133(2): 218-225), to name a few. In addition, there is increasing evidence of neutrophil inflammation in asthmatics, particularly in patients with severe disease and patients who smoke (Jatakanon A et al., Am J Respir Crit Care Med. (1999), 160: 1532-1539; Chalmers G W et al., Chest (2001), 120: 1917-1922). Evidence of the importance of neutrophils in several lung diseases has prompted a search for drugs that inhibit neutrophilic infiltration into lungs and consequent inflammation (reviewed in Barnes P J, J Allergy Clin Immunol. (2007), 119(5): 1055-1062).

### SUMMARY OF THE INVENTION

The present invention relates to 2′-O-substituted 14-membered macrolides and 15-membered azalide macrolides represented by Formula (I):

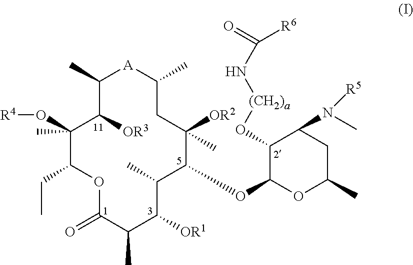

A represents a bivalent radical —C(O)—, —N(R⁹)CH₂—, —CH₂N(R⁹)—, —CH(NR¹⁰R¹¹)—, —C(═NR¹²)—, or —CH(OH)—;
R¹ represents a α-L-cladinosyl group of Formula (a)

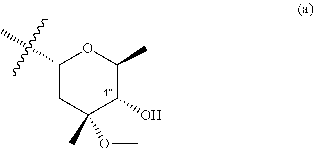

R² represents H or —CH₃;
R³ represents H or —C(O)C₁₋₃alkyl; or R³ and R⁴ taken together with the intervening atoms form a cyclic carbonate group of Formula (b):

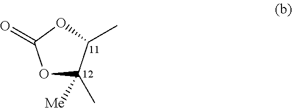

R⁴ represents H; or R³ and R⁴ taken together with the intervening atoms form a cyclic carbonate group of Formula (b);
R⁵ represents H, —C₁₋₄alkyl or —C(O)C₁₋₃alkyl;
R⁶ represents
(i) —C₁₋₈alkyl, unsubstituted or substituted at the terminal carbon atom by a group selected from hydroxy, —C₁₋₃alkoxy and —C(O)OC₁₋₃alkyl, or when —C₁₋₈alkyl is branched, substitution can alternatively be by a hydroxyl group at each of two terminal carbon atoms,
(ii) —CH(NH₂)C₁₋₄alkyl, wherein the —C₁₋₄alkyl group may be optionally interrupted by a heteroatom selected from O, S and N,
(iii) —CH₂N(R⁷)(R⁸), wherein R⁷ and R⁸ each independently represent H or —C₁₋₃alkyl provided that R⁷ and R⁸ cannot both simultaneously represent H,
(iv) a 4-6-membered heterocyclic ring containing up to 2 heteroatoms independently selected from O, S and N, wherein the heterocyclic ring is unsubstituted or substituted by —C₁₋₃alkyl,
(v) 5-6 membered heteroaromatic ring, unsubstituted or substituted by one to three groups independently selected from halo, hydroxyl, —C₁₋₃alkyl, —C₁₋₃alkoxy, —CF₃, —OCF₃ and —NH₂,
(vi) —CH(NH₂)CH₂-aryl wherein the aryl group may be unsubstituted or substituted by one or two substituents independently selected from —C₁₋₃alkyl, —C₁₋₃alkoxy and hydroxyl,
(vii) —C₃₋₇cycloalkyl, or
(viii) phenyl unsubstituted or substituted by one or two groups independently selected from halo, hydroxyl, —C₁₋₃alkyl, —C₁₋₃alkoxy, —CF₃, —OCF₃ and —NH₂,
   R⁹ represents H or —C₁₋₄alkyl;
   R¹⁰ and R¹¹ each independently represent H, —C₁₋₆alkyl or —C(O)R⁹;

### R¹² is —OR¹³;

R¹³ is H or —C₁₋₆alkyl, unsubstituted or substituted by one or two substituents independently selected from cyano, —NR¹⁴R¹⁵ and —C₁₋₆alkoxy; or —C₃₋₇cycloalkyl; or —C₃₋₆alkenyl;
R¹⁴ and R¹⁵ are independently H or —C₁₋₆alkyl;
and
a is an integer from 2 to 6;
or a salt thereof.

The present invention also relates to pharmaceutical compositions comprising a compound of Formula (I) or a pharmaceutically acceptable salt thereof.

Furthermore, the present invention also relates to methods of treating neutrophil dominated inflammatory diseases resulting from neutrophilic infiltration and/or diseases associated with altered cellular functionality of neutrophils comprising administration of a therapeutically effective amount of a compound of Formula (I) or a pharmaceutically acceptable salt thereof to a subject in need thereof.

According to another aspect, the invention relates to a compound of Formula (I) or a pharmaceutically acceptable salt thereof for use in human or veterinary medical therapy.

In another aspect, the invention relates to a compound of Formula (I) or a pharmaceutically acceptable salt thereof, for use in the treatment of neutrophil dominated inflammatory diseases resulting from neutrophilic infiltration and/or diseases associated with altered cellular functionality of neutrophils.

In another aspect, the invention relates to the use of a compound of Formula (I) or a pharmaceutically acceptable salt thereof in the manufacture of a medicament for the treatment of neutrophil dominated inflammatory diseases resulting from neutrophilic infiltration and/or diseases associated with altered cellular functionality of neutrophils.

### BRIEF DESCRIPTION OF THE FIGURES

FIG. 1 shows correlation of inhibition of IL-6 production in vitro and inhibition of cell infiltration into BALF in vivo.

### DESCRIPTION OF THE EMBODIMENTS

It will be understood that the present invention covers all combinations of aspects, suitable, convenient and preferred groups described herein.

The term “alkyl” as used herein, refers to a saturated, straight or branched-chain hydrocarbon radical containing the stated number of carbon atoms, for example, —C₁₋₄alkyl contains between one and four carbon atoms. Examples of “—C₁₋₃alkyl” radicals include: methyl, ethyl, propyl and isopropyl. Examples of “—C₁₋₄alkyl” radicals include: methyl, ethyl, propyl, isopropyl, butyl and tert-butyl. Examples of “—C₁₋₈alkyl” radicals include: methyl, ethyl, propyl, isopropyl, butyl, tert-butyl, n-butyl, isobutyl, sec-butyl, n-pentyl, isopentyl, neopentyl, hexyl, heptanyl, octanyl and the like.

The term “—C₃₋₆alkenyl” as used herein refers to a linear or branched hydrocarbon group containing one or more carbon-carbon double bonds and having from 3 to 6 carbon atoms. Examples of such groups include propenyl, butenyl, pentenyl or hexenyl and the like.

The term “alkoxy” as used herein, refers to an —O-alkyl group wherein alkyl is as defined above. Examples of “—C₁₋₃alkoxy” radicals include: methoxy, ethoxy, propoxy and isopropoxy. Examples of “—C₁₋₆alkoxy” radicals include: methoxy, ethoxy, propoxy, isopropoxy, butoxy, tert-butoxy, n-butoxy, isobutoxy, sec-butoxy, n-pentoxy, isopentoxy, neopentoxy, hexoxy and the like.

The term “cycloalkyl” as used herein, refers to a saturated monocyclic hydrocarbon ring containing the stated number of carbon atoms, for example, 3 to 7 carbon atoms. Examples of such groups include cyclopropyl, cyclobutyl, cyclopentyl, cyclohexyl, cycloheptyl and the like.

The term “heterocyclic ring” refers to a 4-6 membered monocyclic ring which may be saturated or partially unsaturated containing 1 to 2 heteroatoms independently selected from oxygen, nitrogen or sulphur. Examples of such monocyclic rings include pyrrolidinyl, azetidinyl, pyrazolidinyl, oxazolidinyl, piperidinyl, piperazinyl, pyranyl, morpholinyl, thiomorpholinyl, thiazolidinyl, oxiranyl, oxetanyl, dioxolanyl, dioxanyl, oxathiolanyl, oxathianyl, dithianyl, dihydrofuranyl, tetrahydrofuranyl, dihydropyranyl, tetrahydropyranyl, tetrahydropyridinyl, tetrahydropyrimidinyl, tetrahydrothiophenyl, tetrahydrothiopyranyl and the like.

The term “heteroaromatic ring” or “heteroaryl ring” as used herein refers to a 5-6 membered monocyclic aromatic ring containing 1 to 3 heteroatoms independently selected from oxygen, nitrogen and sulphur. Examples of such monocyclic aromatic rings include thienyl, furyl, pyrrolyl, furazanyl, triazolyl, imidazolyl, oxazolyl, thiazolyl, oxadiazolyl, isothiazolyl, isoxazolyl, thiadiazolyl, pyranyl, pyrazolyl, pyrimidyl, pyridazinyl, pyrazinyl, pyridyl, triazinyl and the like.

The term “aryl” as used herein refers to a C₆₋₁₀ monocyclic or bicyclic hydrocarbon ring wherein at least one ring is aromatic. Examples of such groups include phenyl, naphthyl, tetrahydronaphthalenyl and the like.

The term “halogen” or “halo” refers to a fluorine, chlorine, bromine or iodine atom.

The term “inert solvent” or “solvent inert to the reaction”, as used herein, refers to a solvent that cannot react with the dissolved compounds including non-polar solvent such as hexane, toluene, diethyl ether, diisopropylether, chloroform, ethyl acetate, THF, dichloromethane; polar aprotic solvents such as acetonitrile, acetone, N,N-dimethylformamide, N,N-dimethylacetamide, dimethyl sulfoxide, pyridine, and polar protic solvents such as lower alcohol, acetic acid, formic acid and water.

The term “lower alcohol”, as used herein, refers to a C₁₋₄alcohol, such as for example, methanol, ethanol, propanol, isopropanol, butanol, t-butanol, and the like.

In one aspect the present invention relates to a compound of Formula (I) or a salt thereof wherein the salt is a pharmaceutically acceptable salt. For a review on suitable salts see Berge et al., J. Pharm. Sci., 66 (1977) 1-19. Suitable pharmaceutically acceptable salts can include acid or base addition salts.

Suitable addition salts are formed from inorganic or organic acids which form non-toxic salts and examples are hydrochloride, hydrobromide, hydroiodide, sulphate, bisulphate, nitrate, phosphate, hydrogen phosphate, acetate, trifluoroacetate, maleate, malate, fumarate, lactate, tartrate, citrate, formate, gluconate, succinate, salicylate, propionate, pyruvate, hexanoate, oxalate, oxaloacetate, trifluoroacetate, saccharate, glutamate, aspartate, benzoate, alkyl or aryl sulphonates (eg methanesulphonate, ethanesulphonate, benzenesulphonate or p-toluenesulphonate) and isethionate. For example, hydrochloride or acetate salt.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compounds of the invention are within the scope of the invention. The salts of compounds of Formula (I) may form solvates (e.g. hydrates) and the invention also includes all such solvates.

In one aspect, compounds of the present invention may be in the form of pharmaceutically acceptable salts, solvates or solvates of salts. In a further aspect, a compound of Formula (I) of the present invention may be in the form of a pharmaceutically acceptable salt.

References hereinafter to “a compound according to the invention” or “compounds of the present invention” include both a compound of Formula (I) (whether in solvated or unsolvated form), or its pharmaceutically acceptable salts (whether in solvated or unsolvated form).

With regard to stereoisomers, the compounds of Formula (I) have more than one asymmetric carbon atom. In the general Formula (I) as drawn, the solid wedge shaped bond indicates that the bond is above the plane of the paper. The broken bond indicates that the bond is below the plane of the paper.

It will be appreciated that the substituents on the macrolide may also have one or more asymmetric carbon atoms. Thus, the compounds of Formula (I) may occur as individual enantiomers or diastereomers, or mixtures thereof including racemic mixtures. All such isomeric forms are included within the present invention, including mixtures thereof.

Separation of diastereoisomers may be achieved by conventional techniques, e.g. by fractional crystallisation, chromatography or H.P.L.C. An individual stereoisomer may also be prepared from a corresponding optically pure intermediate or by resolution, such as H.P.L.C., of the corresponding mixture using a suitable chiral support or by fractional crystallisation of the diastereoisomeric salts formed by reaction of the corresponding mixture with a suitable optically active acid or base, as appropriate.

It will be appreciated that compounds of the invention may exist as geometric isomers (cis/trans or (E)/(Z)). The present invention includes the individual geometric isomers of the compounds of the invention and, where appropriate, mixtures thereof.

The compounds of Formula (I) may be in crystalline or amorphous form. Furthermore, some of the crystalline forms of the compounds of Formula (I) may exist as polymorphs, which are included in the present invention.

In one aspect, the present invention is directed to 2′-O-substituted 14- or 15-membered azalide macrolides of Formula (I) represented by Formula (Ia):

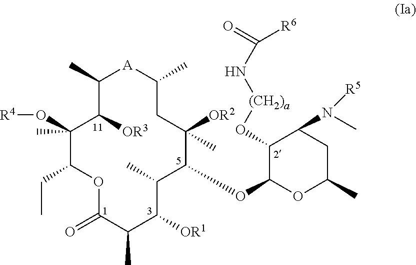

A represents a bivalent radical —C(O)—, —N(R⁹)CH₂—, —CH₂N(R⁹)—, —CH(NR¹⁰R¹¹)—, —C(═NR¹²)—, or —CH(OH)—;

R¹ represents a α-L-cladinosyl group of Formula (a)

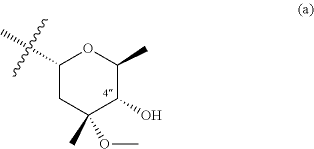

R² represents H or CH₃;
R³ represents H or —C(O)C₁₋₃alkyl; or R³ and R⁴ taken together with the intervening atoms form a cyclic carbonate group of Formula (b):

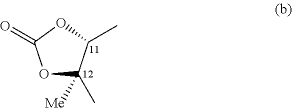

R⁴ represents H; or R³ and R⁴ taken together with the intervening atoms form a cyclic carbonate group of Formula (b);
R⁵ represents H, C₁₋₄alkyl or —C(O)C₁₋₃alkyl;
R⁶ represents
(i) C₁₋₈alkyl, optionally substituted by hydroxy at the terminal carbon atom,
(ii) —CH(NH₂)C₁₋₄alkyl, wherein the C₁₋₄alkyl group may be optionally interrupted by a heteroatom selected from O, S and N,
(iii) —CH₂N(R⁷)(R⁸), wherein R⁷ and R⁸ each independently represent hydrogen or C₁₋₃alkyl provided that R⁷ and R⁸ cannot both simultaneously represent hydrogen,
(iv) a 4-6-membered heterocyclic ring containing up to 2 heteroatoms independently selected from O, S and N, wherein the heterocyclic ring may be optionally substituted by C₁₋₃alkyl,
(v) pyridyl, or
(vi) —CH(NH₂)CH₂-aryl wherein the aryl group may be unsubstituted or substituted by one or two substituents independently selected from C₁₋₃alkyl, C₁₋₃alkoxy and hydroxyl;
   R⁹ represents H or C₁₋₄alkyl;
   R¹⁰ and R¹¹ are each independently H, C₁₋₆alkyl or —C(O)R⁹;

### R¹² is —OR¹³;

R¹³ is H or C₁₋₆alkyl optionally substituted by one or two substituents independently selected from cyano, —NR¹⁴R¹⁵ and C₁₋₆alkoxy; C₃₋₇cycloalkyl; or C₃₋₆alkenyl;
R¹⁴ and R¹⁵ are independently H or C₁₋₆alkyl;
and
a is an integer from 2 to 6;
or a salt thereof.

In one aspect of the invention A is a bivalent radical —N(R⁹)CH₂— wherein R⁹ is —C₁₋₄alkyl. In a further aspect of the invention A is a bivalent radical —N(R⁹)CH₂— wherein R⁹ is methyl.

In one aspect of the invention R² is H.

In one aspect of the invention R³ is H.

In one aspect of the invention R⁴ is H.

In one aspect of the invention R⁵ is —C₁₋₄alkyl. In one aspect of the invention R⁵ is methyl.

In one aspect of the invention R⁶ is —C₁₋₄alkyl. In a further aspect of the invention R⁶ is methyl, ethyl, isopropyl or tert-butyl. In a further aspect R⁶ is —C₁₋₄alkyl substituted at the terminal carbon atom by —C₁₋₃alkoxy, —C(O)OC₁₋₃alkyl or hydroxyl. In a further aspect R⁶ is —C₁₋₄alkyl substituted at the terminal carbon atom by —C₁₋₃alkoxy. In a further aspect R⁶ is methyl substituted at the terminal carbon atom by —C₁₋₃alkoxy.

In one aspect of the invention R⁶ is branched —C₁₋₈alkyl substituted by a hydroxyl group at each of two terminal carbon atoms. For example, a branched —C₁₋₈alkyl with three terminal carbon atoms may be subtituted at two of the three terminal carbon atoms by hydroxyl groups, such as —CH(CH₃)(CH₂OH)(CH₂OH).

In one aspect of the invention R⁶ is —CH(NH₂)C₁₋₄alkyl. In a further aspect of the invention R⁶ is —CH(NH₂)C₁₋₄alkyl wherein the —C₁₋₄alkyl is interrupted by a sulphur.

In one aspect of the invention R⁶ is —CH₂N(R⁷)(R⁸), wherein R⁷ and R⁸ each independently represent H or —C₁₋₃alkyl. In a further aspect of the invention R⁶ is —CH₂N(R⁷)(R⁸), wherein R⁷ and R⁸ each independently represent H, methyl or ethyl.

In one aspect of the invention R⁶ is a 4-6-membered heterocyclic ring containing up to 2 heteroatoms independently selected from O, S and N. In a further aspect of the invention R⁶ is a 5-membered heterocyclic ring containing one heteroatom which is nitrogen. In a further aspect, R⁶ is pyrrolidin-2-yl, optionally N-substituted by methyl. In a further aspect of the invention R⁶ is a 6-membered heterocyclic ring containing one heteroatom which is oxygen.

In one aspect of the invention R⁶ is a 5-6 membered heteroaromatic ring selected from furanyl, pyrazinyl, pyrazolyl, pyridyl, pyrrolyl and imidazolyl. In a further aspect of the invention R⁶ is 5-6 membered heteroaromatic ring substituted by one to three groups independently selected from halo, CH₃, CF₃, and NH₂.

In one aspect of the invention R⁶ is pyridyl.

In one aspect of the invention R⁶ is oxazolyl.

In one aspect of the invention R⁶ is —CH(NH₂)CH₂-aryl. In a further aspect of the invention R⁶ is —CH(NH₂)CH₂-phenyl.

In one aspect of the invention R⁶ is phenyl substituted by one or two groups independently selected from halo, hydroxyl and NH₂.

In one aspect of the invention R⁶ is C₃₋₇cycloalkyl. In a further aspect of the invention, R₆ is cyclobutyl.

In one aspect of the invention integer a is 3.

In one aspect, the present invention is directed to compound of Formula (I) wherein A is the bivalent radical —N(CH₃)CH₂—, R₂ is H, R₃ is H, R₄ is H, R₅ is methyl, R₆ is C₁₋₄alkyl substituted at the terminal carbon atom by —O—C₁₋₃alkyl and a is 3.

In one aspect, the present invention comprises a compound of Formula (I) selected from:
2′-O-[3-(acetylamino)propyl]-9-deoxo-9a-methyl-9a-aza-9a-homoerythromycin A,
2′-O-[3-(propanoylamino)propyl]-9-deoxo-9a-methyl-9a-aza-9a-homoerythromycin A,
2′-O-{3-[(2-methylpropanoyl)amino]propyl}-9-deoxo-9a-methyl-9a-aza-9a-homoerythromycin A,
2′-O-{3-[(2,2-dimethylpropanoyl)amino]propyl}-9-deoxo-9a-methyl-9a-aza-9a-homoerythromycin A,
2′-O-{3-[(N,N-diethylglycyl)amino]propyl}-9-deoxo-9a-methyl-9a-aza-9a-homoerythromycin A,
2′-O-{3-[(4-pyridinylcarbonyl)amino]propyl}-9-deoxo-9a-methyl-9a-aza-9a-homoerythromycin A,
2′-O-{3-[(N,N-dimethylglycyl)amino]propyl}-9-deoxo-9a-methyl-9a-aza-9a-homoerythromycin A,
2′-O-{3-[(N-methylglycyl)amino]propyl}-9-deoxo-9a-methyl-9a-aza-9a-homoerythromycin A,
2′-O-[3-(L-prolylamino)propyl]-9-deoxo-9a-methyl-9a-aza-9a-homoerythromycin A,
2′-O-[3-(L-phenylalanylamino)propyl]-9-deoxo-9a-methyl-9a-aza-9a-homoerythromycin A,
2′-O-[3-(L-isoleucylamino)propyl]-9-deoxo-9a-methyl-9a-aza-9a-homoerythromycin A, and
2′-O-[3-(L-methionylamino)propyl]-9-deoxo-9a-methyl-9a-aza-9a-homoerythromycin A,
2′-O-(3-{[4-(methyloxy)-4-oxobutanoyl]amino}propyl)-9-deoxo-9a-methyl-9a-aza-9a-homoerythromycin A,
2′-O-(3-{[5-(methyloxy)-5-oxopentanoyl]amino}propyl)-9-deoxo-9a-methyl-9a-aza-9a-homoerythromycin A,
2′-O-{3-[(cyclobutylcarbonyl)amino]propyl}-9-deoxo-9a-methyl-9a-aza-9a-homoerythromycin A,
2′-O-{3-[(methoxyacetyl)amino]propyl}-9-deoxo-9a-methyl-9a-aza-9a-homoerythromycin A,
2′-O-{3-[(3-furanylcarbonyl)amino]propyl}-9-deoxo-9a-methyl-9a-aza-9a-homoerythromycin A,
2′-O-(3-{[(5-methyl-2-pyrazinyl)carbonyl]amino}propyl)-9-deoxo-9a-methyl-9a-aza-9a-homoerythromycin A,
2′-O-(3-{[(1,3,5-trimethyl-1H-pyrazol-4-yl)carbonyl]amino}propyl)-9-deoxo-9a-methyl-9a-aza-9a-homoerythromycin A,
2′-O-{3-[(3-pyridinylcarbonyl)amino]propyl}-9-deoxo-9a-methyl-9a-aza-9a-homoerythromycin A,
2′-O-{3-[(ethyloxyacetyl)amino]propyl}-9-deoxo-9a-methyl-9a-aza-9a-homoerythromycin A,
2′-O-(3-{[3-hydroxy-2-(hydroxymethyl)-2-methylpropanoyl]amino}propyl)-9-deoxo-9a-methyl-9a-aza-9a-homoerythromycin A,
2′-O-{3-[(2-pyrazinylcarbonyl)amino]propyl}-9-deoxo-9a-methyl-9a-aza-9a-homoerythromycin A,
2′-O-(3-{[(2,5-dimethyl-3-furanyl)carbonyl]amino}propyl)-9-deoxo-9a-methyl-9a-aza-9a-homoerythromycin A,
2′-O-{3-[(tetrahydro-2H-pyran-4-ylcarbonyl)amino]propyl}-9-deoxo-9a-methyl-9a-aza-9a-homoerythromycin A,
2′-O-(3-{[(1,2,5-trimethyl-1H-pyrrol-3-yl)carbonyl]amino}propyl)-9-deoxo-9a-methyl-9a-aza-9a-homoerythromycin A,
2′-O-(3-{[(1-methyl-1H-imidazol-5-yl)carbonyl]amino}propyl)-9-deoxo-9a-methyl-9a-aza-9a-homoerythromycin A,
2′-O-[3-({[1-methyl-3-(trifluoromethyl)-1H-pyrazol-4-yl]carbonyl}amino)propyl]-9-deoxo-9a-methyl-9a-aza-9a-homoerythromycin A,
2′-O-(3-{[(4-chlorophenyl)carbonyl]amino}propyl)-9-deoxo-9a-methyl-9a-aza-9a-homoerythromycin A,
2′-O-{3-[(hydroxyacetyl)amino]propyl}-9-deoxo-9a-methyl-9a-aza-9a-homoerythromycin A,
2′-O-{3-[(2-pyridinylcarbonyl)amino]propyl}-9-deoxo-9a-methyl-9a-aza-9a-homoerythromycin A,
2′-O-{3-[(2,5-dihydroxyphenylcarbonyl)amino]propyl}-9-deoxo-9a-methyl-9a-aza-9a-homoerythromycin A,
2′-O-(3-{[(4-amino-2-hydroxyphenyl)carbonyl]amino}propyl)-9-deoxo-9a-methyl-9a-aza-9a-homoerythromycin A,
2′-O-(3-{[(2-chloro-3-pyridinyl)carbonyl]amino}propyl)-9-deoxo-9a-methyl-9a-aza-9a-homoerythromycin A,
2′-O-(3-{[(2-chloro-6-methyl-3-pyridinyl)carbonyl]amino}propyl)-9-deoxo-9a-methyl-9a-aza-9a-homoerythromycin A,
2′-O-(3-{[(5-amino-3-pyridinyl)carbonyl]amino}propyl)-9-deoxo-9a-methyl-9a-aza-9a-homoerythromycin A,
2′-O-(3-{[(3-amino-2-pyrazinyl)carbonyl]amino}propyl)-9-deoxo-9a-methyl-9a-aza-9a-homoerythromycin A,
2′-O-(3-{[(5-chloro-1,3-dimethyl-1H-pyrazol-4-yl)carbonyl]amino}propyl)-9-deoxo-9a-methyl-9a-aza-9a-homoerythromycin A,
2′-O-(3-{[(2,5-dimethyl-1,3-oxazol-4-yl)carbonyl]amino}propyl)-9-deoxo-9a-methyl-9a-aza-9a-homoerythromycin A,
2′-O-{3-[(1-methyl-L-prolyl)amino]propyl}-9-deoxo-9a-methyl-9a-aza-9a-homoerythromycin A,
2′-O-{3-[(3-methylbutanoyl)amino]propyl}-9-deoxo-9a-methyl-9a-aza-9a-homoerythromycin A,
   or a salt thereof.

In one aspect, the present invention comprises a compound of Formula (I) which is 2′-O-{3-[(methoxyacetyl)amino]propyl}-9-deoxo-9a-methyl-9a-aza-9a-homoerythromycin A.

In one aspect, the present invention comprises a compound of Formula (I) which is 2′-O-{3-[(methoxyacetyl)amino]propyl}-9-deoxo-9a-methyl-9a-aza-9a-homoerythromycin A, or a salt thereof.

In one aspect, the present invention comprises a compound of Formula (I) which is 2′-O-{3-[(methoxyacetyl)amino]propyl}-9-deoxo-9a-methyl-9a-aza-9a-homoerythromycin A, or a pharmaceutically acceptable salt thereof.

In one aspect, the present invention comprises a compound of Formula (I) which is 2′-O-{3-[(ethyloxyacetyl)amino]propyl}-9-deoxo-9a-methyl-9a-aza-9a-homoerythromycin A.

In one aspect, the present invention comprises a compound of Formula (I) which is 2′-O-{3-[(ethyloxyacetyl)amino]propyl}-9-deoxo-9a-methyl-9a-aza-9a-homoerythromycin A, or a salt thereof.

In one aspect, the present invention comprises a compound of Formula (I) which is 2′-O-{3-[(ethyloxyacetyl)amino]propyl}-9-deoxo-9a-methyl-9a-aza-9a-homoerythromycin A, or a pharmaceutically acceptable salt thereof.

Compounds of the present invention inhibit infiltration of neutrophils into inflamed lung tissue (as demonstrated hereinafter). Therefore these compounds have potential utility in acute and chronic treatment of inflammatory pathologies, especially of those pathologies associated with extensive neutrophil infiltration into the lung tissue, for example chronic obstructive pulmonary disease (COPD), cystic fibrosis (CF), diffuse panbronchiolitis (DPB), bronchiolitis obliterans (BOS), bronchitis, bronchiectasis, adult respiratory distress syndrome (ARDS known also as acute respiratory distress syndrome or respiratory distress syndrome, RDS), severe or steroid-resistant asthma (Simpson J L et al. (2008) Am J Respir Crit Care Med, 177: 148-155), and emphysema or into the respiratory tract, for example chronic rhinosinusitis (with or without nasal polyposis) (Wallwork B et al. (2006) Laryngoscope, 116: 189-193). In addition, compounds of the present invention can be used for the treatment of other diseases associated with altered cellular functionality of neutrophils, for example rheumatoid arthritis (Kitsis E and, Weissmann G, Clin Orthop Relat Res. (1991), 265: 63-72), gouty arthritis, inflammatory bowel diseases (such as ulcerative colitis and Chron's disease), glomerulonephritis (Heinzelmann M et al., Am J Kidney Dis. (1999), 34(2): 384-399), damage from ischemic reperfusion (Kaminski K A et al., Int J Cardiol. (2002), 86(1): 41-59), atherosclerosis (Henriksen P A and Sallenave J M. Int J Biochem Cell (2008) in press), dermatoses such as psoriasis (Terui T et al., Exp Dermatol. (2000), 9(1): 1-10) and vasculitis, systemic lupus erythematodes (SLE), systemic inflammatory response syndrome (SIRS), sepsis, ischemia-reperfusion injury, rosacea, periodontitis, gingival hyperplasia and prostatitis syndrome.

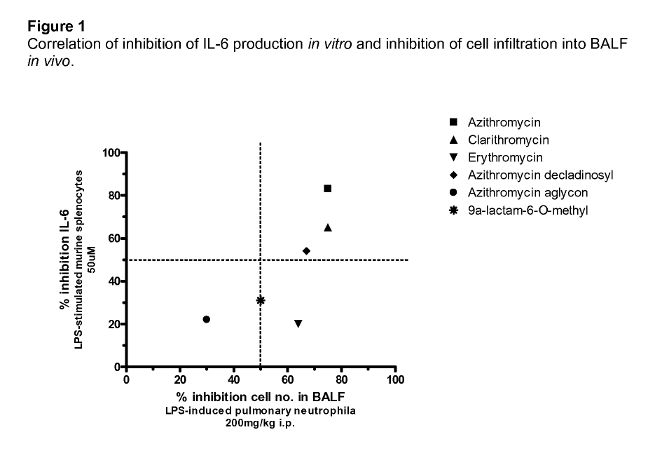

The induction of lung neutrophil infiltration in rodents by the local application of bacterial lipopolysaccharide (LPS) is widely used as a test model for neutrophilic infiltration of human lungs during pulmonary inflammatory disease. We have observed a correlation between the inhibitory activity of compounds on cell infiltration into broncho-alveolar lung fluid (BALF) of mice treated intranasally with LPS and their inhibition of interleukin-6 (IL-6) production by LPS-stimulated mouse splenocytes in vitro (FIG. 1). Therefore, inhibition of IL-6 production in LPS-stimulated murine spleen cells may be a suitable in-vitro model (biomarker) for the in-vivo activity of compounds in treating inflammatory diseases resulting from neutrophilic infiltration and/or diseases associated with altered cellular functionality of neutrophils.

“Treating” or “treatment” of neutrophil dominated inflammatory diseases, especially those resulting from neutrophilic infiltration and/or diseases associated with altered cellular functionality of neutrophils means the alleviation of the symptoms and/or retardation of progression of the disease, and may include the suppression of symptom recurrence in an asymptomatic patient.

Inflammatory diseases resulting from neutrophilic infiltration and/or diseases associated with altered cellular functionality of neutrophils include chronic obstructive pulmonary disease (COPD), cystic fibrosis (CF), diffuse panbronchiolitis (DPB), bronchiolitis obliterans (BOS), bronchitis, bronchiectasis, adult respiratory distress syndrome (ARDS), severe or steroid-resistant asthma, emphysema, chronic rhinosinusitis (with or without nasal polyposis), rheumatoid arthritis, gouty arthritis, inflammatory bowel disease (ulcerative colitis and Chron's disease), glomerulonephritis, damage from ischemic reperfusion, atherosclerosis, dermatoses such as psoriasis and vasculitis, systemic lupus erythematosus (SLE), systemic inflammatory response syndrome (SIRS), sepsis, ischemia-reperfusion injury, rosacea, periodontitis, gingival hyperplasia and prostatitis syndrome.

In one aspect, the present invention provides a method of treating chronic obstructive pulmonary disease (COPD), cystic fibrosis (CF), diffuse panbronchiolitis (DPB), bronchiolitis obliterans (BOS), bronchitis, bronchiectasis, adult respiratory distress syndrome (ARDS), severe or steroid-resistant asthma, emphysema, chronic rhinosinusitis (with or without nasal polyposis), rheumatoid arthritis, gouty arthritis, inflammatory bowel disease (ulcerative colitis and Chron's disease), glomerulonephritis, damage from ischemic reperfusion, atherosclerosis, dermatoses such as psoriasis and vasculitis, systemic lupus erythematosus (SLE), systemic inflammatory response syndrome (SIRS), sepsis, ischemia-reperfusion injury, rosacea, periodontitis, gingival hyperplasia and prostatitis syndrome in a subject in need of such treatment comprising administering to the subject a therapeutically effective amount of a compound of formula (I) or a pharmaceutically acceptable salt thereof.

In one aspect, the present invention provides a method of treating chronic obstructive pulmonary disease, cystic fibrosis, diffuse panbronchiolitis, bronchiolitis obliterans, bronchitis, bronchiectasis, adult respiratory distress syndrome, severe or steroid-resistant asthma, emphysema and chronic rhinosinusitis (with or without nasal polyposis) in a subject in need of such treatment comprising administering to the subject a therapeutically effective amount of a compound of formula (I) or a pharmaceutically acceptable salt thereof.

In one aspect, the present invention provides a method of treating chronic obstructive pulmonary disease in a subject in need of such treatment comprising administering to the subject a therapeutically effective amount of a compound of formula (I) or a pharmaceutically acceptable salt thereof.

In a further aspect, the present invention provides a method of treating bronchiolitis obliterans in a subject in need of such treatment comprising administering to the subject a therapeutically effective amount of a compound of formula (I) or a pharmaceutically acceptable salt thereof.

In a further aspect, the present invention provides a method of treating severe or steroid-resistant asthma in a subject in need of such treatment comprising administering to the subject a therapeutically effective amount of a compound of formula (I) or a pharmaceutically acceptable salt thereof.

In a further aspect, the present invention provides a method of treating cystic fibrosis in a subject in need of such treatment comprising administering to the subject a therapeutically effective amount of a compound of formula (I) or a pharmaceutically acceptable salt thereof.

In one aspect, the present invention provides a method of treating chronic rhinosinusitis (with or without nasal polyposis) in a subject in need of such treatment comprising administering to the subject a therapeutically effective amount of a compound of formula (I) or a pharmaceutically acceptable salt thereof.

In one aspect, the present invention provides a compound of Formula (I) or a pharmaceutically acceptable salt thereof for use in medical therapy.

In one aspect, the present invention provides a compound of Formula (I) or a pharmaceutically acceptable salt thereof, for use in the treatment of chronic obstructive pulmonary disease (COPD), cystic fibrosis (CF), diffuse panbronchiolitis (DPB), bronchiolitis obliterans (BOS), bronchitis, bronchiectasis, adult respiratory distress syndrome (ARDS), severe or steroid-resistant asthma, emphysema, chronic rhinosinusitis (with or without nasal polyposis), rheumatoid arthritis, gouty arthritis, inflammatory bowel disease (ulcerative colitis and Chron's disease), glomerulonephritis, damage from ischemic reperfusion, atherosclerosis, dermatoses such as psoriasis and vasculitis, systemic lupus erythematosus (SLE), systemic inflammatory response syndrome (SIRS), sepsis, ischemia-reperfusion injury, rosacea, periodontitis, gingival hyperplasia and prostatitis syndrome.

In another aspect, the present invention provides a compound of Formula (I) or a pharmaceutically acceptable salt thereof, for use in the treatment of chronic obstructive pulmonary disease, cystic fibrosis, diffuse panbronchiolitis, bronchiolitis obliterans, bronchitis, bronchiectasis, adult respiratory distress syndrome, severe or steroid-resistant asthma, emphysema and chronic rhinosinusitis (with or without nasal polyposis).

In a further aspect, the present invention provides a compound of Formula (I) or a pharmaceutically acceptable salt thereof, for use in the treatment of chronic obstructive pulmonary disease.

In a further aspect, the present invention provides a compound of Formula (I) or a pharmaceutically acceptable salt thereof, for use in the treatment of bronchiolitis obliterans.

In a further aspect, the present invention provides a compound of Formula (I) or a pharmaceutically acceptable salt thereof, for use in the treatment of severe or steroid-resistant asthma.

In a further aspect, the present invention provides a compound of Formula (I) or a pharmaceutically acceptable salt thereof, for use in the treatment of cystic fibrosis.

In a further aspect, the present invention provides a compound of Formula (I) or a pharmaceutically acceptable salt thereof, for use in the treatment of chronic rhinosinusitis (with or without nasal polyposis).

In a further aspect, the present invention provides the use of a compound of Formula (I) or a pharmaceutically acceptable salt thereof in the manufacture of a medicament for the treatment of chronic obstructive pulmonary disease (COPD), cystic fibrosis (CF), diffuse panbronchiolitis (DPB), bronchiolitis obliterans (BOS), bronchitis, bronchiectasis, adult respiratory distress syndrome (ARDS), severe or steroid-resistant asthma, emphysema, chronic rhinosinusitis (with or without nasal polyposis), rheumatoid arthritis, gouty arthritis, inflammatory bowel disease (ulcerative colitis and Chron's disease), glomerulonephritis, damage from ischemic reperfusion, atherosclerosis, dermatoses such as psoriasis and vasculitis, systemic lupus erythematosus (SLE), systemic inflammatory response syndrome (SIRS), sepsis, ischemia-reperfusion injury, rosacea, periodontitis, gingival hyperplasia and prostatitis syndrome.

In a further aspect of the invention, the present invention provides the use of a compound of Formula (I) or pharmaceutically acceptable salt thereof in the manufacture of a medicament for the treatment of chronic obstructive pulmonary disease, cystic fibrosis, diffuse panbronchiolitis, bronchiolitis obliterans, bronchitis, bronchiectasis, adult respiratory distress syndrome, severe or steroid-resistant asthma, emphysema and chronic rhinosinusitis (with or without nasal polyposis).

In a further aspect, the present invention provides the use of a compound of Formula (I) or pharmaceutically acceptable salt thereof in the manufacture of a medicament for the treatment of chronic obstructive pulmonary disease.

In a further aspect, the present invention provides the use of a compound of Formula (I) or pharmaceutically acceptable salt thereof in the manufacture of a medicament for the treatment of bronchiolitis obliterans.

In a further aspect, the present invention provides the use of a compound of Formula (I) or pharmaceutically acceptable salt thereof in the manufacture of a medicament for the treatment of severe or steroid-resistant asthma.

In a further aspect, the present invention provides the use of a compound of Formula (I) or pharmaceutically acceptable salt thereof in the manufacture of a medicament for the treatment of cystic fibrosis.

In a further aspect, the present invention provides the use of a compound of Formula (I) or pharmaceutically acceptable salt thereof in the manufacture of a medicament for the treatment of chronic rhinosinusitis (with or without nasal polyposis).

The present invention is also directed to pharmaceutical compositions comprising a compound of Formula (I) or a pharmaceutically acceptable salt thereof in an amount effective for therapeutic treatment of chronic obstructive pulmonary disease (COPD), cystic fibrosis (CF), diffuse panbronchiolitis (DPB), bronchiolitis obliterans (BOS), bronchitis, bronchiectasis, adult respiratory distress syndrome (ARDS), severe or steroid-resistant asthma, emphysema, chronic rhinosinusitis (with or without nasal polyposis), rheumatoid arthritis, gouty arthritis, inflammatory bowel disease (ulcerative colitis and Chron's disease), glomerulonephritis, damage from ischemic reperfusion, atherosclerosis, dermatoses such as psoriasis and vasculitis, systemic lupus erythematosus (SLE), systemic inflammatory response syndrome (SIRS), sepsis, ischemia-reperfusion injury, rosacea, periodontitis, gingival hyperplasia and prostatitis syndrome in a subject in need of such treatment.

In another aspect, the present invention is also directed to compositions comprising a compound of Formula (I) or a pharmaceutically acceptable salt thereof in an amount effective for therapeutic treatment of chronic obstructive pulmonary disease, cystic fibrosis, diffuse panbronchiolitis, bronchiolitis obliterans, bronchitis, bronchiectasis, acute respiratory distress syndrome, severe or steroid-resistant asthma, emphysema and chronic rhinosinusitis (with or without nasal polyposis), in a subject in need of such treatment.

The present invention is further related to a pharmaceutical composition for the treatment of chronic obstructive pulmonary disease (COPD), cystic fibrosis (CF), diffuse panbronchiolitis (DPB), bronchiolitis obliterans (BOS), bronchitis, bronchiectasis, adult respiratory distress syndrome (ARDS), severe or steroid-resistant asthma, emphysema, chronic rhinosinusitis (with or without nasal polyposis), rheumatoid arthritis, gouty arthritis, inflammatory bowel disease (ulcerative colitis and Chron's disease), glomerulonephritis, damage from ischemic reperfusion, atherosclerosis, dermatoses such as psoriasis and vasculitis, systemic lupus erythematosus (SLE), systemic inflammatory response syndrome (SIRS), sepsis, ischemia-reperfusion injury, rosacea, periodontitis, gingival hyperplasia and prostatitis syndrome comprising a compound of Formula (I) or a pharmaceutically acceptable salt thereof.

The present invention is further related to a pharmaceutical composition for the treatment of chronic obstructive pulmonary disease, cystic fibrosis, diffuse panbronchiolitis, bronchiolitis obliterans, bronchitis, bronchiectasis, acute respiratory distress syndrome, severe or steroid-resistant asthma, emphysema and chronic rhinosinusitis (with or without nasal polyposis), comprising a compound of Formula (I) or a pharmaceutically acceptable salt thereof.

The benefit to a subject to be treated is either statistically significant or at least perceptible to the subject or to the physician.

“Subject” refers to an animal, in particular a mammal and more particularly to a human or a domestic animal or an animal serving as a model for a disease (e.g., mouse, monkey, etc.). In one aspect, the subject is a human.

A “therapeutically effective amount” means the amount of a compound that, when administered to a subject for treating a neutrophil dominated inflammatory disease resulting from neutrophilic infiltration and/or diseases associated with altered cellular functionality of neutrophils is sufficient to effect such treatment. The “therapeutically effective amount” will vary depending on the disease and its severity and the age, weight, physical condition and responsiveness of the subject to be treated and will ultimately be at the discretion of the attendant physician.

### Pharmaceutical Compositions

While it is possible that, for use in the methods of the invention, a compound of Formula (I) or a pharmaceutically acceptable salt thereof may be administered as the bulk substance, it is preferable to present the active ingredient in a pharmaceutical formulation, for example, wherein the agent is in admixture with at least one pharmaceutically acceptable carrier selected with regard to the intended route of administration and standard pharmaceutical practice.

Accordingly, the present invention provides a pharmaceutical composition comprising a) a compound of Formula (I) or a pharmaceutically acceptable salt thereof and b) one or more pharmaceutically acceptable carriers.

The term “carrier” refers to a diluent, excipient, and/or vehicle with which an active compound is administered. The pharmaceutical compositions of the invention may contain combinations of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition. The choice of pharmaceutical carrier can be selected with regard to the intended route of administration and standard pharmaceutical practice. The pharmaceutical compositions may comprise, in addition to the carrier, any suitable binder(s), lubricant(s), suspending agent(s), coating agent(s), and/or solubilizing agent(s).

The phrase “pharmaceutically acceptable”, as used herein, refers to salts, molecular entities and other ingredients of compositions that are generally physiologically tolerable and do not typically produce untoward reactions when administered to a mammal (e.g., human). Suitably, as used herein, the term “pharmaceutically acceptable” means approved by a regulatory agency of the Federal or a state government or listed in the U.S. Pharmacopoeia or other generally recognized pharmacopoeia for use in mammals, and more particularly in humans.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

The present invention is further related to a pharmaceutical composition for the treatment of a neutrophil dominated inflammatory diseases resulting from neutrophilic infiltration and/or diseases associated with altered cellular functionality of neutrophils comprising a compound of Formula (I) or a pharmaceutically acceptable salt thereof.

The present invention is even further related to a pharmaceutical composition comprising a) 10 to 2000 mg of a compound of Formula (I) or a pharmaceutically acceptable salt thereof, and b) 0.1 to 2 g of one or more pharmaceutically acceptable excipients.

It will be appreciated that pharmaceutical compositions for use in accordance with the present invention may be in the form of oral, parenteral, transdermal, inhalation, sublingual, topical, implant, nasal, or enterally administered (or other mucosally administered) suspensions, solutions, capsules or tablets, which may be formulated in conventional manner using one or more pharmaceutically acceptable carriers or excipients.

In one aspect, the pharmaceutical composition is formulated for oral administration.

The compounds of the invention can be administered for immediate-, delayed-, modified-, sustained-, pulsed- or controlled-release applications.

In one aspect, oral compositions are slow, delayed or positioned release (e.g., enteric especially colonic release) tablets or capsules. This release profile can be achieved, for example, by use of a coating resistant to conditions within the stomach but releasing the contents in the colon or other portion of the GI tract wherein a lesion or inflammation site has been identified. Or a delayed release can be achieved by a coating that is simply slow to disintegrate. Or the two (delayed and positioned release) profiles can be combined in a single formulation by choice of one or more appropriate coatings and other excipients. Such formulations constitute a further feature of the present invention.

Suitable compositions for delayed or positioned release and/or enteric coated oral formulations include tablet formulations film coated with materials that are water resistant, pH sensitive, digested or emulsified by intestinal juices or sloughed off at a slow but regular rate when moistened. Suitable coating materials include, but are not limited to, hydroxypropyl methylcellulose, ethyl cellulose, cellulose acetate phthalate, polyvinyl acetate phthalate, hydroxypropyl methylcellulose phthalate, polymers of metacrylic acid and its esters, and combinations thereof. Plasticizers such as, but not limited to polyethylene glycol, dibutylphthalate, triacetin and castor oil may be used. A pigment may also be used to color the film. Suppositories may be prepared by using carriers like cocoa butter, suppository bases such as Suppocire C, and Suppocire NA50 (supplied by Gattefossé Deutschland GmbH, D-Weil am Rhein, Germany) and other Suppocire type excipients obtained by interesterification of hydrogenated palm oil and palm kernel oil (C₈-C₁₈ triglycerides), esterification of glycerol and specific fatty acids, or polyglycosylated glycerides, and whitepsol (hydrogenated plant oils derivatives with additives). Enemas are formulated by using the appropriate active compound according to the present invention and solvents or excipients for suspensions. Suspensions may be produced by using micronized compounds, and appropriate vehicle containing suspension stabilizing agents, thickeners and emulsifiers like carboxymethylcellulose and salts thereof, polyacrylic acid and salts thereof, carboxyvinyl polymers and salts thereof, alginic acid and salts thereof, propylene glycol alginate, chitosan, hydroxypropylcellulose, hydroxypropylmethylcellulose, hydroxyethylcellulose, ethylcellulose, methylcellulose, polyvinyl alcohol, polyvinyl pyrrolidone, N-vinylacetamide polymer, polyvinyl methacrylate, polyethylene glycol, pluronic, gelatin, methyl vinyl ether-maleic anhydride copolymer, soluble starch, pullulan and a copolymer of methyl acrylate and 2-ethylhexyl acrylate lecithin, lecithin derivatives, propylene glycol fatty acid esters, glycerin fatty acid esters, sorbitan fatty acid esters, polyoxyethylene sorbitan fatty acid esters, polyethylene glycol fatty acid esters, polyoxyethylene hydrated caster oil, polyoxyethylene alkyl ethers, and pluronic and appropriate buffer system in pH range of 6.5 to 8. The use of preservatives, masking agents is suitable. The average diameter of micronized particles can be between 1 and 20 micrometers, or can be less than 1 micrometer. Compounds can also be incorporated in the formulation by using their water-soluble salt forms.

Alternatively, materials may be incorporated into the matrix of the tablet e.g. hydroxypropyl methylcellulose, ethyl cellulose or polymers of acrylic and metacrylic acid esters. These latter materials may also be applied to tablets by compression coating.

Pharmaceutical compositions can be prepared by mixing a therapeutically effective amount of the active substance with a pharmaceutically acceptable carrier that can have different forms, depending on the way of administration. Pharmaceutical compositions can be prepared by using conventional pharmaceutical excipients and methods of preparation. The forms for oral administration can be capsules, powders or tablets where usual solid vehicles including lactose, starch, glucose, methylcellulose, magnesium stearate, di-calcium phosphate, mannitol may be added, as well as usual liquid oral excipients including, but not limited to, ethanol, glycerol, and water. All excipients may be mixed with disintegrating agents, solvents, granulating agents, moisturizers and binders. When a solid carrier is used for preparation of oral compositions (e.g., starch, sugar, kaolin, binders disintegrating agents) preparation can be in the form of powder, capsules containing granules or coated particles, tablets, hard gelatin capsules, or granules without limitation, and the amount of the solid carrier can vary (between 1 mg to 1 g). Tablets and capsules are the preferred oral composition forms.

Pharmaceutical compositions containing the compounds of the present invention may be in any form suitable for the intended method of administration, including, for example, a solution, a suspension, or an emulsion. Liquid carriers are typically used in preparing solutions, suspensions, and emulsions. Liquid carriers contemplated for use in the practice of the present invention include, for example, water, saline, pharmaceutically acceptable organic solvent(s), pharmaceutically acceptable oils or fats, and the like, as well as mixtures of two or more thereof. The liquid carrier may contain other suitable pharmaceutically acceptable additives such as solubilizers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols. Suitable oils include, for example, soybean oil, coconut oil, olive oil, safflower oil, cottonseed oil, and the like. For parenteral administration, the carrier can also be an oily ester such as ethyl oleate, isopropyl myristate, and the like. Compositions of the present invention may also be in the form of microparticles, microcapsules, liposomal encapsulates, and the like, as well as combinations of any two or more thereof.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for oral compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable fillers for oral compositions include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of suitable pharmaceutically acceptable flavourings for the oral compositions include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Suitable examples of pharmaceutically acceptable sweeteners for the oral compositions include, but are not limited to, aspartame, saccharin, saccharin sodium, sodium cyclamate, xylitol, mannitol, sorbitol, lactose and sucrose.

Suitable examples of pharmaceutically acceptable buffers include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants include, but are not limited to, sodium lauryl sulfate and polysorbates.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

Suitable examples of pharmaceutically acceptable stabilizers and antioxidants include, but are not limited to, ethylenediaminetetraacetic acid (EDTA), thiourea, tocopherol and butyl hydroxyanisole.

The compounds of the invention may also, for example, be formulated as suppositories e.g., containing conventional suppository bases for use in human or veterinary medicine or as pessaries e.g., containing conventional pessary bases.

The compounds according to the invention may be formulated for topical administration, for use in human and veterinary medicine, in the form of ointments, creams, gels, hydrogels, lotions, solutions, shampoos, powders (including spray or dusting powders), pessaries, tampons, sprays, dips, aerosols, drops (e.g., eye ear or nose drops) or pour-ons.

For application topically to the skin, the compound of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax, sorbitan monostearate, a polyethylene glycol, liquid paraffin, polysorbate 60, cetyl esters wax, cetearyl alcohol, 2-octyldodecanol, benzyl alcohol, and water. Such compositions may also contain other pharmaceutically acceptable excipients, such as polymers, oils, liquid carriers, surfactants, buffers, preservatives, stabilizers, antioxidants, moisturizers, emollients, colorants, and flavourings.

Examples of pharmaceutically acceptable polymers suitable for such topical compositions include, but are not limited to, acrylic polymers; cellulose derivatives, such as carboxymethylcellulose sodium, methylcellulose or hydroxypropylcellulose; natural polymers, such as alginates, tragacanth, pectin, xanthan and cytosan.

As indicated, the compound of the present invention can be administered intranasally or by inhalation and is conveniently delivered in the form of a dry powder inhaler or an aerosol spray presentation from a pressurized container, pump, spray or nebulizer with the use of a suitable propellant, e.g., a hydrofluoroalkane such as 1,1,1,2-tetrafluoroethane (HFA 134AT) or 1,1,1,2,3,3,3-heptafluoropropane (HFA 227EA), or a mixture thereof. In the case of a pressurized aerosol, the dosage unit may be determined by providing a valve to deliver a metered amount. The pressurized container, pump, spray or nebulizer may contain a solution or suspension of the active compound, e.g., using a mixture of ethanol and the propellant as the solvent, which may additionally contain a lubricant, e.g., sorbitan trioleate.

Capsules and cartridges (made, for example, from gelatin) for use in an inhaler or insufflator may be formulated to contain a powder mix of the compound and a suitable powder base such as lactose or starch.

For topical administration by inhalation the compound according to the invention may be delivered for use in human or veterinary medicine via a nebulizer.

If the compound of the present invention is administered parenterally, then examples of such administration include one or more of: intravenously, intraarterially, intraperitoneally, intrathecally, intraventricularly, intraurethrally, intrasternally, intracranially, intramuscularly or subcutaneously administering the agent, and/or by using infusion techniques.

For parenteral administration, the compound is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The compounds according to the invention may be formulated for use in human or veterinary medicine by injection (e.g. by intravenous bolus injection or infusion or via intramuscular, subcutaneous or intrathecal routes) and may be presented in unit dose form, in ampoules, or other unit-dose containers, or in multi-dose containers, if necessary with an added preservative. The compositions for injection may be in the form of suspensions, solutions, or emulsions, in oily or aqueous vehicles, and may contain formulatory agents such as suspending, stabilising, solubilising and/or dispersing agents. Alternatively the active ingredient may be in sterile powder form for reconstitution with a suitable vehicle, e.g. sterile, pyrogen-free water, before use.

The pharmaceutical compositions of the invention may contain from 0.01 to 99% weight per volume of the active material. For topical administration, for example, the composition will generally contain from 0.01-10%, more preferably 0.01-1% of the active compound.

A therapeutically effective amount of the compound of the present invention can be determined by methods known in the art. The therapeutically effective quantities will depend on the age and on the general physiological condition of the subject, the route of administration and the pharmaceutical formulation used. The therapeutic doses will generally be between about 10 and 2000 mg/day and suitably between about 30 and 1500 mg/day. Other ranges may be used, including, for example, 50-500 mg/day, 50-300 mg/day, 100-200 mg/day. The daily dose as employed for acute human treatment will range from 0.01 to 250 mg/kg body weight, suitably 2-100 mg/kg body weight, or suitably 5-60 mg/kg body weight, which may be administered in one to four daily doses, for example, depending on the route of administration and the condition of the subject. When the composition comprises dosage units, each unit will contain 10 mg to 2 g of active ingredient, suitably 200 mg to 1 g of active ingredient.

Administration may be once a day, twice a day, or more often, and may be decreased during a maintenance phase of the disease or disorder, e.g. once every second or third day instead of every day or twice a day. The dose and the administration frequency will depend on the clinical signs with the reduction or absence of at least one or more, preferably more than one, clinical signs of the acute phase known to the person skilled in the art. In one aspect of the present invention, administration is once daily oral dosing.

In one aspect, the present invention provides a combination comprising a) a compound of Formula (I) or a pharmaceutically acceptable salt thereof and b) one or more further therapeutically active agents.

The combinations referred to above may conveniently be presented for use in the form of a pharmaceutical composition and thus pharmaceutical compositions comprising a combination as defined above together with one or more pharmaceutically acceptable carriers thereof represent a further aspect of the invention.

The individual components of such combinations may be administered either sequentially or simultaneously in separate or combined pharmaceutical formulations. Appropriate doses of known therapeutic agents will be readily appreciated by those skilled in the art.

### Methods of Preparation:

Compounds of Formula (I) and salts thereof may be prepared by the general methods outlined hereinafter or any method known in the art, said methods constituting a further aspect of the invention. In the following description, the groups A, R¹, R², R³, R⁴, R⁵, R⁶, R⁷, R⁸, R⁹, R¹⁰, R¹¹, R¹², R¹³, R¹⁴, R¹⁵ and a have the meaning defined for the compounds of Formula (I) unless otherwise stated.

It will be appreciated by those skilled in the art that it may be desirable to use protected derivatives of intermediates used in the preparation of the compounds of Formula (I). Protection and deprotection of functional groups may be performed by methods known in the art. Hydroxyl or amino groups may be protected with any hydroxyl or amino protecting group (for example, as described in Green and Wuts. Protective Groups in Organic Synthesis. John Wiley and Sons, New York, 1999). The protecting groups may be removed by conventional techniques. For example, acyl groups (such as alkanoyl, alkoxycarbonyl and aryloyl groups) may be removed by solvolysis (e.g., by hydrolysis under acidic or basic conditions). Benzyl group may be cleaved by hydrogenolysis in the presence of a catalyst such as palladium-on-carbon. 1,2 diol groups may be protected as acetal by reaction with dimethyl acetal of N,N-dimethylacetamide (DMADMA) or dimethyl acetal of N,N-dimethylformamide (DMFDMA) which may be removed by hydrogenolysis or methanolysis at reflux (Tetrahedron Lett. 12 (1971), 813-816, Collection Czech. Chem. Commun. 32 (1967), 3159).

The synthesis of the target compound is completed by removing any protecting groups, which are present in the penultimate intermediate using standard techniques, which are well-known to those skilled in the art. The final product is then purified, as necessary, using standard techniques such as silica gel chromatography, HPLC on silica gel, and the like or by recrystallization.

Compounds of Formula (I) wherein R⁵ is C₁₋₄alkyl or C(O)C₁₋₃alkyl may be prepared by reaction of amine of Formula (II), with suitable acid of Formula (III) HOOCR⁶ in the presence of carbodiimides such as polymer-supported carbodiimide (PS CDI), 1-(3-dimethylaminopropyl)-3-ethylcarbodiimide (EDC), dicyclohexylcarbodiimide (DCC) or 1,8-diazabicyclo[5.4.0.]undec-7-ene (DBU) in the presence of hydroxybenzotriazole monohydrate (HOBt) in a suitable inert organic solvent such as a halohydrocarbon (e.g. dichloromethane), N,N-dimethylformamide or mixture thereof optionally in the presence of a tertiary organic base such as dimethylaminopyridine or triethylamine or in the presence of an inorganic base (eg. sodium hydroxide) and at a temperature in the range 0° to 120° C.

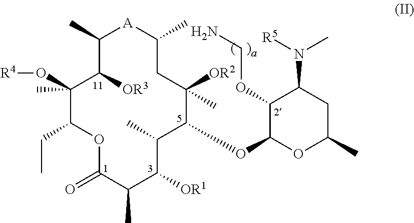

Compounds of Formula (I) wherein R⁵ is C₁₋₄alkyl may be prepared by alkylation of compounds of Formula (I) wherein R⁵ is H, for example where R⁵ is methyl by alkylating a chloroform solution of the compound wherein R⁵ is H with formaldehyde in the presence of formic acid. Compounds of Formula (I) wherein R⁵ is C₂₋₄alkyl may also be prepared by reductive N-alkylation of compounds of Formula (I) wherein R⁵ is H with aldehyde of Formula C₁₋₃alkylC(O)H.

Compounds of Formula (I) wherein R⁵ is —C(O)C₁₋₃alkyl may be prepared from compounds of Formula (I), wherein R⁵ is H, R³, R⁴ are suitable hydroxy protecting groups and R¹ represents α-L-cladinosyl group of formula (a) having C/4″-hydroxyl protecting group, by reaction with the appropriate carboxylic acid C₁₋₃alkylC(O)OH in the presence of DCC and DMAP in a suitable solvent such as dichloromethane.

Compounds of Formula (I) wherein R⁵ is hydrogen may be prepared by reaction of a compound of Formula (I), wherein R⁵ is C₁₋₄alkyl, suitably methyl, by conventional techniques for mono-demethylation of the 3′-NMe₂ group, for example by reaction with iodine under UV radiation (preferably with 500 W halogen lamp), in the presence of sodium acetate trihydrate (U.S. Pat. No. 3,725,385 and WO2004/013153), or by reaction of compound of Formula (I) with N-iodosuccinimide in acetonitrile at room temperature (J. Org. Chem. 65 (2000) 3875-3876), or with iodine in presence of morpholine or with benzylchloroformate, followed by elimination of benzyloxycarbonyl groups at position 2′ and 3′ as described in U.S. Pat. No. 5,250,518.

Compounds of Formula (II) wherein a is an integer from 2 to 6, and R⁵ is C₁₋₄alkyl or C(O)C₁₋₃alkyl may be prepared from compounds of Formula (IV) wherein a′ is an integer from 1 to 5

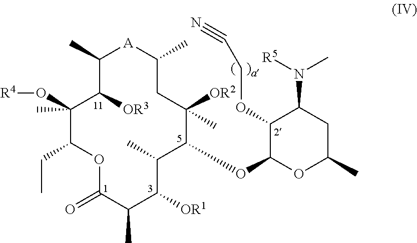

by reduction of the cyano nitrogen to —NH₂.

The reaction is suitably carried out in a suitable solvent such as acetic acid using suitable reduction conditions, such as hydrogenation in the presence of a suitable catalyst such as platinum dioxide at a suitable pressure, such as a pressure in the range 3 to 7 barr, suitably 5 barr.

Compounds of Formula (IV) wherein a′ is 2 may be prepared by reaction of a compound of Formula (V) wherein R³, R⁴ are suitable hydroxy protecting groups and R¹ represents an α-L-cladinosyl group of formula (a) having a C/4″-hydroxyl protecting group

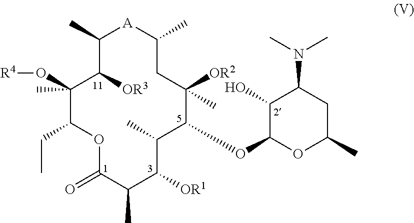

with acrylonitrile, in the presence of a strong base, such as NaOH, KOtBu, NaOtBu or NaH, in a suitable solvent such as DMSO or t-BuOH.

Compounds of Formula (IV) wherein a′ is 1 may be prepared by reaction of a compound of Formula (V) wherein R³, R⁴ are suitable hydroxy protecting groups and R¹ represents an α-L-cladinosyl group of formula (a) having a C/4″-hydroxyl protecting group, by reaction with a suitable monohalogenated acetonitrile, for example chloracetonitrile, in the presence of a strong base, such as NaOH, KOtBu, NaOtBu or NaH, in a suitable solvent such as DMSO or t-BuOH.

Compounds of Formula (IV) wherein a′ is an integer from 3 to 5 may be prepared by reaction of a compound of Formula (VI) wherein R³, R⁴ are suitable hydroxy protecting groups and R¹ is represents an α-L-cladinosyl group of formula (a) having a C/4″-hydroxyl protecting group,

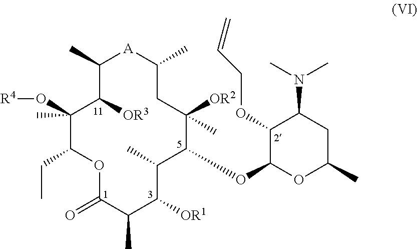

by reaction with a compound of Formula (VII)

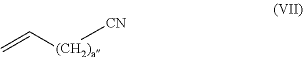

wherein a″ is an integer from 0 to 2, under conditions of Grubbs metathesis (A. K. Chatterjee, T.-L. Choi, D. P. Sanders, R. H. Grubbs, JACS 125 (2003) 11360). Selective reduction of the double bond (and not the —CN group) may be achieved by hydrogenation in the presence of Pd/C catalyst in a suitable solvent, such as an alcohol such as ethanol or methanol (J. Med. Chem 51 (2008) 424-431).

Compounds of Formula (II) wherein a is an integer of 5 or 6, and R⁵ is C₁₋₄alkyl or C(O)C₁₋₃alkyl may also be prepared from compounds of Formula (VI) and (VII) using Grubbs metathesis as described above, but using acidic reduction conditions such as hydrogenation in the presence of a suitable catalyst such as platinum dioxide at a suitable pressure, such as a pressure in the range 3 to 7 barr, suitably 5 barr, in a suitable solvent such as acetic acid.

Compounds of Formula (VI) may be prepared by palladium-catalysed allylation of compounds of Formula (V) wherein R³, R⁴ are suitable hydroxy protecting groups and R¹ is represents an α-L-cladinosyl group of formula (a) having a C/4″-hydroxyl protecting group, for example according to the procedure described in WO 2006/120541 for Intermediate 16.

Compounds of Formula (V) and compounds of formula (I) wherein R³ and R⁴ taken together with the intervening atoms form a cyclic carbonate group of Formula (b);

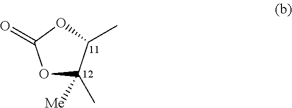

may be prepared by analogous methods to those known in the art from compounds of Formula (V) and from compounds of formula (I), respectively wherein R³ and R⁴ are H. Thus, they can be prepared according to the procedure in J. Antibiot. 40 (1987), 1006-1015 and EP0307177.

Compounds of Formula (V) wherein A represents —C(O)— and R² is methyl, may be prepared according to J. Antibiotics 37 (1984) 187-189.

Compounds of Formula (V) wherein A represents —C(═NOR⁹)— and R² is hydrogen or methyl, may be prepared according to U.S. Pat. No. 3,478,014 or J. Antibiotics 44 (1991) 313-330.

Compounds of Formula (V) wherein A represents —CH(OH)— may be prepared from compounds of formula (V) where A is —C(O)— using reducing agents, for instance hydrides (sodium borohydride lithium borohydride, sodium cyano borohydride or lithium aluminium hydride) according to J. Antibiotics (1990) 1334-1336.

Compounds of Formula (V) wherein A represents —N(R⁹)CH₂— or —CH₂N(R⁹)— may be obtained by reduction of the corresponding 9a- or 8a-imino ether followed by reductive N-alkylation according to the procedure described in J. Chem. Soc. Perkin Trans (1986) 1881-1890, J. Chem Res. S (1988) 152-153; (M) (1988) 1239-1261, J. Antibiotics 41 (1988) 1029-1047 and EP0508725.

Compounds of Formula (V) wherein A represents —CH(NR¹⁰R¹¹)—, and R¹⁰ and R¹¹ are H may be prepared as described in Tetrahedron Lett., 11, (1970) 157-160.

Compounds of Formula (V) wherein A represents —CH(NR¹⁰R¹¹)—, and R¹⁰ and R¹¹ are —C₁₋₆alkyl or —C(O)R⁹ may be prepared according to the procedure described in J. Med. Chem., 16 (1973), 1059-1060.

Compounds of Formula (V) wherein A represents —CH(NR¹⁰R¹¹)—, and R¹⁰ and R¹¹ are —C₁₋₆alkyl may also be prepared according to the procedure described in J. Antibiotics 41 (1988) 1029-1047.

Compounds of Formulae III and VII are commercially available or may be readily prepared by methods well known in the art.

Pharmaceutically acceptable acid addition salts, which also represent an object of the present invention, may be obtained by reaction of a compound of Formula (I) with an at least equimolar amount of the corresponding inorganic or organic acid such as hydrochloric acid, hydroiodic acid, sulfuric acid, phosphoric acid, acetic acid, trifluoroacetic acid, propionic acid, benzoic acid, benzenesulfonic acid, methane sulfonic acid, laurylsulfonic acid, stearic acid, palmitic acid, succinic acid, ethylsuccinic acid, lactobionic acid, oxalic acid, salicylic acid and similar acid, in a solvent inert to the reaction. Addition salts are isolated by evaporating the solvent or, alternatively, by filtration after a spontaneous precipitation or a precipitation by the addition of a non-polar cosolvent.

### Biological Assays

The potential for a compound of the present invention to have an advantageous profile for providing therapeutic benefit in the treatment of neutrophil dominated inflammatory diseases resulting from neutrophilic infiltration and/or diseases associated with altered cellular functionality of neutrophils may be demonstrated, for example, using the following assays.

A compound analysed using biological assays defined herein is considered to be active if it exhibits at least one of the following results:
a) 40% or more, suitably 50% or more, inhibition in the in vitro Inhibition of IL-6 production in LPS-stimulated murine spleenocytes assay; and/or
b) 40% or more, suitably 50% or more, inhibition in the in vivo Lung neutrophilia induced by bacterial lipopolysaccharide in male BALB/cJ mice—Method A; and/or
c) 30% or more, suitably 50% or more, inhibition in the in vivo Lung neutrophilia induced by bacterial lipopolysaccharide in male BALB/cJ mice—Method B; and/or
d) it statistically significantly reduced neutrophil numbers in BALF in the in vivo Cigarette-smoke-induced lung neutrophilia assay.

The following abbreviations are used in the text: cfu for colony forming unit, DMSO for dimethyl sulfoxide, LPS for bacterial lipopolysaccharide, PBS for phosphate buffered saline and BAL for bronchoalveolar lavage.

### In Vitro Screening Protocol

### Compound Preparation

Test and reference substances used in an in vitro assays were dissolved in dimethyl sulfoxide (DMSO) (Sigma Chemical Co., USA) at a concentration of 50 mM and were further diluted to test concentrations in appropriate cell culture medium.

### Inhibition of IL-6 Production in LPS-Stimulated Murine Spleenocytes In Vitro

After cervical dislocation, mouse spleens were removed using sterile dissection tools. Spleens were transferred to a pre-wetted cell strainer in a 50 mL sterile conical tube and cell suspension was made by gentle puddle. Cells were centrifuged (20 min, 300×g) and resuspended in 2 mL of sterile phosphate buffered saline (PBS) (Sigma Chemical Co., USA). Red blood cells were lysed by addition of 3 mL of sterile water and occasionally gentle shaking for 1 minute. Afterwards, the tube was filled to 40 mL with DMEM medium and centrifuged (20 min, 300×g). Cells were resuspended in DMEM supplemented with 1% FBS and seeded in a 24-well plate, 1×10⁶ cells per mL medium.

Final concentrations of 50 μM, 25 μM, 12.5 μM, 6.3 μM and 3.1 μM of test compounds were prepared by diluting a 50 mM DMSO stock in Dulbecco's modified Eagle medium (DMEM) (Gibco, USA) supplemented with 1% heat inactivated fetal bovine serum (FBS) (BioWest, Ringmer, United Kingdom).

Cells were pre-incubated with the test compounds for 3 h at 37° C., in an atmosphere of 5% CO₂ and 90% humidity. Afterwards, cells were stimulated with 1 μg/mL lipopolysaccharide (LPS, E. coli 0111:B4, Sigma Chemical Co., USA) and incubated overnight. Concentration of IL-6 was determined in cell supernatants by sandwich ELISA using capture and detection antibodies (R&D Systems, USA) according to the manufacturer's recommendations.

Inhibition (as percentage) was calculated using the following formula:

% inhibition=[1−(concentration of IL-6 in sample−concentration of IL-6 in negative control)/(concentration of IL-6 in positive control−concentration of IL-6 in negative control)]×100.

The positive control refers to LPS-stimulated samples that were not preincubated with the compounds.

The negative control refers to unstimulated and untreated samples.

### Cytotoxicity Assay in THP-1 and HepG2 Cell Lines

THP-1 cells were grown in RPMI 1640 medium (Institute of Immunology, Zagreb) supplemented with 10% fetal bovine serum (FBS; BioWest), 50 U/ml penicillin, 50 μg/ml streptomycin, and 2.5 μg/ml amphotericin B (Fungizone) (all from Gibco).

HepG2 cells were maintained in Eagle's minimal essential medium (MEM; Gibco) containing 10% FBS, 1 mM sodium pyruvate, 0.1 mM nonessential amino acids, 50 U/ml penicillin, 50 μg/ml streptomycin, and 2.5 μg/ml amphotericin B (Fungizone) (all from Gibco).

To determine whether the anti-inflammatory activity of the test compounds is due to observed inhibition of cytokine production and is not a consequence of cellular cytotoxicity, measurement of succinate dehydrogenase activity in living cells was performed. Cells were cultured for 24 h in appropriate tissue culture medium in the presence of test compounds at concentrations of 100, 50, 25, 12.5, 6.25, 3.13, 1.56, 0.78 μM. Final concentrations of 50 μM, 25 μM, 12.5 μM, 6.3 μM and 3.1 μM of test compounds were prepared by diluting a 50 mM DMSO stock in cell culture medium. Cell viability was determined by CellTiter 96® AQueous Assay (Promega, USA). The amount of MTS-formazan [[3-(4,5-dimethylthiazol-2-yl)-5-(3-carboxymethoxyphenyl)-2-(4-sulfophenyl)-2H-tetrazolium] produced was determined using a spectrophotometer at 492 nm (Mosmann, J. Immunol. Methods, (1983) 65: 55-63).

The percentage of inhibition of cell growth was calculated using the following formula:

% inhibition of cell growth=OD₄₉₂ treated cells/OD₄₉₂ nontreated cells×100.

### In Vivo Screening Protocols

### Lung Neutrophilia Induced by Bacterial Lipopolysaccharide (LPS) in Male BALB/cJ Mice

### Method A

For intraperitoneal administration (i.p.) compounds were dissolved in a final concentration of 10 mg/mL. The required amount of compound was first dissolved in dimethylsulfoxide (DMSO, Sigma) and then diluted with 0.5% (w/v) methyl-cellulose so that the final DMSO concentration was 5% (v/v). The obtained solution was applied in a dose volume of 0.2 mL per 10 g of animal. Therefore, the compound dose was 200 mg/kg.

Male BALB/cJ mice (Charles River, France), with an average weight of ˜30 g were randomly grouped (n=8 in testing group, 10 in positive control and 8 in negative control). Mice received intraperitoneally (i.p.) a single dose of 200 mg/kg of test compound. Two hours after administration, 2 μg of LPS (from Escherichia coli serotype 0111:B4, Sigma), dissolved in sterile PBS in a volume of 60 μL, was intranasally administered to all experimental groups except the negative control group, which received the same volume of vehicle (PBS). Animals were sacrificed approximately 24 hours after application of LPS in order to obtain bronchoalveolar lavage fluid (BALF), which was used to determine absolute number of cells and the percentage of neutrophils. Results are expressed as percentage decrease in total cell number and number of neutrophils in BALF of treated animals compared to positive control (LPS challenged, but untreated animals).

### Method B

For intraperitoneal administration (i.p.) compounds were dissolved in a final concentration of 10 mg/mL. The required amount of compound was first dissolved in dimethylsulfoxide (DMSO, Sigma) and then diluted with 0.5% (w/v) methyl-cellulose so that the final DMSO concentration was 5% (v/v). The obtained solution was applied in a dose volume of 0.1 mL per 10 g of animal. Therefore, the compound dose was 100 mg/kg.

Male BALB/cJ mice (Charles River, Germany), with an average weight of ˜25 g were randomly grouped (n=8 in testing group, 10 in positive control and 6-8 in negative control). Mice received intraperitoneally (i.p.) a single dose of 100 mg/kg of test compound. Two hours after administration, 0.4 μg of LPS (from Escherichia coli serotype 0111:B4, Sigma), dissolved in sterile saline (0.9% NaCl) in a volume of 50 μL, was intranasally administered to all experimental groups except the negative control group, which received the same volume of vehicle (saline). Animals were sacrificed approximately 24 hours after application of LPS in order to obtain bronchoalveolar lavage fluid (BALF), which was used to determine absolute number of cells and the percentage of neutrophils. Results were expressed as percentage decrease in total cell number and number of neutrophils in BALF of treated animals compared to positive control (LPS challenged, but untreated animals), as revealed by cytospine evaluation. In some experiments neutrophil levels in BALF were assessed by measurement of concentration of a neutrophil-specific enzyme myeloperoxidase (MPO). Results are then expressed as percentage of decrease in myeloperoxidase (MPO) concentration measured in BALF lysates of treated animals compared to positive control (Mouse MPO ELISA Kit, Hycult biotechnology, Nederlands). For this purpose, BALF was sonicated after addition of 1.5% Triton-X-100 (Pharmacia Biotech) in Milli-Q water and frozen at −80° C. until analysed.

### Phorbol 12-Myristate 13-Acetate Induced Ear Edema in CD1 Mice

Male CD1 mice (Charles River, France) weighing 30-40 g were randomly grouped (n=8 in test group of which the untreated ear served as negative control; 8 in positive control group which also served as their own negative control group). Test compounds, as well as vehicle (Trans-phase Delivery system, containing benzyl alcohol 10%, acetone 40% and isopropanol 50%) (all from Kemika, Croatia), were administered topically to the internal surface of the left ear 30 minutes prior to administration of phorbol 12-myristate 13-acetate (PMA) (Sigma, USA). Test compounds were administered at a dose 500 μg in 15 μL per ear. 30 minutes later, 0.01% PMA solution in acetone was applied topically to the inner surface of the left ear of each animal in a volume of 12 μL per ear. During the treatment and challenge, animals were anaesthetized with anesthesia by inhalation. 6 h following challenge, animals were euthanized by intraperitoneal thiopental injection (PLIVA, Croatia). For assessing the auricular edema, 8 mm discs were cut out of left and right auricular pinna and weighed. The degree of edema was calculated by subtracting the weight of 8 mm disc of the untreated ear from that of the treated contralateral ear. The inhibition of edema is the treated animals is expressed as percentage compared to control mice.

### Cigarette-Smoke-Induced Lung Neutrophilia

Male BALB/c mice (weighing 21-24 g, were randomly grouped, 5 animals per group) were pre-treated with compound (100, 30, 10, 3 and 1 mg/kg body weight) or vehicle (5% DMSO/95% methyl-cellulose 0.5% w/v, positive control) by oral gavage in a volume of 10 mL/kg. 2 hours after compound administration, 10 mice were placed into a plexiglass box (Braintree Scientific, size 10″×4″×4″) with an input port for aerosol and another port for exhaust. Cigarette smoke was introduced through the aerosol port via a peristaltic pump (Masterflex L/S, Digital Economy Drive) set at 40 ml/min. Breathing air was also introduced through the same port via an elbow in the tubing at a rate of 0.4 L/min. With these settings, smoke concentration delivered to the box was approximately 10%. 3 cigarettes (research type 4A1, University of Kentucky Tobacco Institute) were given back-to-back with a 1 min period between each one, when the mice breathed fresh air for one minute. 2 to 3 hours after the first group of cigarettes, 2 additional cigarettes were given, again allowing 1 min between each cigarette. After the last cigarette, the mice were returned to their normal housing. This procedure was carried out for 2 days.

On the 3^{rd} day, mice were euthanized with 0.1 ml Fatal Plus i.p., then bronchoalveolar lavaged with 5×0.7 ml Dulbecco's phosphate-buffered saline (dPBS, in-house Media Prep lab) through a tracheal tube. BALF was spun at 3000 rpm for 10 min, the supernatant discarded, and the cells resuspended with 1 ml dPBS. Slides were made on a cytospin (100 μl fluid, spun at 300 rpm for 5 min), then stained with Diff Quick for differential counts (minimum of 200 cells counted). Total cell counts were performed with a hemocytometer after a dilution with Tuerks solution.

Results are expressed as percentage decrease in number of neutrophils in BALF of treated animals compared to positive control (vehicle treated and smoke challenged animals).

A compound is considered to be active if it statistically significantly reduced neutrophil numbers in BALF (p<0.05 vs vehicle control, 1-way ANOVA with Bonferroni post test, GraphPad Prism version 5.00 for Windows, GraphPad Software, San Diego Calif. USA, www.graphpad.com).

### Examples

The following abbreviations are used in the text: DMF-DMA for N,N-dimethylformamide-dimethylacetal, DMA/DMA for dimethylacetal-dimethylacetal, DMF for N,N-dimethylformamide, DCM for dichloromethane, DMAP for dimethylaminopyridine, EtOAc for ethyl acetate, DEA or Et₂N for diethylamine, TEA or Et₃N for triethylamine, IPA for isopropylamine, MeOH for methanol, t-BuOH for tert-butanol, BuOH for butanol, Et₂O for diethylether, HOBt for hydroxybenzotriazole monohydrate, HOAc for acetic acid, PS-Carbodiimide or PS-CDI for polymer-supported carbodiimide, DMSO for dimethyl sulfoxide, THF for tetrahydrofurane, and EDC or EDC×HCl for 1-ethyl-3(3-dimethylaminopropyl) carbodiimide hydrochloride, r.t. for room temperature, eq. for equivalents.

The compounds and processes of the present invention will be better understood in connection with the following examples, which are intended as an illustration only and not limiting the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Where reactions are described as having been carried out in a similar manner to earlier, more completely described reactions, the general reaction conditions used were essentially the same. Work up conditions used were of the types standard in the art, but may have been adapted from one reaction to another. In the procedures that follow, reference to the product of an Intermediate or Example by number is typically provided. This is provided merely for assistance to the skilled chemist to identify the starting material used. The starting material may not necessarily have been prepared from the batch referred to.

9-Deoxo-9a-methyl-9a-aza-9a-homoerythromycin A, may be prepared by the procedure as described in J. Chem. Res. (S) 1988, page 152.

### INTERMEDIATES

### Intermediate 1

### 2′-O-(3-Aminopropyl)-9-deoxo-9a-methyl-9a-aza-9a-homoerythromycin A

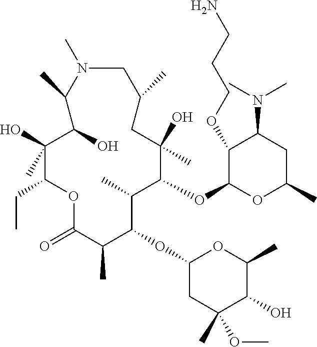

### Procedure A (WO2009016142)

### Step 1: Preparation of 3

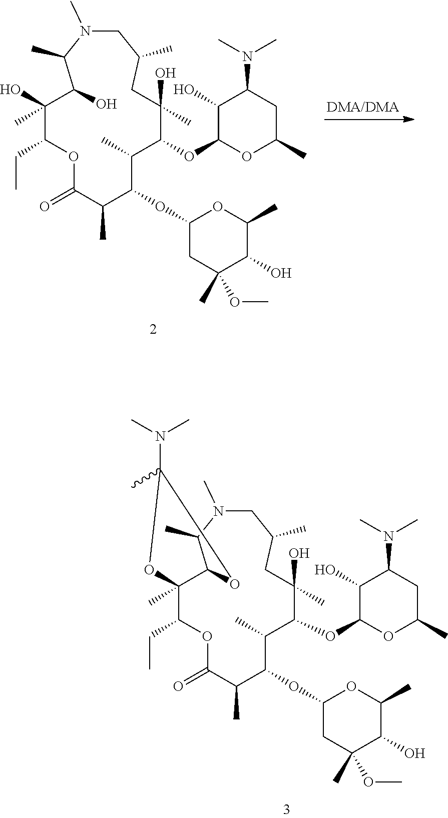

9-Deoxo-9a-methyl-9a-aza-9a-homoerythromycin A (2) (50 g, 66.7 mmol) was dissolved in chloroform (250 mL). The DMA/DMA (40 mL, 0.35 mol, 5.2 eq.) was added in one portion, and then heated at reflux temperature for 24 hours. The solvent was evaporated affording 45.28 g of the title product.

MS (ES+) m/z: 818 [MH]+.

### Step 2: Preparation of 4

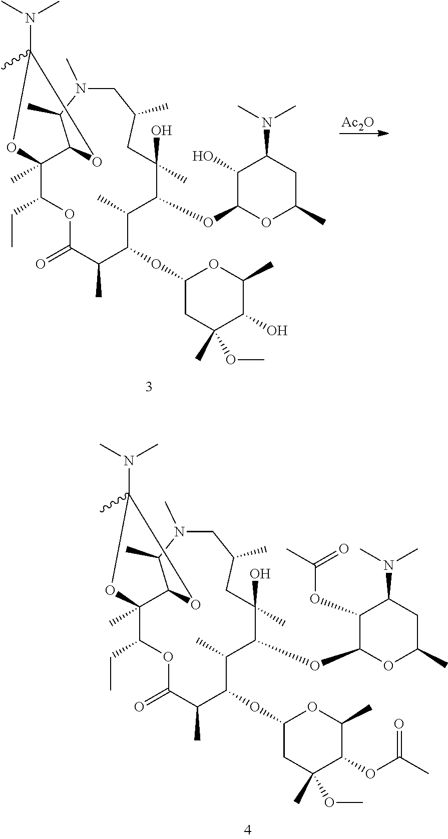

Compound 3 from step 1 (3 g, 3.66 mmol) was dissolved in DCM (70 mL) and cooled in the ice bath. In the reaction mixture Et₃N (3.2 mL, 6.2 eq.), DMAP (44.7 mg, 0.1 eq.) and acetone (1.9 mL, 5.4 eq.) were added. Temperature was allowed to slowly reach r.t. and the reaction mixture was stirred at r.t. for 28 hours. Reaction mixture was washed with saturated NaHCO₃ solution (100 mL) (pH 9). DCM layers were dried over Na₂SO₄ and concentrated under vacuum affording 2.78 g of the title product. Crude product was recrystalised from Et₂O and further from acetonitrile/H₂O yielding 2.13 g of the title product as a white powder.

MS (ES+) m/z: 902.17 [MH]+

### Step 3: Preparation of 5a and 5b

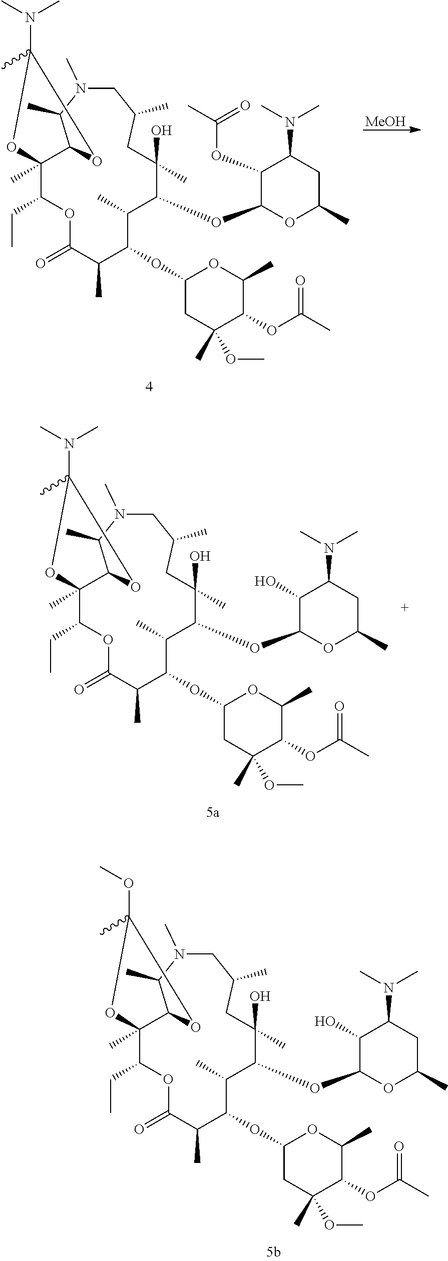

Compound 4 from step 2 (2.13 g, 2.36 mmol) was dissolved in MeOH (50 mL) and stirred at r.t. for 21 hours. Methanol was evaporated under vacuum to afford 2.08 g of the title product as white solid.

MS (ES+) m/z: 860 [MH]+ 5a
847 [MH]+ 5b

### Step 4: Preparation of 6a and 6b

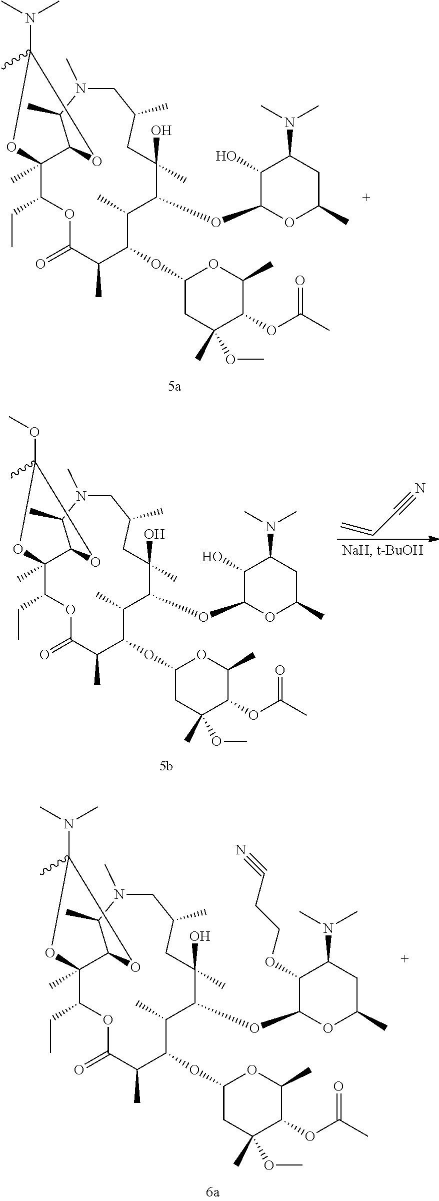

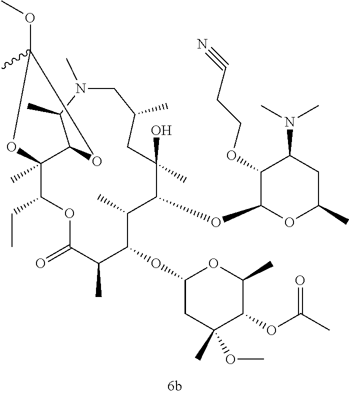

To a stirred mixture of 5a and 5b from step 3 (1.94 g, 2.25 mmol) in acrylonitrile (12 mL), t-BuOH (0.94 mL, 4.4 eq) was added at r.t. under nitrogen and the reaction mixture was cooled to 0° C. NaH (60% in mineral oil, 60 mg, 2.47 mmol, 1.1 eq.) was added in small amounts during 15 minutes. Temperature was allowed to slowly reach r.t. After 24 hours of stirring acrylonitrile was evaporated under reduced pressure. The polymer of acrylonitrile was precipitated in EtOAc/n-hexane and filtered off. The mother liquor was evaporated to afford oily product, which was then dissolved in EtOAc and extracted with water. EtOAc layers were collected and dried over Na₂CO₃. The solvent was evaporated under vacuum affording 1 g of title product.

MS (ES+) m/z: 914.10 [MH]+ 6a

MS (ES+) m/z: 900.15 [MH]+ 6b

### Step 5: Preparation of 7

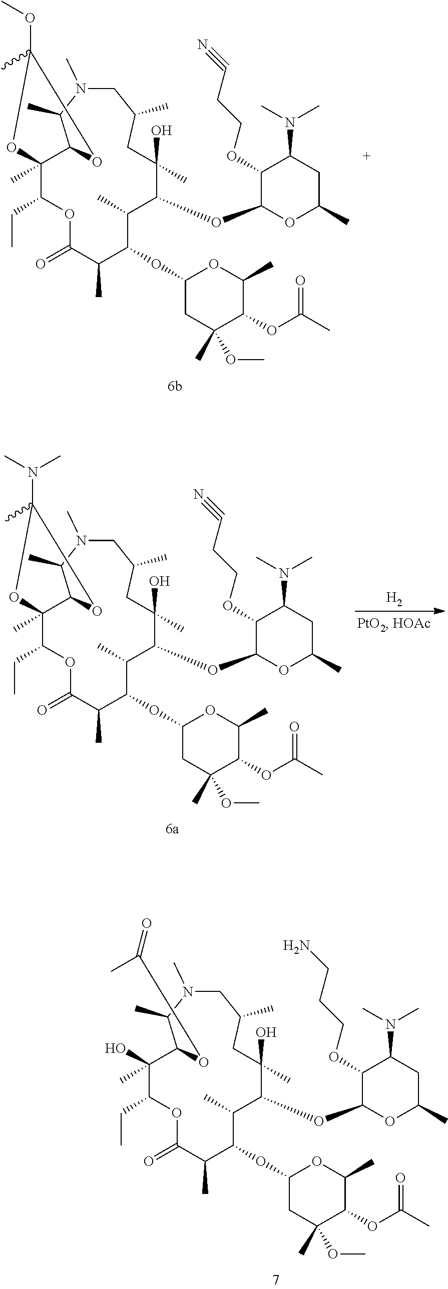

Compound 6a and 6b from step 4 (1 g), used without purification, was dissolved in glacial HOAc (10 mL) and hydrogenated with PtO₂ (100 mg) at 5 barr of H₂-pressure for 24 hours at r.t. The catalyst was filtered off and mother liquor evaporated under reduced pressure. The residue was dissolved in water and DCM, pH adjusted to 9, and extracted with DCM (150 mL). DCM layers were collected and dried over Na₂SO₄. The solvent was evaporated under reduced pressure affording 560 mg of white powder. Crude product was recrystalised from EtOAc/n-hexane yielding 486.4 mg of the title product.

MS (ES+) m/z: 890.5 [MH]+

### Step 6: Preparation of Intermediate 1

### 2′-O-(3-Aminopropyl)-9-deoxo-9a-methyl-9a-aza-9a-homoerythromycin A

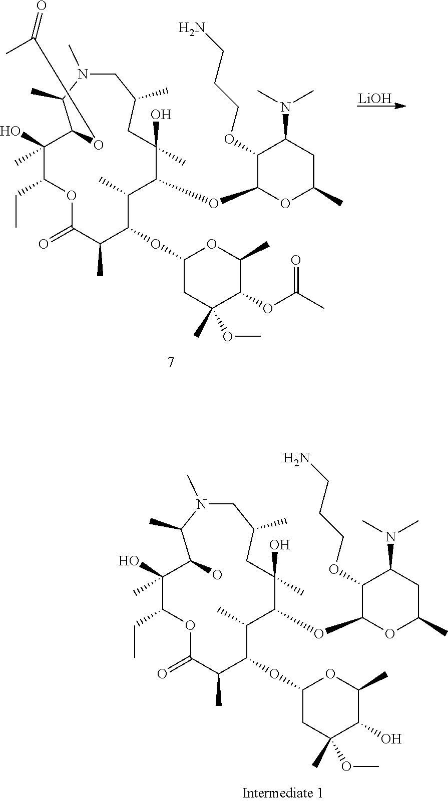

Compound 7 from step 5 (486 mg, 0.54 mmol) was dissolved in THF (4.5 mL), LiOH (6 mL of 0.5 M) was added, heated at 40° C. for 2 hours and stirred at r.t. for 72 hours. H₂O (10 mL) was added to the reaction mixture, followed by extraction with EtOAc. Organic layers were collected and dried over Na₂SO₄. Solvent was evaporated yielding 380 mg of the title product as a white solid.

MS (ES+): 806 [MH]+

¹³C-NMR (CDCl₃) δ/ppm: 178.9, 102.6, 94.4, 82.4, 80.6, 78.3, 77.3, 77.1, 74.1, 73.7, 73.4, 72.9, 72.6, 70.0, 68.3, 65.4, 64.4, 62.9, 49.8, 45.6, 42.5, 39.7, 39.6, 36.3, 34.6, 29.0, 27.4, 26.6, 26.5, 22.3, 21.6, 21.5, 18.4, 16.4, 14.5, 11.3, 8.5, 7.6.

### Intermediate 1

### Procedure B1 (WO2009016142)

### Step 1: Preparation of 8

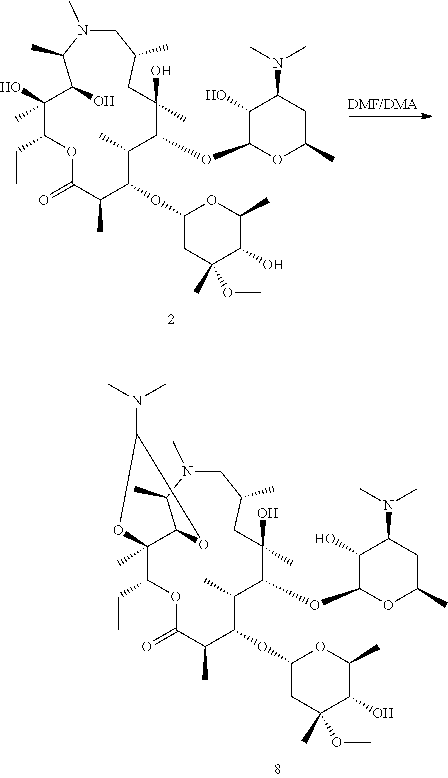

To a solution of 9-deoxo-9a-methyl-9a-aza-9a-homoerythromycin A (2) (20 g, 0.027 mol) in CHCl₃ (75 mL) DMF/DMA was added (12.5 mL, 0.093 mol), reaction was stirred at 65° C. for 5 hours and then placed at r.t. for the next 17 hours. After that time, additional DMF/DMA (12.5 mL) was added and the reaction mixture was stirred for a further 5 hours at r.t. After completion of the reaction, solvent was evaporated yielding 26.58 g of the title product as light yellow amorphous solid.

MS (ES+): 804.6 [MH]⁺

### Step 2: Preparation of 9

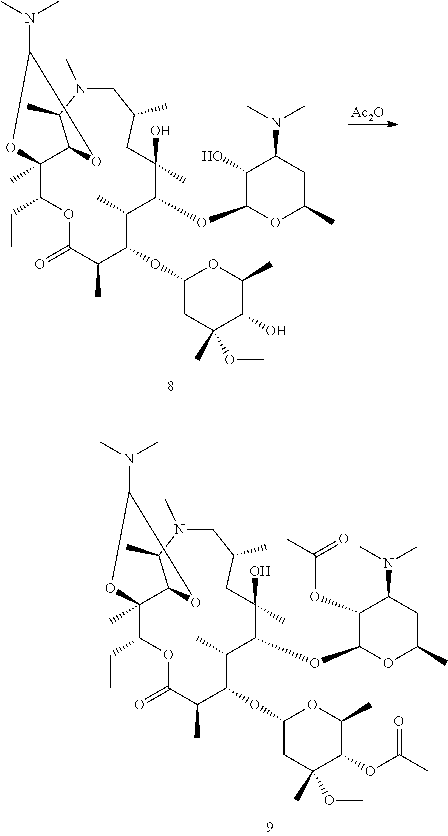

To a solution of compound 8 from step 1 (26.5 g, 0.033 mol, used without purification) in DCM (250 mL), triethylamine (28.6 mL, 0.2 mol) and DMAP (0.403 g, 0.0033 mol) were added. Solution was then cooled at 0° C. and acetic acid anhydride was added dropwise (17.1 mL, 0.18 mol). The reaction mixture stirred at r.t. for 4 hours. The organic layer was washed twice with saturated solution of NaHCO₃ then with water and brine. After drying over Na₂SO₄ and evaporation of solvent, crude product was recrystalised from diethylether to afford 8.3 g of the title product as a white crystals. After evaporating of mother liquor an additional 20.1 g of the title product was isolated. (MS (ES⁺): 888.6 [MH]⁺;

### Step 3: Preparation of 10b

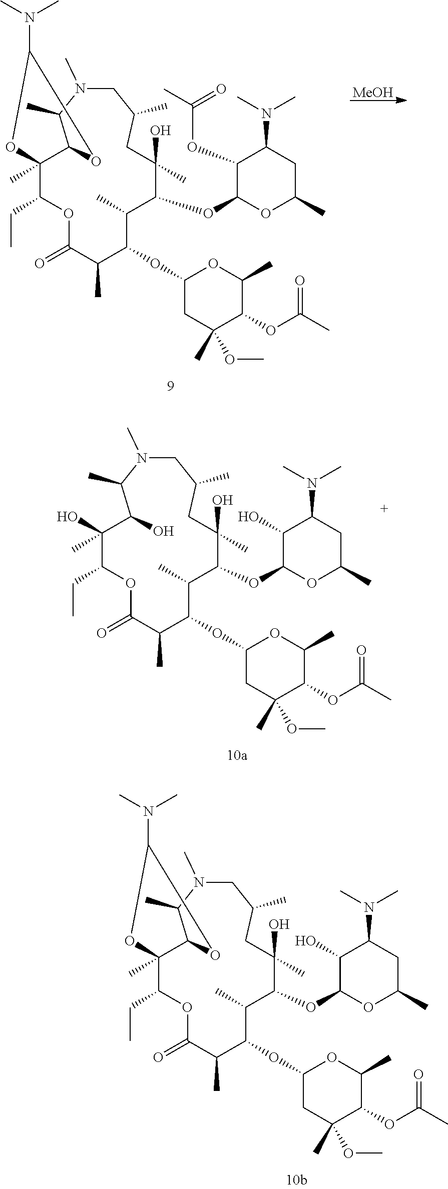

Compound 9 from step 2 (3.1 g, 3.49 mmol) was dissolved in MeOH (125 mL) and stirred at r.t. for 72 hours. Solvent was evaporated affording 2.55 g of the mixture 10a and 10b as a brown powder.

MS (ES+): 791 [MH]⁺; 10a
846 [MH]⁺ 10b

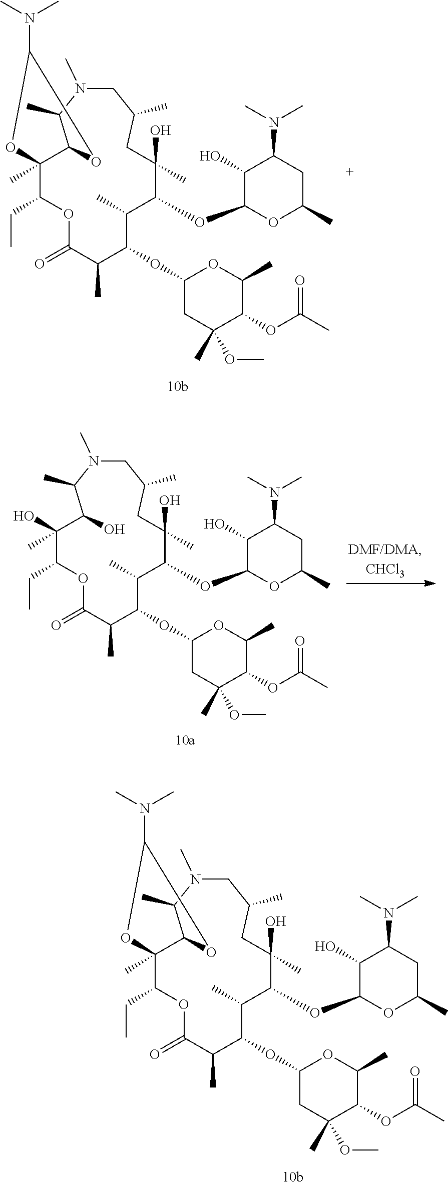

To the mixture of unprotected 10a and protected compound 10b (2.49 g) in CHCl₃ (15 mL) DMF/DMA (3 mL) was added and reaction mixture was stirred at 65° C. for 3 hours. Reaction mixture was cooled at r.t., solvent evaporated affording 3 g of the title product as a light yellow solid.

MS (ES+): 846 [MH]⁺

### Step 4: Preparation of 11a and 11b

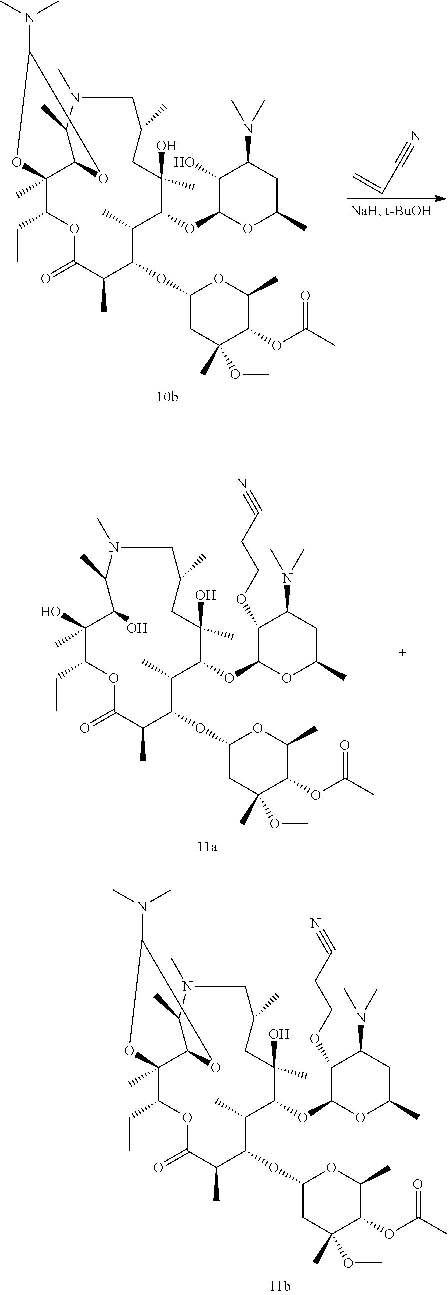

Compound 10b from step 3 (2.56 g, 3.03 mmol) was dissolved in acrylonitrile (25 mL), t-BuOH (1.5 mL, 15.9 mmol) was added and the reaction mixture was cooled at 0° C., followed by addition of NaH (112 mg, 3.33 mmol, 60% suspension in mineral oil). Reaction mixture was stirred for 3 hours and then evaporated. The residue was dissolved in EtOAc and washed with water and brine. Organic layers were dried over Na₂SO₄ and solvent was evaporated yielding 2.4 g of brown oil product which was further purified by column chromatography (DCM:MeOH:NH₄OH 90:9:0.5) yielding 1.69 g of title products.

MS (ES+): 844 [MH]⁺ 11a
899 [MH]⁺ 11b

### Step 5: Preparation of 12

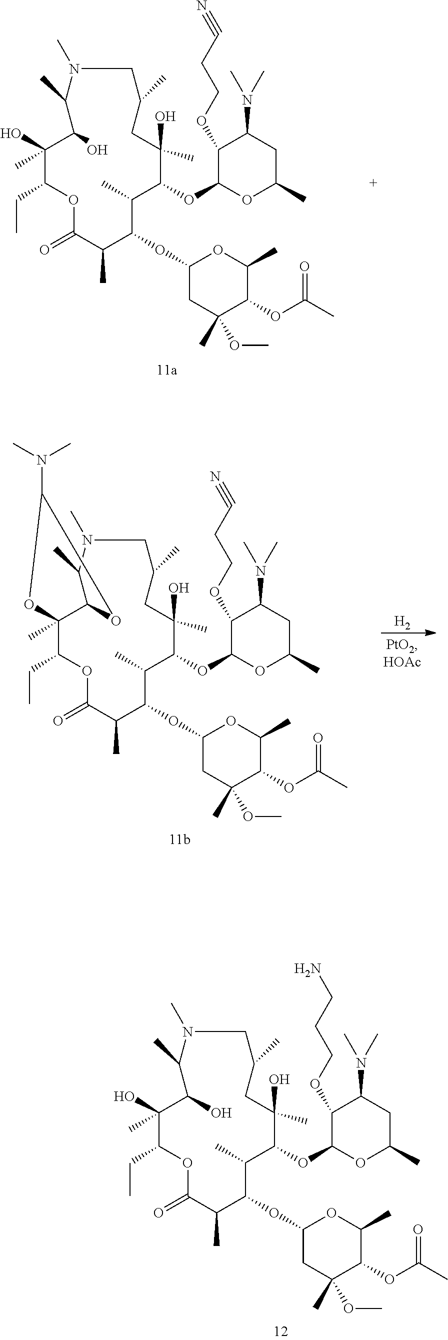

To a solution of 11a and 11b from step 4 (1.69 g) in HOAc (30 mL), PtO₂ (301 mg) was added and the reaction mixture was stirred at r.t. under 5 barr of H₂-pressure for 20 hours. The catalyst was filtered off, solvent was evporated and residue dissolved in water and DCM. pH value was adjusted to 9.3 by addition of 1M NaOH and product was extracted with DCM. Collected organic layers were dried over Na₂SO₄ and solvent was evaporated. Crude product was recrystalised from EtOAc/n-hexane yielding 1.23 g of the title product as a white powder.

MS (ES+): 848.5 [MH]⁺

### Step 6: Preparation of Intermediate 1

### 2′-O-(3-Aminopropyl)-9-deoxo-9a-methyl-9a-aza-9a-homoerythromycin A

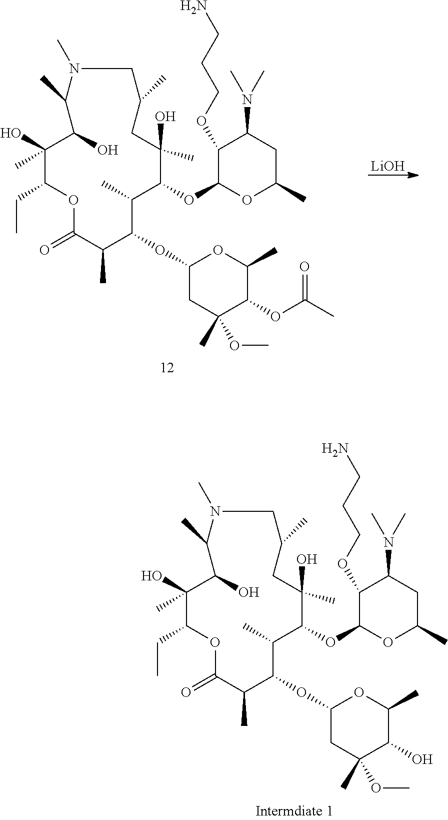

To a solution of 12 from step 5 (1.67 g, 1.97 mmol) in THF (15 mL), LiOH (10 mL of 0.5M solution in H₂O) was added. The reaction mixture was stirred for 5 hours at 65° C. and additionaly 72 hours at r.t. The reaction mixture was diluted with EtOAc and washed with water. Organic layers were dried over Na₂SO₄ and solvent was evaporated yielding 1.3 g of white powder which was further purified by column chromatography (DCM:MeOH:NH₄OH 90:9:0.9) yielding 0.77 g of the title product as a white powder.

MS (ES+): 806.3 [MH]⁺

### Intermediate 1

### Procedure B2

### Step 1: Preparation of 8

Step 1, of Procedure B2 was performed in two separate batches each starting from 880 g of 9-deoxo-9a-methyl-9a-aza-9a-homoerythromycin A (2) using the below described procedure.

9-Deoxo-9a-methyl-9a-aza-9a-homoerythromycin A (2) (880 g, 1.18 mol) and DMF/DMA (710 mL, 5.32 mol), were stirred in DMF (1.32 l) at 70° C. overnight. A mini workup was carried out and NMR showed the reaction had gone to completion. The reaction vessel was cooled to −10° C. and water (6.2 L) added slowly over 30 minutes causing product precipitation. The reaction was allowed to warm to room temperature and stirred for 2 hours. The white solid was collected by filtration and washed with water (3.7 L). ¹H NMR showed DMF still present 5-10%, therefore the reaction was further slurried with water (2.5 L) for 1 hour, the white solid collected by filtration and dried under vacuum at 50° C. for 3 days yielding title product (708 g, batch 1) as a white solid.

The aqueous filtrates were combined and extracted with ethyl acetate (6 L). The ethyl acetate was washed with water (1 L), dried over Na₂SO₄ and the solvent removed to afford additional amount of title product (99 g of product, in which according to ¹H NMR 74 g of title product (8), 12% DMF and 18% ethyl acetate was present), giving in total 782 g (83%) of title product from batch 1, Step 1.

From batch 2, Step 1, Procedure B2 in total 863 g of title product (8) was obtained.

Two batches from Step 1, Procedure B2 were combined giving 1635 g of title product (8) which was used in the next step without any further purification.

### Step 2: Preparation of 9

Step 2, of Procedure B2 was performed in 2 separate batches (batch 1 starting from 800 g and batch 2 starting from 835 g of compound 8 from step 1, of Procedure B2, using the procedure described below.

To a solution of compound 8 from step 1 (800 g, 0.99 mol) in DCM (16.3 L), DMAP (12.7 g, 0.1 mol) and triethylamine (865 mL, 6.2 mol) were added. Solution was cooled to −6° C. under nitrogen and then acetic acid anhydride (510 mL, 5.4 mol) was added dropwise maintaining temperature below 0° C. After stirring overnight, TLC showed the reaction was complete. The batch was split into two equal portions and each portion washed with sodium bicarbonate (2×2.1 L). The organics were dried over sodium sulfate (700 g), filtered and concentrated in vacuo. The white solid was dried under vacuum at 50° C. overnight to yield title product (9) (902 g, in which according to ¹H NMR 5% of DCM was present, MS data (MS (ES+) m/z: 888 [MH]+) conforms to the reference) form batch 1. From batch 2, Step 2, Procedure B2 1043 g of title product (9) was obtained (in which according to ¹H NMR ˜10% DCM and ˜5% DMF were present).

Two batches from Step 2, Procedure B2 were combined giving 1945 g of title product (9) which was used in the next step without any further purification.

### Step 3: Preparation of 10b

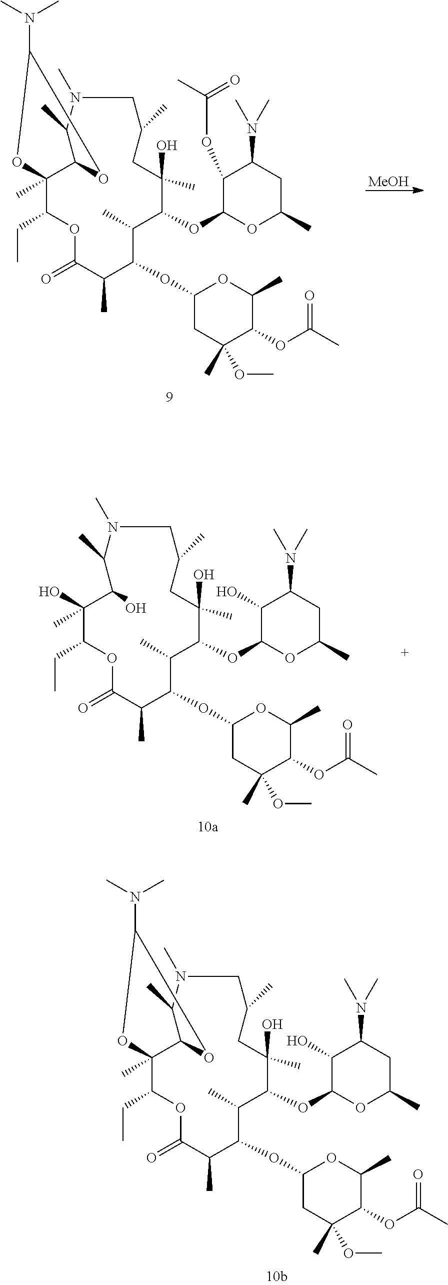

Step 3, of Procedure B2 was performed in 3 separate batches (batch 1 starting from 700 g, batch 2 starting from 664 g and batch 3 starting from 574 g of compound 9 from step 2, of Procedure B2, using the procedure described below.

Compound 9 from Step 2, Procedure B2 was stirred in MeOH (18 L) over four days at ˜22° C. Then, methanol was reduced in vacuo to approximately 3 L and the white precipitate collected by filtration and washed with methanol (1 L). The white solid was dried at 35° C. under vacuum overnight giving 415 g of the compound 10b from batch 1, which was used in step 4, procedure B2 without any further purification.

The filtrate was evaporated to give 254 g of a mixture of the protected compound 10b and partially deprotected compound 10a from batch 1.

From batch 2, Step 3, procedure B2 514 g of a mixture of the protected compound 10b and partially deprotected compound 10a and from batch 3, Step 3, procedure B2 510 g of a mixture of the protected compound 10b and partially deprotected compound 10a was obtained.

The mixed material (protected compound 10b and partially deprotected compound 10a from three batches from Step 3, Procedure B2 were combined giving in total 1278 g of material that was devided into two batches and submitted to the procedure described below.

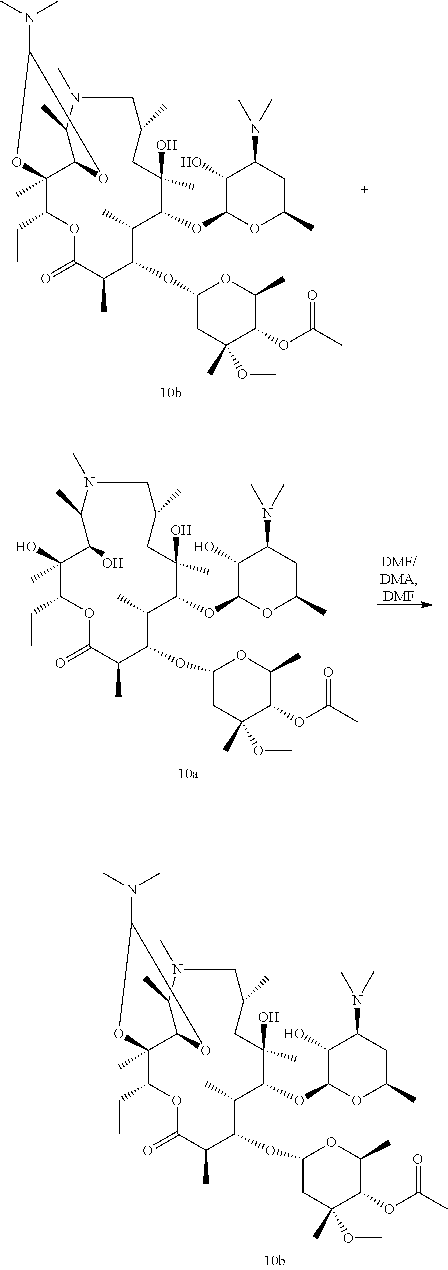

The mixture of unprotected 10a and protected compound 10b (560 g) was dissolved in DMF (840 mL), DMF-DMA (480 mL) was added and reaction mixture heated to 70° C. under N₂ for 6 h. Analysis by TLC showed the reaction to be complete. The reaction was poured into ice-water (7.4 kg) with vigorous stirring. After stirring for 2 hours at 0° C. the precipitate was filtered and washed with water (2 L). The product obtained was dried in a vacuum oven at 40° C. for 2 days to give 425.6 g of compound 10b as a white solid.

The aqueous filtrate was extracted with EtOAc (3×3.7 L) and the combined organics were then washed with water (2×3.7 L), dried (sodium sulfate), filtered, concentrated in vacuo, and dried in a vacuum oven at 40° C. for 2 days to give 123 g of compound 10b as a white solid. ¹H NMR indicated that both isolated solids were of similar purity and were comparable to the reference. Total yield of 10b from batch 1 was 548.6 g and from batch 2 was 763 g. The product was used in the next stage without further purification.

### Step 4: Preparation of 11b

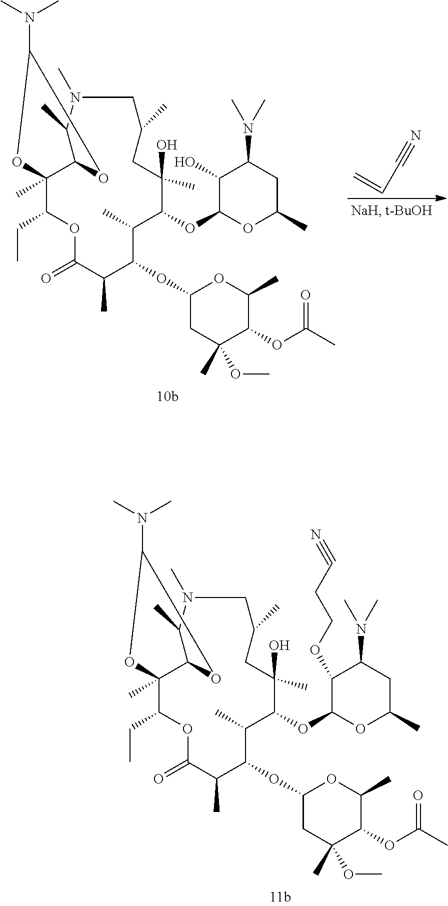

The reaction was performed in 18 separate batches starting from 1722 g of compound 10b obtained in Step 3, Procedure B2 using the procedure described below.

| | Amount of | |
|---|---|---|
| | Compound 10b | |
| Batch | (g) | Yield(g) |
| 1 | 96 | 94 |
| 2 and 3 | 192 | 192 |
| 4 | 96 | 99 |
| 5 and 6 | 192 | 183 |
| 7 | 96 | 97 |
| 8 and 9 | 192 | 187 |
| 10, 11 and 12 | 288 | 302 |
| 13, 14 and 15 | 288 | 308 |
| 16, 17 and 18 | 282 | 304 |

Sodium hydride (4.79 g, 60% dispersion in oil) was added in portions to a cooled (−6° C.) solution of acrylonitrile (654 mL) and t-butanol (47.3 mL) maintaining temperature less than 0° C. Compound 10b from Step 3, Procedure B2 (96.2 g) was then added portionwise maintaining temperature less than −5° C. over 15 minutes. The reaction was allowed to warm to 0° C. and after 20 minutes TLC indicated consumption of starting material. After 65 minutes at 0° C. acetic acid (7.5 mL) was added. IPA (200 mL) was used to transfer the reaction mixture to an evaporation flask and the reaction was concentrated in vacuo. IPA (300 mL) was added and the reaction concentrated in vacuo. The residue was slurried in EtOAc (1.5 L) for 30 minutes and then filtered (removal of polymer) and the filter cake washed with further EtOAc (1.2 L). The filtrate was concentrated in vacuo to a volume of 540 mL and the resultant cloudy solution was washed with water (2×180 mL). The aqueous extracts were combined and washed with EtOAc (2×180 mL) and the organics were combined, dried over sodium sulfate (100 g) and filtered. The organics were concentrated in vacuo to give crude product. Then, DCM (87 mL) was added to the crude product to give a cloudy solution, which added heptane (1440 mL) was added. After slurrying for 10 minutes at 7° C. the resultant suspension was filtered through Celite (11 g). The filter cake was washed with DCM/heptane (72 mL DCM in 1220 mL heptane). The filtrate was concentrated in vacuo and dried under high vacuum to give 93.5 g (91%) of compound 11b which was used in the next step without further purification.

### Step 5: Preparation of 12

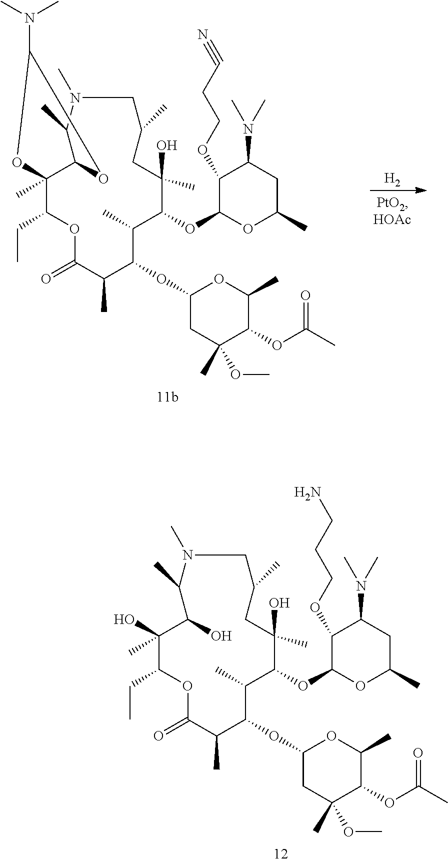

Step 5, of Procedure B2 was performed in 2 separate batches (batch 1 starting from 826 g and batch 2 starting from 934 g of compound 11b from step 4, of Procedure B2) using the procedure described below.

A solution of compound 11b obtained in Step 4, Procedure B2 (826 g) in acetic acid (6.32 L) was added to platinum (IV) oxide (80.4 g) and the resultant suspension was stirred at a hydrogen pressure of 5 bar overnight. The catalyst was removed by filtration and water (7 L) was added. The pH of the mixture was adjusted to 9.5 by addition of 40% aqueous sodium hydroxide solution (˜10 L). The mixture was then extracted with DCM (4×5 L then 2×3 L). The combined organics were washed with brine (2 L) and then dried (sodium sulfate, 800 g) and filtered. The organics were then concentrated in vacuo and dried in a vacuum oven at 40° C. overnight to give the 627 g of compound 12 from batch 1 as a white solid.

From batch 2, step 5, procedure B2 890 g of compound 12 was obtained.

The product was used in the next stage without further purification.

### Step 6: Preparation of Intermediate 1

### 2′-O-(3-Aminopropyl)-9-deoxo-9a-methyl-9a-aza-9a-homoerythromycin A

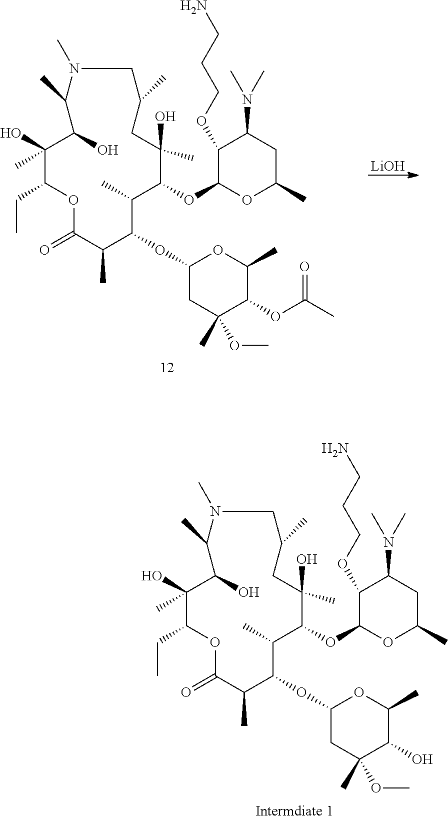

Step 6, of Procedure B2 was performed in four separate batches.

### Step 6, Batch A:

Compound 12 from step 5, Procedure B2 (313.5 g) in THF (3.17 L) and 0.5 M LiOH (2.48 L) was heated at 64° C. for 3 days. The reaction was then cooled and concentrated in vacuo to a volume of about 3 L. The product was extracted with EtOAc (4.1 L, then 3×2.1 L). The combined organics were washed with saturated brine (1.3 L). The organics were dried (sodium sulfate, 1.1 kg) filtered, concentrated in vacuo and then oven dried to give crude Intermediate 1 (245.9 g).

### Step 6, Batch B:

Compound 12 from step 5, Procedure B2 (313.5 g) in THF (3.17 L) and 0.5 M LiOH (2.48 L) was heated at 64° C. for 3 days. Analysis by LCMS indicated starting material remained. The reaction was heated at 64° C. overnight. Analysis by LCMS indicated starting material remained. Additional THF (317 mL) and 0.5 M LiOH (248 mL) were added and the reaction was heated at 64° C. for 4 hours by which time only a trace of starting material remained. The reaction was heated overnight and was then cooled and concentrated in vacuo to a volume of about 3 L. The product was extracted with EtOAc (4.1 L, then 3×2.1 L). The combined organics were washed with saturated brine (1.3 L). The organics were dried (sodium sulfate, 1.0 kg) filtered, concentrated in vacuo and then oven dried to give crude Intermediate 1 (232.5 g).

### Step 6, Batch C:

Compound 12 from step 5, Procedure B2 (445 g) in THF (4.5 L) and 0.5 M LiOH (3.5 L) was heated at 64° C. for 3 days. The reaction was then cooled and concentrated in vacuo to a volume of about 4 L. The product was extracted with EtOAc (4.9 L, then 3 L then 2 L then 1 L). The combined organics were washed with saturated brine (2 L). The organics were dried (sodium sulfate, 1.2 kg) filtered, concentrated in vacuo and then oven dried to give crude Intermediate 1 (336.8 g).

### Step 6, Batch D:

Compound 12 from step 5, Procedure B2 (445 g) in THF (4.5 L) and 0.5 M LiOH (3.5 L) was heated at 64° C. for 3 days. The reaction was then cooled and concentrated in vacuo to a volume of about 4 L. The product was extracted with EtOAc (4.9 L, then 3 L then 2 L then 1 L). The combined organics were washed with saturated brine (2 L). The organics were dried (sodium sulfate, 1.2 kg) filtered, concentrated in vacuo and then oven dried to give crude Intermediate 1 (296.5 g).

The 1027.1 g of crude Intermediate 1 was purified by column chromatography.

### Step 6, Purification 1:

Crude product (478.4 g) was purified by column chromatography (9.6 kg silica, eluent DCM/MeOH/ammonia, 900:60:10→900:66.9:11.5→900:75:12.5→900:83.3:13.7→900:90:15) to give pure Intermediate 1 (191.5 g) as well as product containing impurities with significantly higher R_{f} (70.4 g), product containing impuritiies with higher Rf (36.8 g) and product containing a close running lower R_{f} spot (52.6 g).

### Step 6, Purification 2:

Crude product (548.7 g) was purified by column chromatography (9.6 kg silica, eluent DCM/MeOH/ammonia, 900:60:10→900:66.9:11.5→900:75:12.5→900:83.3:13.7→900:90:15) to give pure Intermediate 1 (169.4 g) as well as product containing impurities with higher R_{f} (183.2 g) and product containing a close running lower R_{f} spot (30.3 g).

### Step 6, Repurification A:

Fractions containing product and a close running lower R_{f} spot (52.6 g+30.3 g) were purified by column chromatography (5.0 kg silica, 60.3 eq, eluent DCM/MeOH/ammonia, 900:90:15) to give pure Intermediate 1 (8.3 g) and mixed fractions (51.1 g).

### Step 6, Repurification B:

The combined material containing higher R_{f} impurities (220 g) and remaining crude Step 6 material (75 g) was purified by column chromatography (10 kg silica, 33.9 eq, eluent DCM/MeOH/ammonia, 900:60:10→900:66.9:11.5→900:75:12.5→900:83.3:13.7→900:90:15→900:180:30→0:90:10) to give pure Intermediate 1 (134.5 g) and material containing significant higher R_{f} impurities (124.0 g).

All of the pure material obtained was further dried to give a total of 431.9 g of Intermediate 1. This material was blended. The material was dissolved in DCM (2 L), dried with sodium sulfate (100 g), filtered and the filter cake washed with DCM (1 L). The filtrate was concentrated in vacuo. The material was ground up to a fine powder and dried to give 430.0 g of Intermediate 1.

### Intermediate 1

### Procedure B3

### Step 1: Preparation of 8

Step 1, of Procedure B3 was performed in two separate batches each starting from 1500 g of 9-deoxo-9a-methyl-9a-aza-9a-homoerythromycin A (2) using the below described procedure.

9-Deoxo-9a-methyl-9a-aza-9a-homoerythromycin A (2) (1500 g, 2.0 mol) and DMF/DMA (1.21 L, 9.1 mol), were stirred in DMF (2.25 l) at 70° C. overnight. A mini workup was carried out and NMR showed the reaction had gone to completion. The reaction vessel was cooled to 10° C. and water (14 L) added slowly causing product precipitation. The reaction was allowed to warm to room temperature and stirred for 2 hours. The white solid was collected by filtration and washed with water (3. L). The solid was dried under vacuum for 6 days yielding 1443.5 g of title product as a white solid (purity>95%, excluding solvents, 1393.1 g of title product (8) was present).

The aqueous filtrates were combined and extracted with ethyl acetate (10 L). The ethyl acetate was washed with water (2 L), dried over Na₂SO₄ and the solvent removed to afford additional amount of title product (142.4 g of product, in which excuding solvents 131.3 g of title product (8) was present) giving in total 1520.5 g of title product from batch 1, Step 1.

From batch 2, Step 1, Procedure B3 in total 1558.7 g of title product (8) was obtained. Two batches from Step 1, Procedure B3 were combined giving 3079.2 g of title product (8) which was used in the next step without any further purification.

### Step 2: Preparation of 9

Step 2, of Procedure B3 was performed in 4 separate batches starting from 811.1 g, 726.7 g, 757.7 g and 784.3 g of compound 8 from step 1, of Procedure B3, using the procedure described below.

To a solution of compound 8 from step 1 (784.3 g) in DCM (16 L), DMAP (11.9 g) and triethylamine (848 mL, 6.2 mol) were added. Solution was cooled to −6° C. under nitrogen and then acetic acid anhydride (499 mL) was added dropwise maintaining temperature below 0° C. After stirring overnight, TLC and MS showed the reaction was complete. The batch was washed with sodium bicarbonate (3×2.8 L). The organics were dried over sodium sulfate, filtered and concentrated in vacuo. The white solid was dried under vacuum at 50° C. overnight to yield 908 g of title product (9) in which according to ¹H NMR 851.3 g of active title product (9) was present.

From batches 2, 3 and 4 Step 2, Procedure B3, 870.1 g, 805.7 g and 835.6 g of title product (9) was obtained.

4 batches from Step 2, Procedure B3 were combined giving 3362.7 g of title product (9) which was used in the next step without further purification.

### Step 3: Preparation of 10b

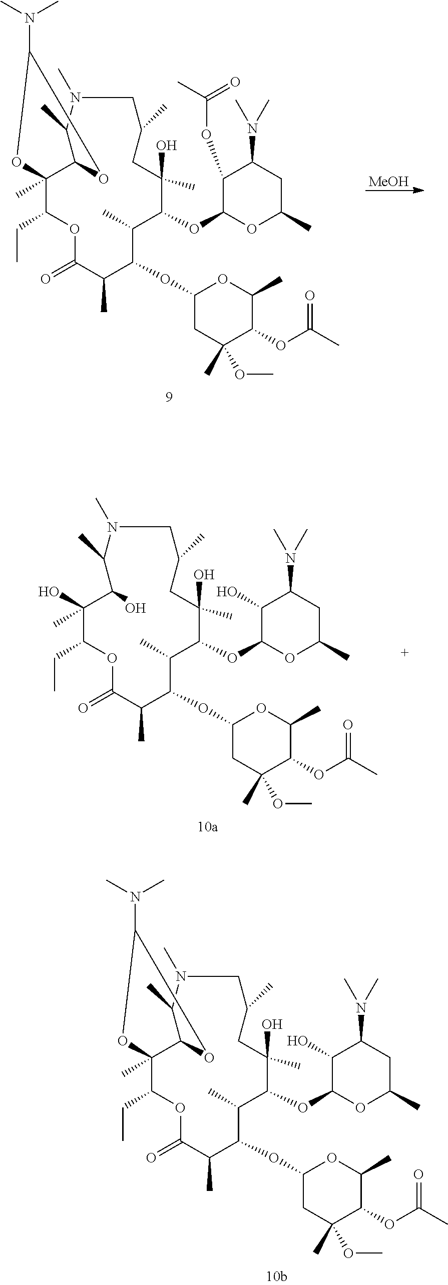

Step 3, of Procedure B3 was performed in 7 separate batches (starting from 4×700 g, 2×350 g and 1×97 g of compound 9 from step 2, of Procedure B3, using the procedure described below.

Compound 9 from Step 2, Procedure B3 (350 g) was stirred in MeOH (9 L) over five days at ˜24° C. Then, methanol was reduced in vacuo to approximately 1-1.5 L and the white precipitate collected by filtration and washed with methanol (0.2 L). The white solid was dried at 35° C. under vacuum overnight and then at 40° C. under vacuum overnight giving 182.7 g of the mixture of compound 10a and 10b (˜23% of 10a in mixture).

The filtrate was evaporated, dried at 35° C. overnight and then at 40° C. overnight to give 134.4 g of a mixture of 10a and 10b (−98% of 10a in mixture).

From other batches of Step 3, procedure B3a mixtures of compound 10a and 10b was obtained as stated in table below.

| | | Mass of product |
|---|---|---|
| Batch | Mass of input | (solid + filtrate) |
| 1 | 700 g | 375.3 g + 288.8 g |
| 2 | 700 g | 344.6 g + 322.4 g |
| 3 | 350 g | 182.7 g + 134.4 g |
| 4 | 700 g | 366.6 g + 287.0 g |
| 5 | 700 g | 400.9 g + 255.2 g |
| 6 | 350 g | 167.6 g + 156.7 g |
| 7 | 97 g | 56.6 g + 32.2 g |

Mixtures of compounds 10a and 10b originated from solid containing ˜10-25% of deprotected compound 10a were combined giving 1894.3 g.

Mixtures of compounds 10a and 10b originated from filtrate containing ˜75-98% of deprotected compound 10a were combined giving 1476.7 g.

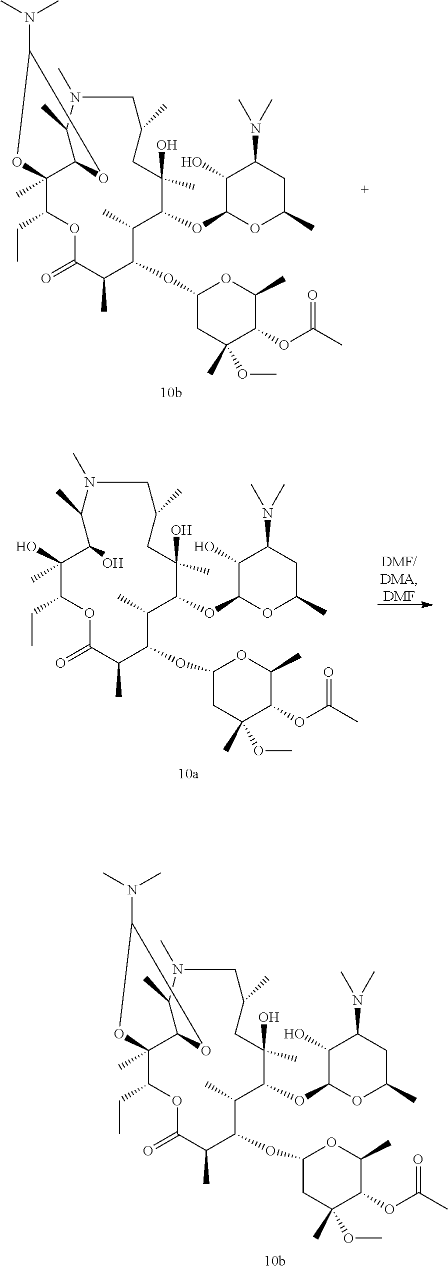

The mixture of unprotected 10a and protected compound 10b (1894.3 g) was dissolved in DMF (2840 mL), DMF-DMA (1625 mL) was added and reaction mixture heated to 70° C. under N₂ for 6 h. Analysis by TLC showed the reaction to be complete. The reaction was poured into ice-water (26.4 kg) with vigorous stirring. After stirring for 1.5 hours at 0° C. the precipitate was filtered and washed with water (4 L). The product obtained was dried in a vacuum oven at 40° C. for several days (until constant mass) giving 1226.8 g of compound 10b as a white solid in which according to ¹H NMR 1221.8 g of active title product (10b) was present.

The aqueous filtrate was extracted with EtOAc (3×9.7 L) and the combined organics were then washed with water (2×10 L), dried (sodium sulfate), filtered, concentrated in vacuo, and dried in a vacuum oven at 40° C. for several days (until contant mass) giving 592.2 g of compound 10b as a white solid (561.9 g of active title product 10b).

¹H NMR indicated that both isolated solids were of similar purity and were comparable to the reference. Total yield of 10b from batch 1 was 1783.7 g and from batch 2 was 1369.8 g. The product was used in the next stage without further purification.

### Step 4: Preparation of 11b

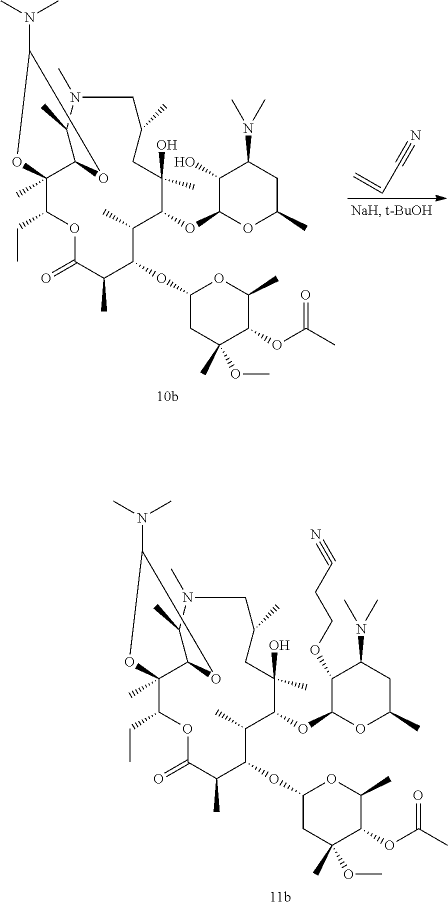

The reaction was performed in 22 separate batches starting from compound 10b obtained in Step 3, Procedure B3, using the procedure described below. The quantity of each batch is stated in table below.

| Batches | Input(g) | Yield(g) |
|---|---|---|
| 1-8 | 8 × 144 g | 1348.3 g |
| 9, 10, 11, 13 | 2 × 144 g | 618.2 g |
| | 2 × 145 g | |
| 12 | 145 g | 136.5 g |
| 14, 15, 20 | 3 × 145 g | 470.6 g |
| 16, 17, 18, 19, 21, 22 | 6 × 145 g | 968.5 g |
| | 1 × 28 g | |

Sodium hydride (7.2 g, 60% dispersion in oil) was added in portions over 5 minutes to a cooled (−10° C.) solution of acrylonitrile (980 mL) and t-butanol (71 mL) maintaining temperature less than −8° C. Compound 10b from Step 3, Procedure B3 (144.1 g) was then added portionwise maintaining temperature less than −5° C. over 10 minutes. The reaction was allowed to warm to 0° C. and after 40 minutes TLC indicated consumption of starting material. After 1 hour acetic acid (11.3 mL) was added. IPA (250 mL) was used to transfer the reaction mixture to an evaporation flask and the reaction was concentrated in vacuo. IPA (2×250 mL) was added and the reaction concentrated in vacuo. The residue was slurried in EtOAc (2 L) for 30 minutes and then filtered (removal of polymer) and the filter cake washed with further EtOAc (2×150 mL). The filtrate was concentrated in vacuo resulting in a foam.

The filter cake was then slurried in further EtOAc (810 mL) and filtered. The filtrate was combined with the foam and the resulting cloudy solution was washed with water (2×270 mL). The aqueous extracts were combined and extracted with EtOAc (2×270 mL) and the organics were combined, dried (sodium sulfate, 200 g) and filtered. The organics were concentrated in vacuo to give crude product. DCM (130 mL) was added to the crude product to give a cloudy solution, to which heptane (2160 mL) was added. After slurrying for 10 minutes at room temperature, the resultant suspension was filtered through Celite (100 g). The filter cake was washed with DCM/heptane (110 mL DCM in 1830 mL heptane). The filtrate was combined with filtrate from 2 other batches and was concentrated in vacuo to give crude product as an oil (644 g). A portion of this material (32 g) was purified by chromatography (SiO₂ 213 g, eluent=DCM→90:10:0.3 DCM/MeOH/ammonia) to give 24 g of title compound 11b with polymer removed. The remaining crude product (612 g) was combined with crude product of similar purity from 5 other batches (997 g) and was purified by chromatography (Si0₂ 6 kg, eluent=10% DCM in heptane→→→90:10:0.3 DCM/MeOH/ammonia). The bulk of the higher running polymer impurity was removed and product containing fractions were concentrated in vacuo to give 1348.3 g of title product 11b as a viscous oil containing approximately 10-15% polymer. The product was used in the next stage without further purification.

For batches 9-22 the same procedure were applied with the difference that product was not purified by chromatography but used in the next stage without further purification.

### Step 5: Preparation of 12

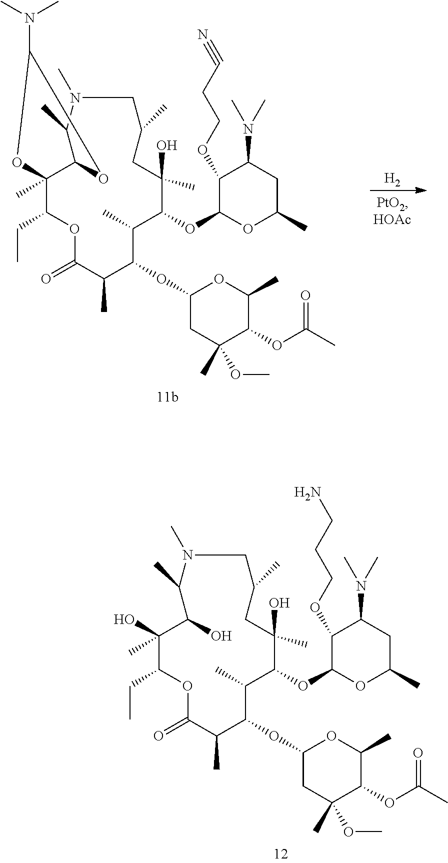

Step 5, of Procedure B3 was performed in 4 separate batches (starting from 107 g, 1710.5 g, 483.3 g and 1264.1 g of compound 11b from step 4, of Procedure B3) using the procedure described below.

A solution of compound 11b obtained in Step 4, Procedure B3 (1710.5 g) in acetic acid (13 L) was added to platinum (IV) oxide (150 g) and the resultant suspension was stirred at a hydrogen pressure of 5 bar overnight. The catalyst was removed by filtration and methanol (3.5 L) was added and filtrated The filtrate was concentrated in vacuo to a mass of 4072 g and water (14.5 L) and DCM (10 L) were added. After stirring for 5 minutes the phases were separated. The pH of the aqueous phase was adjusted to ˜9 by addition of 40% aqueous sodium hydroxide solution (2.5 L). The aqueous was then extracted with DCM (2×10 L, 1×1 L, 2×5 L and 4×2.5 L). The combined organic extracts (not including the initial DCM wash) were washed with brine (4.1 L) and then dried (sodium sulfate, 800 g) and filtered. The organics were then concentrated in vacuo and dried in a vacuum oven at 40° C. overnight to give title product 12 (1571.9 g) as a white solid which was used in the next stage without further purification.

From other 3 batches additional 1508 g of title product 12 was obtained.

### Step 6: Preparation of Intermediate 1

### 2′-O-(3-Aminopropyl)-9-deoxo-9a-methyl-9a-aza-9a-homoerythromycin A

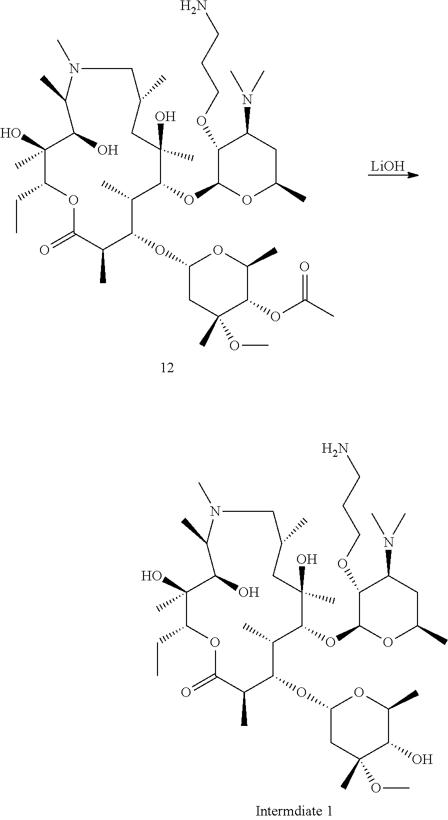

Step 6, of Procedure B3 was performed in 8 separate batches using the procedure described below. The quantity of each batch is stated in table below.

| | | |
|---|---|---|
| | | Mass of crude |
| Batch | Mass of input | product |

| | | | |
|---|---|---|---|
| Use test | 63 | g | 42 g |
| 1 | 364.9 | g | 288.3 g |
| 2 | 450 | g | 323.2 g |
| 3 + 4 | 2 × 450 | g | 656.6 g |
| 5 + 6 | 2 × 450 | g | 598.8 g |
| 7 | 268.1 | g | 180.7 g |
| 8 | 132 | g | 99.7 g |

Compound 12 from step 5, Procedure B3 (450 g) in THF (4.5 L) and 0.5 M LiOH (3.5 L) was heated at 64° C. for 3 days. The reaction was then cooled and concentrated in vacuo to a volume of about 4 L. At this stage the material was combined with a reaction carried out on the same scale. The product was extracted with EtOAc (1×11.8 L, 1×6 L and 3×4 L). The combined organics were washed with saturated brine (4 L). The organics were dried (sodium sulfate, 3.4 kg) filtered, concentrated in vacuo and then oven dried to give crude Intermediate 1 (656.6 g).

The 2189.3 g of crude Intermediate 1 was purified by column chromatography at 5 columns.

The purification was repeated 4 times each with 500 g of the crude product by column chromatography (10 kg silica, eluent DCM/MeOH/ammonia, 900:60:10→900:66.9:11.5→900:75:12.5→900:83.3:13.7→900:90:15) to give purified material (56.3 g 97.4 g, 145.3 g and 112.3 g respectively) as well as mixed fractions.

The rest of the crude product (189 g) was purified by column chromatography (5 kg silica, eluent DCM/MeOH/ammonia, 900:60:10 to give purified material (60.4 g) as well as mixed fractions.

Mixed fractions from all 5 columns were combined and concentrated as follows:

Fractions containing product and higher close running R_{f} impurity were concentrated to give 256.5 g of product ˜80% pure by ¹H NMR.

Fractions containing product and lower close running R_{f} impurity were concentrated to give 331 g of product ˜80% pure by ¹H NMR.

Fractions containing product and lower running R_{f} impurity were concentrated to give 182.8 g.

### Repurification A

Fractions containing product and higher close running R_{f} impurity (256.5 g) was further purified by column chromatography (7 kg silica, eluent DCM/MeOH/ammonia, 900:45:7.5→900:60:10→900:90:15→800:180:20) to give title product (109 g) as well as mixed fractions (product having higher R_{f} impurity (10.6 g); product having lower R_{f} impurity (23.6 g)).

Product having higher R_{f} impurity (10.6 g) was slurried overnight in acetonitrile (20 mL), filtered and washed with acetonitrile (10 mL) giving 5.8 g of title product.

Product having lower R_{f} impurity (23.6 g) was slurried overnight in acetonitrile (50 mL), filtered and washed with acetonitrile (25 mL) and dried to give 5 g of title product.

### Repurification B:

Fractions containing product and lower close running R_{f} impurity (331 g) was slurried overnight in acetonitrile (660 mL), filtered, washed with acetonitrile (200 mL) and dried to give 213 g of title product.

### Repurification C:

The product containing lower R_{f} impurity (182.8 g) was slurried overnight in acetonitrile (160 mL), filtered, washed with acetonitrile (180 mL) giving 126 g of material which was further slurried in acetonitrile (250 mL), filtered and washed with acetonitrile (150 mL) to give 104.4 g of title product.

### Repurification D:

The combined, dried single spot product from columns 1 and 2 (153 g) was slurried overnight in acetonitrile (300 mL) and then filtered and washed with acetonitrile (80 mL) to give ˜134.4 g of title product.

All of the pure material obtained was further dried to give a total of 842.9 g of Intermediate 1. This material was blended. The material was dissolved in DCM (7 L), filtered and the filter cake washed with DCM (1.2 L). The filtrate was concentrated in vacuo. After being ground and being subjected to extended drying at 40° C., a total of 777.2 g of title product, Intermediate 1 was obtained.

### EXAMPLES

### General Procedure for Examples 1 and 3 to 6

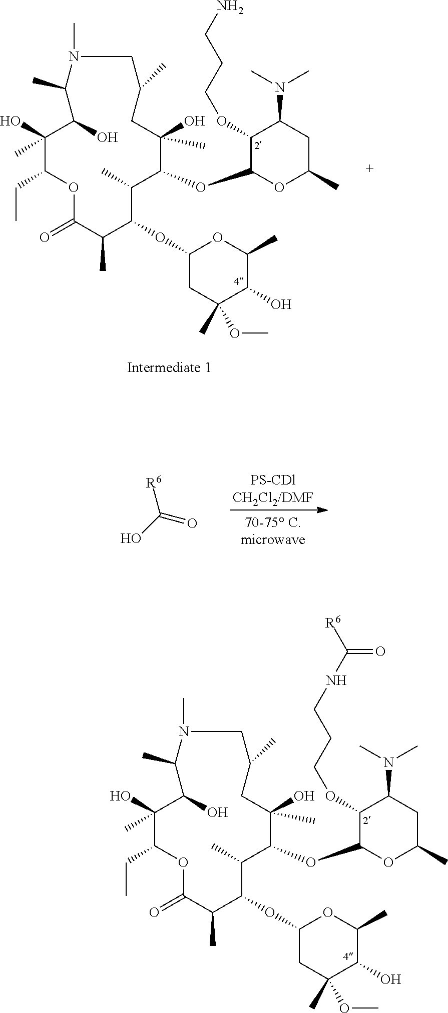

Dry PS-Carbodiimide resin (PS-CDI, loading: 1.2 mmol/g, 1.3 eq) was added to a reaction vessel. The corresponding acid (1.05 eq) and HOBt (0.7 eq), dissolved in a dry mixture of DCM (1 mL) and DMF (100-200 μL), were added to the resin. The mixture was stirred at room temperature for 5 minutes upon which 2′-O-(3-Aminopropyl)-9-deoxo-9a-methyl-9a-aza-9a-homoerythromycin A, Intermediate 1 dissolved in dry DCM (1 mL) was added. The reaction mixture was heated by microwave irradiation (300 W, ramp time 2.00 min) at 70-75° C. for 6-7 minutes.

HOBt was scavenged using PS-trisamine (loading: 4.11 mmol/g, 5 equivalent according to HOBt) for 3 hours at room temperature. The resin was removed from the reaction mixture by filtration and washed with DCM (1-1.5 mL). Evaporation of the solvent afforded corresponding compound of examples 1 and 3 to 6.

### Example 1

### 2′-O[3-(Acetylamino)propyl]-9-deoxo-9a-methyl-9a-aza-9a-homoerythromycin A

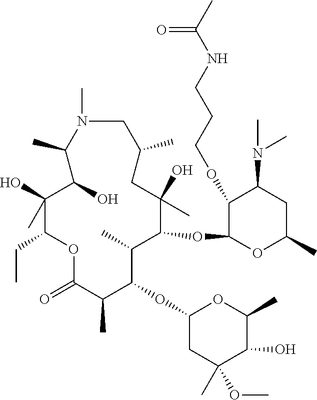

According to the general procedure for Examples 1 and 3 to 6 starting from 2′-O-(3-aminopropyl)-9-deoxo-9a-methyl-9a-aza-9a-homoerythromycin A, Intermediate 1 (30 mg, 0.037 mmol) and acetic acid (1.05 equiv), title compound (30.4 mg) was obtained.

MS (ES+) m/z: 848.40 [MH]+

¹³C (DMSO, 125 MHz) δ 177.4, 169.0, 102.4, 94.8, 82.7, 80.1, 77.7, 77.5, 76.7, 75.3, 74.0, 73.2, 72.9, 70.3, 69.0, 67.2, 65.1, 64.3, 61.8, 49.2, 45.2, 42.2, 41.9, 41.2, 36.7, 36.1, 35.0, 31.7, 30.0, 27.8, 26.4, 23.1, 22.5, 21.8, 21.4, 21.3, 18.9, 18.1, 15.2, 11.3, 8.7, 7.2.

### Example 2

### 2′-O-[3-(Propanoylamino)propyl]-9-deoxo-9a-methyl-9a-aza-9a-homoerythromycin A

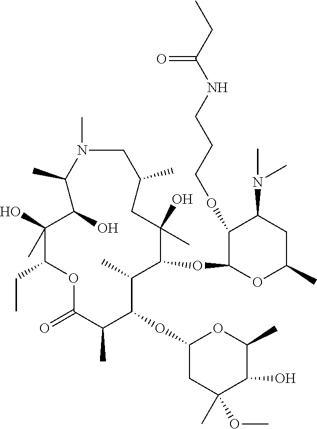

### Method A:

Dry PS-Carbodiimide resin (PS-CDI, loading: 1.2 mmol/g, 1.3 eq) was added to a reaction vessel. Propanoic acid (1 eq) and HOBt (0.7 eq), dissolved in a dry mixture of DCM (2.4 mL) and DMF (300 μL), were added to the resin. The mixture was stirred at room temperature for 5 minutes upon which 2′-O-(3-Aminopropyl)-9-deoxo-9a-methyl-9a-aza-9a-homoerythromycin A, Intermediate 1 (30 mg, 0.037 mmol) dissolved in dry DCM was added. The reaction mixture was heated by microwave irradiation (300 W, ramp time 2.00 min) at 70° C. for 5 minutes.

HOBt was scavenged using PS-trisamine (loading: 4.11 mmol/g, 5 equivalent according to HOBt) for 3 hours at room temperature. The resin was removed from the reaction mixture by filtration. Evaporation of the solvent afforded title product (30.8 mg).

MS (ES+) m/z: 862.73 [MH]+

¹³C (DMSO, 125 MHz) δ 177.5, 172.7, 102.4, 94.7, 82.7, 80.1, 77.7, 77.5, 76.7, 75.3, 74.0, 73.2, 72.9, 70.4, 67.2, 65.1, 64.3, 61.8, 49.2, 45.2, 42.2, 41.9, 41.2, 36.6, 36.1, 35.0, 31.8, 30.1, 29.0, 27.8, 26.4, 22.5, 21.8, 21.4, 21.3, 18.9, 18.0, 15.2, 11.3, 10.4, 8.7, 7.

### Method B:

To the solution of propanoic acid (1.160 mL, 15.51 mmol) in DCM (200 ml), TEA (10 mL), 1-hydroxybenzotriazole hydrate (2.179 g, 16.13 mmol) and solution of EDC×HCl (4.04 g, 21.09 mmol) in DCM (100 ml) were added and reaction mixture was stirred under N₂ for 15 minutes. Solution of the 2′-O-(3-aminopropyl)-9-deoxo-9a-methyl-9a-aza-9a-homoerythromycin A, Intermediate 1, (10 g, 12.41 mmol) in DCM (100 ml) was added, reaction mixture stirred at room temperature under N₂ over night, and saturated aueous NaHCO₃ (400 ml) was added. Layers were separated and aqueous layer was extracted with DCM (1×150 ml). Combined DCM layers were evaporated in vacuum.

The foamy white solid was dissolved in EtOAc (80 ml), H₂O (60 ml) was added and pH was adjusted to 4 and layers separated. To the H₂O layer DCM was added (60 ml), and pH was adjusted to 6 and layers separated. To the DCM layer, H₂O (40 ml) was added and pH adjusted to 9.5. Layers were separated and DCM evaporated in vacuum yielding 1.7 g of crude product which was precipitated from acetone/H₂O, dried at 50° C. in vacuum yielding 1.44 g of title product.

H₂O layer from the extraction on pH 6 was extracted with DCM (100 ml) at pH 6.3. and layers separated. To the DCM layer H₂O (50 ml) was added, pH was adjusted to 9.5, layers separated and DCM evaporated in vacuum yielding additional 5.3 g of crude product.

H₂O layer from the extraction on pH 6.3 was extracted with DCM (100 ml) and layer separated. To the DCM layer H₂O (50 ml) was added, pH was adjusted to 9.5, layers separated and DCM evaporated in vacuum yielding additional 2.45 g of crude product. Crude products from last two extractions were combined (7.75 g) and precipitated from acetone (5 ml)/H₂O (50 ml). Precipitate was dried at 50° C. in vacuum yielding additional 4.38 g of title product.

Title product obtained by method B has identical mass and NMR data as product obtained by method A.

### Example 3

### 2′-O-{3-[(2-Methylpropanoyl)amino]propyl}-9-deoxo-9a-methyl-9a-aza-9a-homoerythromycin A

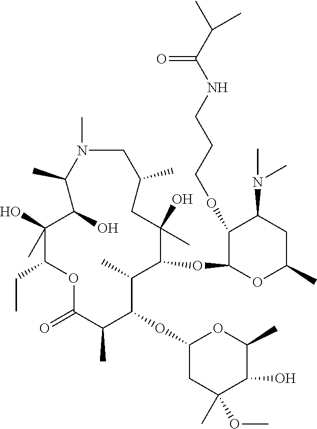

According to the general procedure for Examples 1 and 3 to 6 starting from 2′-O-(3-aminopropyl)-9-deoxo-9a-methyl-9a-aza-9a-homoerythromycin A, Intermediate 1 (30 mg, 0.037 mmol) and isobutanoic acid (1.05 equiv), title compound (26.0 mg) was obtained.

MS (ES+) m/z: 876.47 [MH]+

¹³C (DMSO, 125 MHz) δ 176.9, 168.4, 101.8, 94.2, 82.1, 79.6, 77.2, 76.9, 76.1, 74.7, 73.4, 72.6, 72.4, 70.0, 68.4, 66.6, 64.5, 63.7, 61.3, 48.6, 44.6, 41.6, 41.3, 40.6, 36.0, 35.5, 34.4, 33.9, 31.3, 29.6, 27.2, 25.8, 21.9, 21.2, 20.8, 20.7, 19.4, 19.3, 18.7, 18.3, 17.4, 14.6, 10.7, 8.1, 6.6.

### Example 4

### 2′-O-{3-[(2,2-Dimethylpropanoyl)amino]propyl}-9-deoxo-9a-methyl-9a-aza-9a-homoerythromycin A

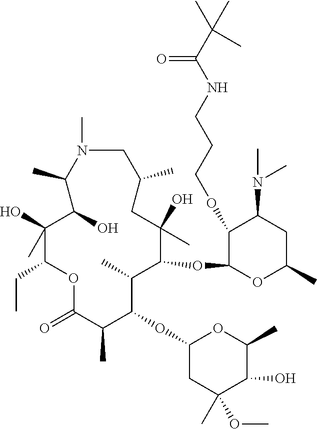

According to the general procedure for Examples 1 and 3 to 6 starting from 2′-O-(3-aminopropyl)-9-deoxo-9a-methyl-9a-aza-9a-homoerythromycin A, Intermediate 1 (30 mg, 0.037 mmol) and pivalic acid (1.05 equiv), title compound (28.4 mg) was obtained. MS (ES+) m/z: 890.50 [MH]+

¹³C (DMSO, 125 MHz) δ 177.5, 170.0, 102.4, 94.8, 82.5, 80.4, 77.7, 77.4, 76.7, 75.3, 74.0, 73.2, 72.9, 70.7, 69.0, 67.1, 65.1, 64.2, 61.9, 49.2, 45.2, 42.0, 41.9, 41.3, 38.3, 36.9, 36.1, 35.0, 30.4, 27.8, 27.2, 26.3, 22.5, 21.8, 21.4, 21.3, 18.9, 18.0, 15.2, 11.3, 8.7, 7.1.

### Example 5

### 2′-O-{3-[(N,N-Diethylglycyl)amino]propyl}-9-deoxo-9a-methyl-9a-aza-9a-homoerythromycin A

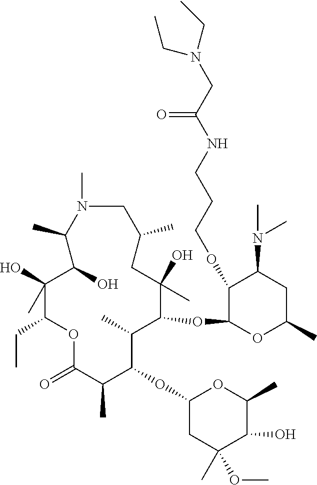

According to the general procedure for Examples 1 and 3 to 6 starting from 2′-O-(3-aminopropyl)-9-deoxo-9a-methyl-9a-aza-9a-homoerythromycin A, Intermediate 1 (102 mg, 0.127 mmol) and N,N-diethylglycine (1.05 equiv), title compound (83.0 mg) was obtained.

MS (ES+) m/z: 919.65 [MH]+

¹³C (DMSO, 125 MHz) δ 177.5, 170.8, 102.3, 94.7, 82.6, 80.2, 77.7, 77.4, 76.7, 75.2, 74.0, 73.2, 72.9, 69.9, 69.0, 67.2, 65.4, 65.1, 64.4, 62.0, 49.2, 48.2, 45.2, 42.0, 41.9, 41.3, 36.1, 35.8, 35.0, 32.3, 31.4, 30.7, 26.9, 26.4, 22.5, 21.8, 21.3, 18.9, 18.0, 15.1, 12.3, 11.3, 8.7, 7.2.

### Example 6

### 2′-O-{3-[(4-Pyridinylcarbonyl)amino]propyl}-9-deoxo-9a-methyl-9a-aza-9a-homoerythromycin A

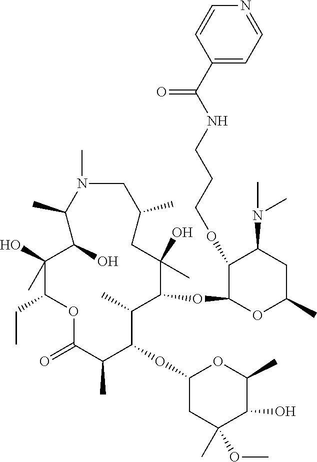

According to the general procedure for Examples 1 and 3 to 6 starting from 2′-O-(3-aminopropyl)-9-deoxo-9a-methyl-9a-aza-9a-homoerythromycin A, Intermediate 1 (30 mg, 0.037 mmol) and 4-pyridinecarboxylic acid (1.05 equiv), title compound (30.9 mg) was obtained.

MS (ES+) m/z: 911.30 [MH]+

¹³C (DMSO, 125 MHz) δ 176.9, 164.4, 150.0, 141.7, 121.0, 101.9, 94.2, 82.2, 79.8, 77.3, 77.0, 76.2, 74.7, 73.5, 72.7, 72.5, 70.1, 68.5, 66.7, 64.6, 63.7, 61.4, 48.7, 44.7, 41.6, 41.5, 40.7, 37.1, 35.6, 34.6, 31.6, 29.6, 27.3, 25.8, 21.9, 21.3, 20.9, 20.8, 18.4, 17.6, 14.7, 10.9, 8.3, 6.7.

### Example 7

### 2′-O-{3-[(N,N-Dimethylglycyl)amino]propyl}-9-deoxo-9a-methyl-9a-aza-9a-homoerythromycin A

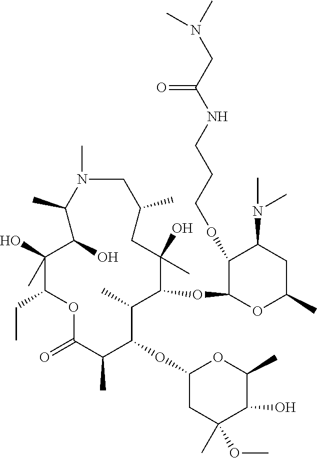

To the solution of 2′-O-(3-aminopropyl)-9-deoxo-9a-methyl-9a-aza-9a-homoerythromycin A, Intermediate 1 (500 mg, 0.62 mmol) in DCM (10 mL), N,N-dimethylglycine (1.25 equiv) and triethylamine (0.5 mL) were added and reaction mixture was stirred under N₂ for 15 min. To the reaction mixture HOBt (1.3 equiv) and solution of EDC×HCl (1.7 equiv) in DCM (25 mg/mL) were added and stirred at room temperature under N₂ over night. To the reaction mixture saturated aqueous NaHCO₃ (50 mL) was added, layers separated and aqueous layer extracted with DCM (20 mL). To the combined organic extracts water (30 mL) was added and pH adjusted to pH 6.5 with 2N HCl. Layers were separated, to the organic layer water was added and pH adjusted to pH 9.5 with NH₃:H₂O=1:1. Organic layer was evaporated under vacuum yielding title compound as a crude product, which was purified by column chromatography (eluent: EtOAc:n-hexane:Et₂N=100:100:20), to afford title compound (59 mg).

MS (ES+) m/z: 892.04 [MH]+

¹³C (DMSO, 125 MHz) δ 177.5, 169.5, 102.3, 94.8, 82.7, 80.1, 77.7, 77.5, 76.7, 75.3, 74.0, 73.2, 72.9, 70.1, 69.0, 67.2, 65.1, 64.4, 63.4, 61.8, 49.2, 45.8, 45.2, 42.2, 41.9, 41.1, 36.1, 36.0, 35.0, 31.6, 30.4, 27.8, 26.4, 22.5, 21.8, 21.4, 18.9, 18.0, 15.2, 11.3, 8.6, 7.2.

### Example 8

### 2′-O-{3-[(N-Methylglycyl)amino]propyl}-9-deoxo-9a-methyl-9a-aza-9a-homoerythromycin A

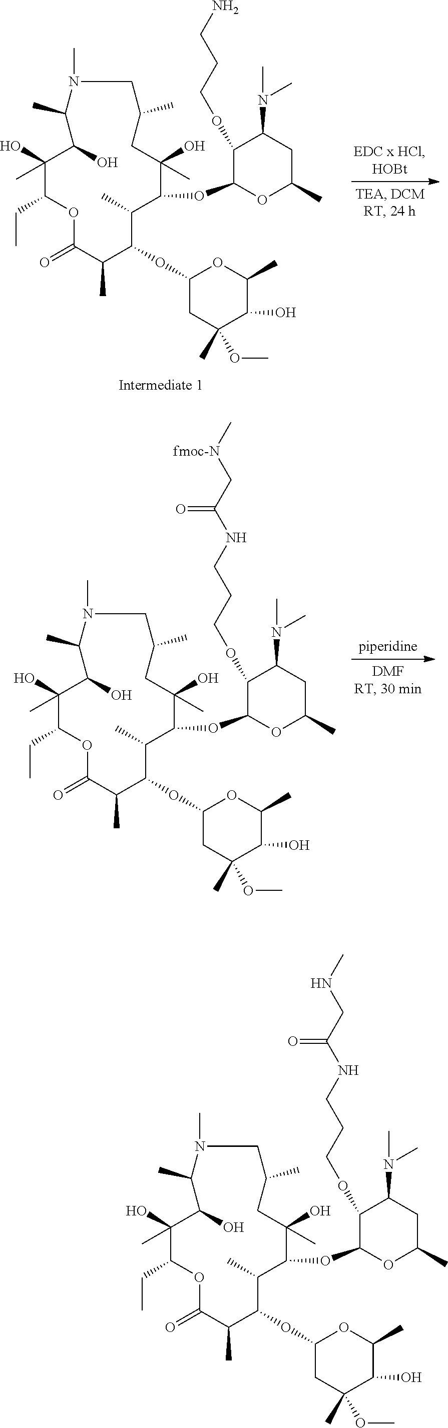

To a solution of 2′-O-(3-aminopropyl)-9-deoxo-9a-methyl-9a-aza-9a-homoerythromycin A, Intermediate 1 (500 mg, 0.62 mmol) in DCM (10 mL), Fmoc-sarcosine (241 mg, 0.775 mmol) and triethylamine (0.5 mL) were added and reaction mixture was stirred under N₂ for 15 min. To the reaction mixture HOBt (109 mg, 0.806 mmol) and solution of EDC×HCl (202 mg, 1.05 mmol) in DCM (10 mL) were added and the mixture stirred at room temperature under N₂ over night. Then, saturated aqueous NaHCO₃ (50 mL) was added, layers separated and aqueous layer extracted with DCM (20 mL). To the combined DCM extracts water (30 mL) was added and pH adjusted to pH 6.5. After separation of the layers, water (10 mL) was added to the organic layer and pH adjusted to 9.5. Layers were separated, organic layer was evaporated under vacuum yielding partially deprotected title product (506 mg), which was dissolved in DMF (5 mL), piperidine (1 mL) was added and the reaction mixture stirred for 30 min to compleate deprotection. Then, saturated aqueous NaHCO₃ (50 mL) and EtOAc (20 mL) were added and layers separated. To the EtOAc layer water (20 mL) was added and pH adjusted to 3.5. To aqueous layer DCM (10 mL) was added and pH adjusted to 6.6. After separation of the layers, DCM (10 mL) was added to aqueous layer and pH adjusted to 6.9. The layers were separated and the aqueous layer was re-extracted with DCM (4×10 mL). To the combined DCM layers water (20 mL) was added and pH adjusted to 9.5. Layers were separated, DCM layer was evaporated under vacuum yielding crude product (319 mg) which was precipitated from CH₃CN to yield title compound (237 mg).

MS (ES+) m/z: 878.04 [MH]+

¹³C (DMSO, 125 MHz) δ 177.5, 171.0, 102.3, 94.7, 82.7, 80.1, 77.7, 77.5, 76.7, 75.3, 74.0, 73.2, 72.9, 70.1, 69.0, 67.2, 65.1, 64.3, 61.8, 54.8, 49.2, 45.2, 42.2, 41.9, 41.2, 36.5, 36.1, 35.0, 31.8, 30.3, 27.8, 26.4, 22.5, 21.8, 21.4, 21.3, 18.9, 18.0, 15.2, 11.3, 8.7, 7.2.

### General Procedure for Examples 9 to 12

Dry PS-CDI resin (17.77 mg, 0.062 mmol) was added to a reaction vessel. The corresponding Fmoc-protected amino acid (0.062 mmol) and HOBt (66.5 mg, 0.434 mmol) dissolved in a mixture of dry DCM (1 mL) and dry DMF (200 μL) was added to the resin. The mixture was stirred at room temperature for 5 minutes upon which 2′-(3-aminopropyl)-9-deoxo-9a-methyl-9a-aza-9a-homoerythromycin A, Intermediate 1 (50 mg, 0.062 mmol), dissolved in dry DCM (1 mL) was added. The reaction mixture was stirred at room temperature for 18 hours. The resin was filtered off and washed with DCM (3×0.7 mL). The obtained reaction solution was concentrated, piperidine (100 μL, 1.01 mmol) was added and resulting mixture stirred at room temperature for 15 minutes. Then, water (5 mL) and DCM (2.5 mL) were added and pH adjusted to 3.5. Layers were separated and the aqueous layer washed with DCM (3×2.5 mL). Than, pH of aqueous layer was adjusted to 10.0 and aqueous layer was extracted with DCM (4×2 mL). The combined organic extracts at pH 10 were dried over anhydrous Na₂SO₄, filtered and concentrated to give the corresponding compound of example 9 to 12.

### Example 9

### 2′-O[3-(L-prolylamino)propyl]-9-deoxo-9a-methyl-9a-aza-9a-homoerythromycin A

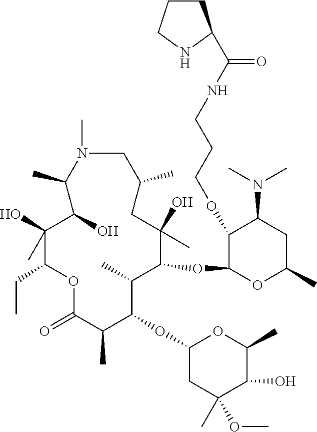

According to the general procedure for Examples 9 to 12 starting from 2′-O-(3-aminopropyl)-9-deoxo-9a-methyl-9a-aza-9a-homoerythromycin A, Intermediate 1 (50 mg, 0.062 mmol), and Fmoc-L-proline (20.93 mg, 0.062 mmol), title compound (48.8 mg) was obtained.

MS (ES+) m/z: 903.19 [MH]+.

### Example 10

### 2′-O-[3-(L-phenylalanylamino)propyl]-9-deoxo-9a-methyl-9a-aza-9a-homoerythromycin A

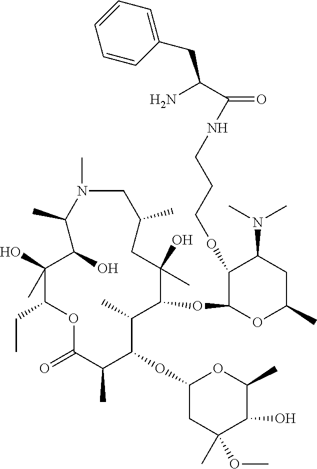

According to the general procedure for Examples 9 to 12 starting from 2′-O-(3-aminopropyl)-9-deoxo-9a-methyl-9a-aza-9a-homoerythromycin A, Intermediate 1 (50 mg, 0.062 mmol), and Fmoc-L-phenylalanine (24.03 mg, 0.062 mmol), title compound (52.1 mg) was obtained.

MS (ES+) m/z: 953.25 [MH]+.

### Example 11

### 2′-O-[3-(L-isoleucylamino)propyl]-9-deoxo-9a-methyl-9a-aza-9a-homoerythromycin A

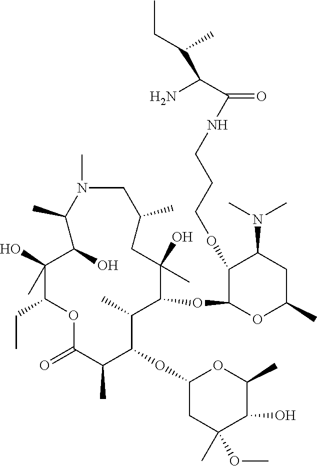

According to the general procedure for Examples 9 to 12 starting from 2′-O-(3-aminopropyl)-9-deoxo-9a-methyl-9a-aza-9a-homoerythromycin A, Intermediate 1 (50 mg, 0.062 mmol), and Fmoc-L-isoleucine (21.92 mg, 0.062 mmol), title compound (52.1 mg) was obtained.

MS (ES+) m/z: 919.24 [MH]+.

### Example 12

### 2′-O-[3-(L-methionylamino)propyl]-9-deoxo-9a-methyl-9a-aza-9a-homoerythromycin A

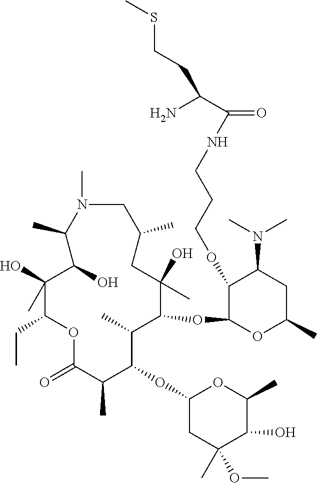

According to the general procedure for Examples 9 to 12 starting from 2′-O-(3-aminopropyl)-9-deoxo-9a-methyl-9a-aza-9a-homoerythromycin A, Intermediate 1 (50 mg, 0.062 mmol), and Fmoc-L-methionine (23.04 mg, 0.062 mmol), title compound (58 mg) was obtained.

MS (ES+) m/z: 937.27 [MH]+.

### Example 13

### 2′-O-(3-{[4-(Methyloxy)-4-oxobutanoyl]amino}propyl)-9-deoxo-9a-methyl-9a-aza-9a-homoerythromycin A

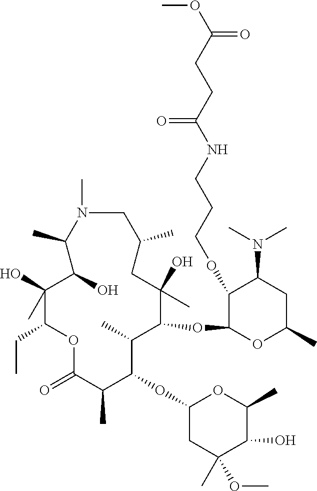

To the mono-methyl succinate (102 mg, 0.775 mmol), diluted in DCM (5 mL) under N₂ atmosphere, triethylamine (0.5 mL), HOBt (109 mg, 0.806 mmol) and solution of EDC (202 mg, 1.054 mmol) in DCM (6 mL) were added and reaction mixture was stirred at room temperature for 15 minutes. Then solution of 2′-O-(3-aminopropyl)-9-deoxo-9a-methyl-9a-aza-9a-homoerythromycin A, Intermediate 1 (500 mg, 0.620 mmol) in DCM (9 mL) was added and stirring continued at room temperature for 20 hours. Reaction mixture was extracted with saturated aqueous NaHCO₃ (2×15 mL). Combined aqueous layers were extracted with DCM (20 mL). Combined organic layers were evaporated in vacuum. White foamy solid was diluted in EtOAc (20 mL) and extracted with water (20 mL) at pH 4. Organic layer at pH 4 was discarded and aqueous extracted with DCM (20 mL) at pH 5.5. Organic layer at pH 5.5 was discarded and aqueous extracted with DCM (2×20 mL) at pH 6. To the combined organic layers at pH 6 water (20 mL) was added, pH adjusted to 9 and layers separated. DCM was evaporated in vacuum yielding crude product (360 mg) as a white foamy solid which was precipitated from EtOAc:n-hexane:diethyl-ether, dried at 50° C. for 5 hours yielding title compound (241 mg) as a white powder.

MS (ES+) m/z: 921.25 [MH]+.

¹³C (DMSO, 125 MHz) δ 177.5, 173.2, 170.52, 102.4, 94.7, 82.7, 80.1, 77.7, 77.5, 76.7, 75.3, 73.9, 73.2, 72.9, 70.3, 60.0, 67.2, 65.1, 64.3, 61.8, 51.6, 49.2, 45.2, 42.2, 41.9, 41.1, 36.8, 36.1, 35.0, 31.6, 30.3, 29.9, 29.2, 27.8, 26.4, 22.5, 21.8, 21.4, 21.3, 18.9, 18.0, 15.2, 11.3, 8.7, 7.2.

### Example 14

### 2′-O-(3-{[5-(Methyloxy)-5-oxopentanoyl]amino}propyl)-9-deoxo-9a-methyl-9a-aza-9a-homoerythromycin A

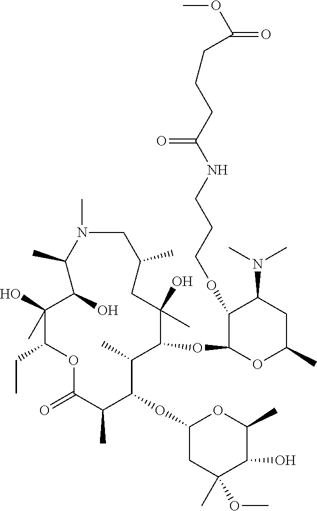

To the mono-methyl glutarate (113 mg, 0.775 mmol) diluted in DCM (5 mL) under N₂ atmosphere, triethylamine (0.5 mL), HOBt (109 mg, 0.806 mmol) and solution of EDC (202 mg, 1.054 mmol) in DCM (6 mL) were added and reaction mixture was stirred at room temperature for 15 minutes. Then solution of 2′-O-(3-aminopropyl)-9-deoxo-9a-methyl-9a-aza-9a-homoerythromycin A, Intermediate 1 (500 mg, 0.620 mmol) in DCM (9 mL) was added and stirring continued for 20 hours. Reaction mixture was extracted with saturated aqueous NaHCO₃ (2×15 mL). Combined aqueous layers were extracted with DCM (20 mL). Combined organic layers were evaporated in vacuum. White foamy solid was diluted in EtOAc (20 mL) and extracted with water (20 mL) at pH 4. Organic layer at pH 4 was discarded and aqueous extracted with DCM (3×20 mL) at pH 6. To the combined organic layers at pH 6 water (20 mL) was added, pH adjusted to 9 and layers separated. DCM was evaporated in vacuum and dried at 50° C. for 5 hours yielding title compound (435 mg) as a white foamy solid.

MS (ES+) m/z: 935.24 [MH]+.

¹³C (DMSO, 125 MHz) δ 177.5, 173.4, 171.3, 102.4, 94.7, 82.7, 80.1, 77.7, 77.5, 76.7, 75.3, 73.9, 73.2, 72.9, 70.4, 60.0, 67.2, 65.1, 64.3, 61.8, 51.6, 49.2, 45.2, 42.2, 41.9, 41.1, 36.7, 36.1, 35.0, 34.8, 33.0, 31.6, 30.1, 27.8, 26.4, 22.5, 21.8, 21.4, 21.3, 21.0, 18.9, 18.0, 15.2, 11.3, 8.7, 7.2.

### Example 15

### 2′-O-{3-[(Cyclobutylcarbonyl)amino]propyl}-9-deoxo-9a-methyl-9a-aza-9a-homoerythromycin A

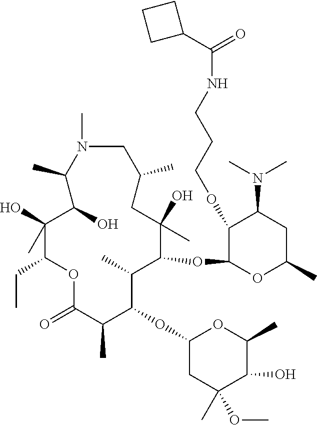

PS-Carbodiimide resin (PS-CDI, loading: 1.24 mmol/g; 390 mg, 0.806 mmol, 1.3 equivalent) was added to a dry reaction vessel. The cyclobutane carboxylic acid (39.1 mg, 0.391 mmol) and HOBt (35.2 mg, 0.261 mmol) suspended in a dry mixture of DCM (5 mL) and DMF (0.2 mL) were added to the dry resin. The mixture was stirred at room temperature for 5 minutes upon which 2′-O-(3-aminopropyl)-9-deoxo-9a-methyl-9a-aza-9a-homoerythromycin A, Intermediate 1 (300 mg, 0.372 mmol), dissolved in dry DCM (7 mL) was added. The reaction mixture was heated by microwave at 75° C. for 7 minutes. HOBt was scavenged using PS-trisannine (loading: 4.11 mmol/g) (318 mg, 5 equivalents according to HOBt) for 4 hours at room temperature. The resin was removed from the reaction mixture by filtration and washed with DCM (2×10 mL). Solvent was evaporated in vacuum yielding white foamy solid to which MeCN (2-3 mL) was added and product precipitated, filtered off yielding title compound (227 mg) as a white powder.

MS (ES+) m/z: 888.47 [MH]+.

¹³C (DMSO, 125 MHz) δ 177.5, 173.8, 102.4, 94.7, 82.5, 80.2, 77.7, 77.5, 76.7, 75.0, 73.9, 73.2, 72.9, 70.4, 68.7, 67.2, 65.2, 64.2, 62.0, 49.2, 45.2, 42.1, 41.9, 41.2, 38.2, 36.6, 36.1, 35.2, 32.2, 30.1, 27.8, 26.3, 25.1, 22.5, 21.8, 21.4, 21.3, 18.9, 18.2, 18.0, 15.2, 11.3, 8.7, 7.2.

### Example 16

### 2′-O-{3-[(Methoxyacetyl)amino]propyl}-9-deoxo-9a-methyl-9a-aza-9a-homoerythromycin A

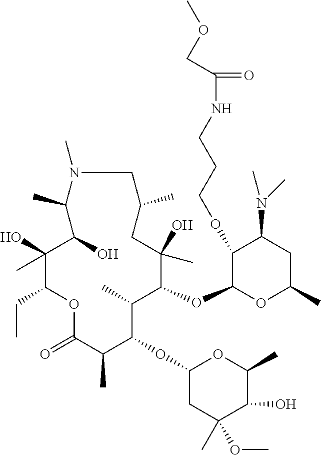

### Method A:

PS-Carbodiimide resin (PS-CDI, loading: 1.24 mmol/g; 390 mg, 0.806 mmol, 1.3 equivalents) was added to a dry reaction vessel. The methoxyacetic acid (0.030 mL, 0.391 mmol) and HOBt (35.2 mg, 0.261 mmol) suspended in a mixture of dry DCM (5 mL) and DMF (0.2 mL) were added to the dry resin. The mixture was stirred at room temperature for 5 minutes upon which 2′-O-(3-aminopropyl)-9-deoxo-9a-methyl-9a-aza-9a-homoerythromycin A, Intermediate 1 (300 mg, 0.372 mmol), dissolved in dry DCM (7 mL) was added. The reaction mixture was heated by microwave at 75° C. for 7 minutes. HOBt was scavenged using PS-trisannine (loading: 4.11 mmol/g) (318 mg, 5 equivalents according to HOBt) for 4 hours at room temperature. The resin was removed from the reaction mixture by filtration and washed with DCM (2×10 mL). Solvent was evaporated in vacuum yielding 390 mg of transparent oil that was diluted in EtOAc (20 mL) and extracted with water (20 mL) at pH 4. Organic layer was discarded and aqueous extracted with DCM (20 mL) at pH 5.8. Organic layer at pH 5.8 was discarded and aqueous extracted with DCM (3×20 mL) at pH 6.2. To the combined organic layers at pH 6.2 water was added (20 mL), pH adjusted to 9, layers separated and DCM evaporated in vacuum yielding title compound (197 mg) as a white powder.

MS (ES+) m/z: 878.42 [MH]+.

¹³C (DMSO, 125 MHz) δ 177.5, 168.8, 102.3, 94.8, 82.7, 80.0, 77.7, 77.5, 76.7, 75.3, 73.9, 73.2, 72.9, 72.0, 70.2, 69.0, 67.2, 65.1, 64.4, 61.8, 58.8, 49.2, 45.2, 42.2, 41.9, 41.1, 36.2, 36.1, 35.0, 31.6, 30.2, 27.8, 26.4, 22.5, 21.8, 21.4, 21.3, 18.9, 18.0, 15.2, 11.3, 8.7, 7.2.

### Method B:

Methoxyacetic acid (0.119 mL, 1.551 mmol) was diluted in DCM (10 ml) under N₂ atmosphere. TEA (1 mL), HOBT (0.218 g, 1.613 mmol) and solution of EDC (0.404 g, 2.109 mmol) in DCM (10 ml) were added and reaction mixture was stirred at room temperature for 15 minutes. Solution of 2′-O-(3-aminopropyl)-9-deoxo-9a-methyl-9a-aza-9a-homoerythromycin A, Intermediate 1, (1 g, 1.241 mmol) in DCM (20 ml) was added, stirring was continued for 20 hours and saturated aqueous NaHCO₃ (30 ml) was added. Layers were separated and DCM was evaporated in vacuum. Foamy solid was diluted in EtOAc (30 ml) and extracted with water (30 ml) at pH 4. Organic layer was discarded and the aqueous one washed with DCM (3×25 ml) at pH 5.8. Combined organic layers were discarded and aqueous extracted with DCM (3×30 ml) at pH 6.2. To the combined organic layers water (30 ml) was added, pH adjusted to 9 and layers separated. DCM layer was evaporated in vacuum yielding 510 mg of crude product as white foamy solid which was precipitated from diethyl-ether/n-hexane, filtered off and dried at 50° C. for 5 hours yielding title product (505.1 mg) as a white powder.

Title product obtained by Method B has identical mass and NMR data as product obtained by Method A.

### Example 17

### 2′-O-{3-[(3-Furanylcarbonyl)amino]propyl}-9-deoxo-9a-methyl-9a-aza-9a-homoerythromycin A

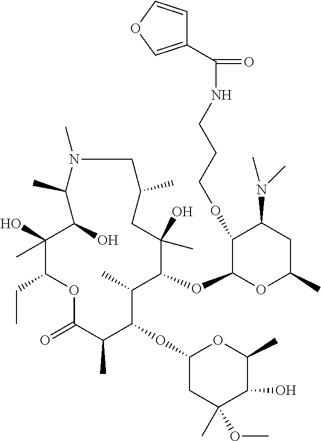

PS-Carbodiimide resin (PS-CDI, loading: 1.24 mmol/g; 390 mg, 0.806 mmol, 1.3 equivalents) was added to a dry reaction vessel. The 3-furoic acid (43.8 mg, 0.391 mmol) and HOBt (35.2 mg, 0.261 mmol) suspended in a dry mixture of DCM (5 mL) and DMF (0.2 mL) were added to the dry resin. The mixture was stirred at room temperature for 5 minutes upon which 2′-O-(3-aminopropyl)-9-deoxo-9a-methyl-9a-aza-9a-homoerythromycin A, Intermediate 1 (300 mg, 0.372 mmol), dissolved in dry DCM (7 mL) was added. The reaction mixture was heated by microwave at 75° C. for 7 minutes. HOBt was scavenged using PS-trisamine (loading: 4.11 mmol/g) (318 mg, 5 equivalents according to HOBt) for 4 hours at room temperature. The resin was removed from the reaction mixture by filtration and washed with DCM (2×10 mL). Solvent was evaporated in vacuum yielding transparent oil that was diluted in EtOAc (20 mL) and extracted with water (20 mL) at pH 4. Organic layer was discarded and aqueous extracted with DCM (20 mL) at pH 5.5. Organic layer at pH 5.5 was discarded and aqueous extracted with DCM (2×20 mL) at pH 6.3. To the combined organic layers at pH 6.3 water (20 mL) was added and pH adjusted to 9 and layers separated. DCM was evaporated in vacuum yielding white foamy solid, to which diisopropyl-ether (˜4 ml) was added. Precipitate was filtered off and dried at 50° C. for 5 hours yielding title compound (193 mg) as a white powder.

MS (ES+) m/z: 900.48 [MH]+.

¹³C (DMSO, 125 MHz) δ 177.5, 161.8, 145.0, 144.2, 123.4, 109.3, 102.4, 94.7, 82.7, 80.3, 77.7, 77.5, 76.7, 75.3, 73.9, 73.2, 72.9, 70.1, 69.0, 67.1, 65.1, 64.2, 61.8, 49.2, 45.2, 42.1, 41.9, 41.3, 36.6, 36.1, 35.0, 32.7, 30.4, 27.8, 26.3, 22.4, 21.8, 21.4, 21.3, 18.9, 18.0, 15.2, 11.3, 8.8, 7.1

### Example 18

### 2′-O-(3-{[(5-Methyl-2-pyrazinyl)carbonyl]amino}propyl)-9-deoxo-9a-methyl-9a-aza-9a-homoerythromycin A

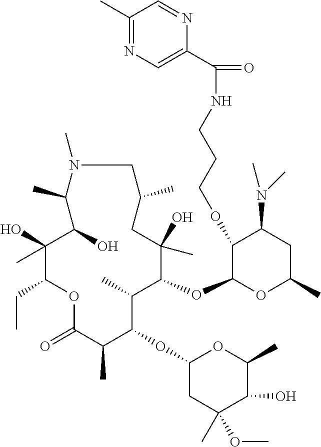

PS-Carbodiimide resin (PS-CDI, loading: 1.24 mmol/g; 390 mg, 0.806 mmol, 1.3 equivalents) was added to a dry reaction vessel. The 5-methyl-2-pyrazinecarboxylic acid (54.0 mg, 0.391 mmol) and HOBt (35.2 mg, 0.261 mmol) diluted in a dry mixture of DCM (5 mL) and DMF (0.2 mL) were added to the dry resin. The mixture was stirred at room temperature for 5 minutes upon which 2′-O-(3-aminopropyl)-9-deoxo-9a-methyl-9a-aza-9a-homoerythromycin A, Intermediate 1 (300 mg, 0.372 mmol), dissolved in dry DCM (7 mL) was added. The reaction mixture was heated by microwave at 75° C. for 7 minutes. HOBt was scavenged using PS-trisamine (loading: 4.11 mmol/g) (318 mg, 5 equivalents according to HOBt) for 4 hours at room temperature. The resin was removed from the reaction mixture by filtration and washed with DCM (2×10 mL). Solvent was evaporated in vacuum yielding 400 mg of yellowish oil that was diluted in EtOAc (20 mL) and extracted with water (20 mL) at pH 4. Organic layer at pH 4 was discarded and aqueous one extracted with DCM (20 ml) at pH 5.5. Organic layer at pH 5.5 was discarded and aqueous extracted with DCM (3×20 mL) at pH 6.2. To the combined organic layers at pH 6.2 water (20 ml) was added, pH adjusted to 9 and layers separated. DCM was evaporated in vacuum yielding white foamy solid that was precipitated from diisopropyl-ether and dried at 50° C. for 5 hours yielding title compound (198 mg) as a white powder.

MS (ES+) m/z: 926.45 [MH]+.

¹³C (DMSO, 125 MHz) δ 177.4, 163.0, 157.0, 143.1, 142.7, 142.6, 102.2, 94.8, 82.8, 79.9, 77.7, 77.5, 76.7, 75.3, 73.9, 73.1, 72.9, 70.3, 69.0, 67.1, 65.1, 64.4, 61.8, 49.1, 45.2, 42.1, 41.9, 41.1, 37.0, 36.1, 34.9, 31.4, 30.0, 27.8, 26.3, 22.4, 21.8, 21.6, 21.3, 18.9, 18.1, 15.2, 11.3, 8.7, 7.1.

### Example 19

### 2′-O-(3-{[(1,3,5-Trimethyl-1H-pyrazol-4-yl)carbonyl]amino}propyl)-9-deoxo-9a-methyl-9a-aza-9a-homoerythromycin A

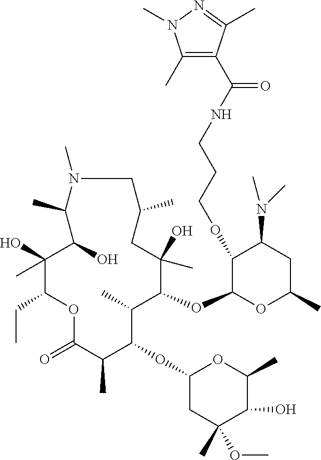

PS-Carbodiimide resin (PS-CDI, loading: 1.24 mmol/g; 390 mg, 0.806 mmol, 1.3 eq) was added to a dry reaction vessel. The 1,3,5-trimethyl-1H-pyrazole-4-carboxylic acid (60.2 mg, 0.391 mmol) and HOBt (35.2 mg, 0.261 mmol) diluted in a mixture of dry DCM (5 mL) and dry DMF (0.2 mL) were added to the dry resin. The mixture was stirred at room temperature for 5 minutes upon which 2′-O-(3-aminopropyl)-9-deoxo-9a-methyl-9a-aza-9a-homoerythromycin A, Intermediate 1 (300 mg, 0.372 mmol), dissolved in DCM (dried over molecular sieves) (7 mL) was added. The reaction mixture was heated by microwave at 75° C. for 7 minutes. HOBt was scavenged using PS-trisannine (loading: 4.11 mmol/g) (318 mg, 5 eq according to HOBt) for 4 hours at room temperature. The resin was removed from the reaction mixture by filtration and washed with DCM (2×10 mL).

Solvent was evaporated in vacuum yielding 540 mg of transparent oil that was diluted in EtOAc (20 ml) and extracted with water (20 ml) at pH 4. Organic layer was discarded and aqueous extracted with DCM (20 ml) at pH 5.5. Organic layer was discarded and aqueous extracted with DCM (3×20 ml) at pH 6. To the combined organic layers water (20 ml) was added, pH adjusted to 9 and layers separated. DCM was evaporated in vacuum yielding transparent oil which was precipitated from EtOAc/n-hexane, filtered off and dried at 50° C. for 5 hours yielding title compound (141 mg) as a white powder.

MS (ES+): 942.58 [MH]⁺

13C NMR (DMSO, 125 MHz) δ 177.5, 164.4, 144.9, 139.7, 114.9, 102.5, 94.7, 82.4, 80.6, 77.7, 77.4, 76.7, 75.3, 73.9, 73.2, 72.9, 71.3, 68.9, 67.2, 65.0, 64.0, 61.8, 49.2, 45.3, 42.2, 41.9, 40.9, 37.2, 36.1, 35.8, 35.0, 32.1, 30.5, 27.8, 26.3, 22.3, 21.8, 21.4, 21.3, 18.9, 18.0, 15.2, 13.3, 11.3, 10.5, 8.6, 7.1.

### Example 20

### 2′-O-{3-[(3-Pyridinylcarbonyl)amino]propyl}-9-deoxo-9a-methyl-9a-aza-9a-homoerythromycin A

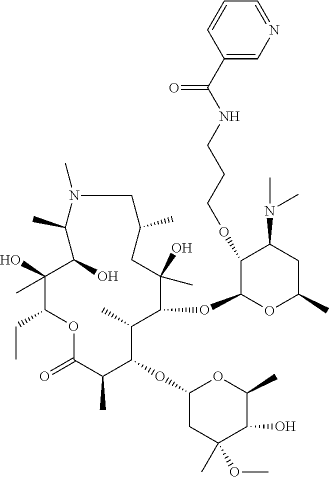

PS-Carbodiimide resin (390 mg, 0.484 mmol) was added to a reaction vessel. The nicotinic acid (48.1 mg, 0.391 mmol) and HOBt (35.2 mg, 0.261 mmol), dissolved in a mixture of dry DCM (6 mL) and dry DMF (0.3 mL), was added to the resin. The mixture was stirred at room temperature for 5 minutes upon which 2′-O-(3-aminopropyl)-9-deoxo-9a-methyl-9a-aza-9a-homoerythromycin A, Intermediate 1 (300 mg, 0.372 mmol), dissolved in dry DCM (6 mL) was added. The reaction mixture was heated by microwave at 75° C. for 7 minutes.

HOBt was scavenged using PS-trisannine (loading: 4.11 mmol/g) (318 mg, 1.305 mmol, 5 eq according to HOBt) for 3 hours at room temperature. Resin was removed by filtration, washed with DCM (2×1 mL). The organic solvent was evaporated and precipitated from diethyl ether yielding 110 mg of title compound as a white foamy product.

MS (ES+) m/z: 912.24 [MH]+

¹³C (DMSO, 125 MHz) δ 177.5, 165.0, 152.0, 148.5, 135.2, 130.8, 123.8, 102.4, 94.7, 82.7, 80.3, 77.7, 77.4, 76.7, 75.2, 74.0, 73.2, 72.9, 70.7, 68.8, 67.2, 65.1, 64.2, 61.9, 49.2, 45.2, 41.9, 41.1, 37.5, 36.1, 35.0, 32.1, 30.2, 27.8, 26.3, 22.4, 21.8, 21.4, 21.3, 18.9, 18.0, 15.2, 11.3, 8.7, 7.1

### Example 21

### 2′-O-{3-[(Ethyloxyacetyl)amino]propyl}-9-deoxo-9a-methyl-9a-aza-9a-homoerythromycin A

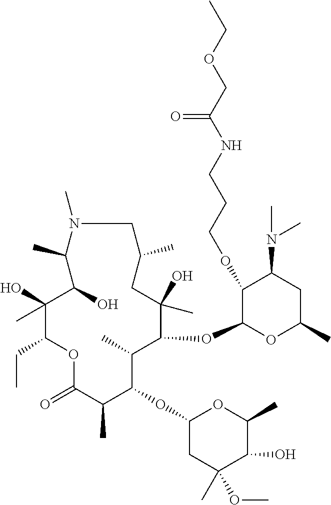

### Method A:

PS-Carbodiimide resin (390 mg, 0.484 mmol) was added to a reaction vessel. The (ethyloxy)acetic acid (40.7 mg, 0.391 mmol) and HOBt (35.2 mg, 0.261 mmol), dissolved in a mixture of dry DCM (6 mL) and dry DMF (0.2 mL), was added to the dry resin. The mixture was stirred at room temperature for 5 minutes upon which 2′-O-(3-aminopropyl)-9-deoxo-9a-methyl-9a-aza-9a-homoerythromycin A, Intermediate 1, (300 mg, 0.372 mmol), dissolved in dry DCM (6 mL) was added. The reaction mixture was heated by microwave at 75° C. for 7 minutes.

HOBt was scavenged using PS-trisamine (loading: 4.11 mmol/g) (318 mg, 1.305 mmol, 5 eq according to HOBt) for 3 hours at room temperature. Resin was removed by filtration, washed with DCM (2×1 mL). The organic solvent was evaporated.

The foamy white solid was dissolved in EtOAc (20 ml) and H₂O (20 ml) was added and pH was adjusted to pH 3.7. Layers were separated, to the H₂O layer DCM (10 ml) was added and pH was adjusted to pH 5.5. Some of the impurities were separated to DCM layer while product was left in H₂O layer. This extraction was repeated for 2 times.

To the H₂O layer at pH 5.5 DCM (10 ml) was added and pH was adjusted to pH 6.1.

Layers were separated and extraction with DCM was repeated for 4 times. To the combined DCM layers H₂O (20 ml) was added and pH adjusted to pH 9.5. Layers were separated and DCM layer was evaporated in vacuum yielding 190 mg of title compound.

MS (ES+) m/z: 892.52 [MH]+

¹³C (DMSO, 125 MHz) δ 177.5, 169.2, 102.3, 94.7, 82.7, 80.1, 77.7, 77.5, 76.7, 75.3, 74.0, 73.2, 70.1, 69.1, 67.2, 66.4, 65.1, 64.4, 61.8, 49.2, 45.2, 42.2, 41.9, 41.1, 36.1, 35.0, 31.8, 30.3, 27.8, 26.4, 22.5, 21.8, 21.4, 21.3, 18.9, 18.0, 15.2, 15.1, 11.3, 8.7, 7.2

### Method B:

To the solution of (ethyloxy)acetic acid (1.468 mL, 15.51 mmol) in DCM (200 ml), triethylamine (10 mL), HOBT (2.470 g, 16.13 mmol) and solution of EDC (4.04 g, 21.09 mmol) in DCM (100 ml) were added and reaction mixture was stirred under N₂ for 15 minutes. Solution of the 2′-O-(3-aminopropyl)-9-deoxo-9a-methyl-9a-aza-9a-homoerythromycin A, Intermediate 1, (10 g, 12.41 mmol) in DCM (100 ml) was added, reaction mixture stirred at room temperature under N₂ over night and saturated aquous NaHCO₃ (400 ml) was added. Layers were separated and aqueous layer was extracted with DCM (1×150 ml). Combined DCM layers were evaporated in vacuum.

The foamy white solid was dissolved in EtOAc (60 ml), H₂O (40 ml) was added and pH was adjusted to 3.7. Layers were separated, to the H₂O layer DCM was added (30 ml) and pH adjusted to 5.5. This extraction was repeated for 2 times.

To the H₂O layer at pH 5.5 DCM (10 ml) was added and pH was adjusted to 5.8. Layers were separated aqueous one extracted with DCM (4×). To the combined DCM layers H₂O (20 ml) was added and pH was adjusted to 9.5. Layers were separated, DCM layer was evaporated in vacuum, residue precipitated from acetone (3 ml)/H₂O (50 ml) and dried at 70° C. for 6 hours yielding 1.7 g of title product.

Additional amount of product was obtained by extraction H₂O layer at pH 5.8 with DCM (repeated 6 times). To the combined DCM layers H₂O (20 ml) was added, pH was adjusted to 9.5 and layers separated, DCM evaporated in vacuum, residue precipitated from acetone (5 ml)/H₂O (60 ml) and dried at 70° C. for 6 hours yielding additional 2.7 g of title product.

### General Procedure for Examples 22 to 23

The corresponding acid (1.25 eq.) suspended in a dry DCM (5 ml) was added to a round bottom flask. HOBt (1.3 eq.) and Et₃N (5.8 eq.) were added followed by the addition of a suspension of EDAC (1.7 eq.) in a dry DCM (6 ml). After stirring at room temperature for 15 minutes 2′-O-(3-aminopropyl)-9-deoxo-9a-methyl-9a-aza-9a-homoerythromycin A, Intermediate 1 dissolved in dry DCM (9 mL) was added. Stirring was continued at room temperature for 20 hours. Reaction mixture was extracted with saturated aqueous NaHCO₃ (2×15 ml). Organic solvent was evaporated.

Purification was performed by acid-base extraction affording corresponding compound of examples 22 to 23.

### Example 22

### 2′-O-(3-{[3-Hydroxy-2-(hydroxymethyl)-2-methylpropanoyl]amino}propyl)-9-deoxo-9a-methyl-9a-aza-9a-homoerythromycin A

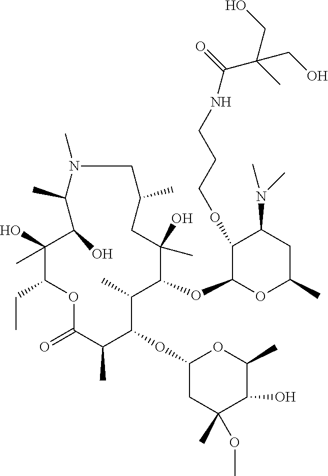

According to the general procedure for Examples 22 to 23 starting from 2′-O-(3-aminopropyl)-9-deoxo-9a-methyl-9a-aza-9a-homoerythromycin A, Intermediate 1 (500 mg, 0.620 mmol) and 2,2-Bis-(hydroxmethyl)propionic acid (1.25 equiv), crude product was obtained which was dissolved in EtOAc and extracted with water at pH 4. Organic layer at pH 4 was discarded and aqueous washed with DCM at pH 6.5. Organic layer at pH 6.5 was discarded and aqueous extracted with DCM at pH 7.4. To the combined organic layers at pH 7.4 water was added, pH adjusted to 9 and layers separated. DCM layer was evaporated in vacuum and residue precipitated from CH₃CN to yield title compound (235 mg).

MS (ES+) m/z: 923.1 [MH]+

### Example 23

### 2′-O-{3-[(2-Pyrazinylcarbonyl)amino]propyl}-9-deoxo-9a-methyl-9a-aza-9a-homoerythromycin A

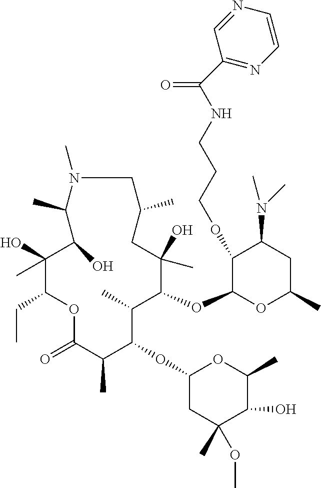

According to the general procedure for Examples 22 to 23 starting from 2′-O-(3-aminopropyl)-9-deoxo-9a-methyl-9a-aza-9a-homoerythromycin A, Intermediate 1 (500 mg, 0.620 mmol) and 2-pyrazinecarboxylic acid (1.25 equiv), crude product was obtained which was dissolved in EtOAc and extracted with water at pH 4. Organic layer at pH 4 was discarded. To the aqueous layer DCM was added, pH adjusted to 5.9 and aqueous layer extracted with DCM. To the combined organic layers at pH 5.9 water was added, pH adjusted to 9 and layers separated. DCM layer was evaporated in vacuum and residue precipitated from diisopropylether to yield title compound (236 mg).

MS (ES+) m/z: 913.27 [MH]+

### General Procedure for Examples 24 to 40

Dry PS-Carbodiimide resin (PS-CDI, loading: 1.2 mmol/g, 1.3 eq) was added to a reaction vessel. The corresponding acid (1.05 eq) and HOBt (0.7 eq), dissolved in a dry mixture of DCM (6 mL) and DMF (100-500 μL), were added to the resin. The mixture was stirred at room temperature for 5 minutes upon which 2′-O-(3-aminopropyl)-9-deoxo-9a-methyl-9a-aza-9a-homoerythromycin A, Intermediate 1 dissolved in dry DCM (6 mL) was added. The reaction mixture was heated by microwave irradiation at 70-75° C. for 7-8 minutes.

HOBt was scavenged using PS-trisannine (loading: 4.11 mmol/g, 5 equivalent according to HOBt) for 3 hours at room temperature. The resin was removed from the reaction mixture by filtration and washed with DCM (2-5 mL). Organic solvent was evaporated to yield crude product of Examples 24-40.

Purification was performed by acid-base extraction or by precipitation or by HPLC technique.

### Example 24

### 2′-O-(3-{[(2,5-Dimethyl-3-furanyl)carbonyl]amino}propyl)-9-deoxo-9a-methyl-9a-aza-9a-homoerythromycin A

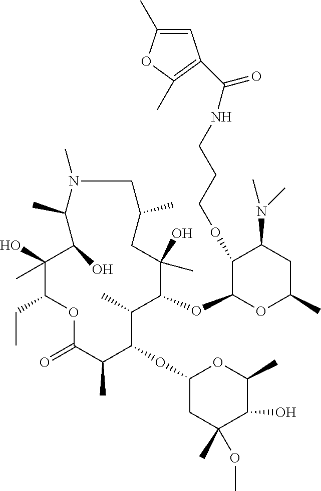

According to the general procedure for Examples 24 to 40 starting from 2′-O-(3-aminopropyl)-9-deoxo-9a-methyl-9a-aza-9a-homoerythromycin A, Intermediate 1 (300 mg, 0.372 mmol) and 2,5-dimethyl-3-furancarboxylic acid (1.05 equiv), crude product was obtained which was dissolved in EtOAc and extracted with water at pH 4. Organic layer at pH 4 was discarded and aqueous washed with DCM at pH 5.5. Organic layer at pH 5.5 was discarded and aqueous extracted with DCM at pH 6.2. To the combined organic layers at pH 6.2 water was added, pH adjusted to 9 and layers separated. DCM layer was evaporated in vacuum and residue precipitated from diisopropylether to yield title compound (193 mg).

MS (ES+) m/z: 928.48 [MH]+

### Example 25

### 2′-1-{3-[(Tetrahydro-2H-pyran-4-ylcarbonyl)amino]propyl}-9-deoxo-9a-methyl-9a-aza-9a-homoerythromycin A

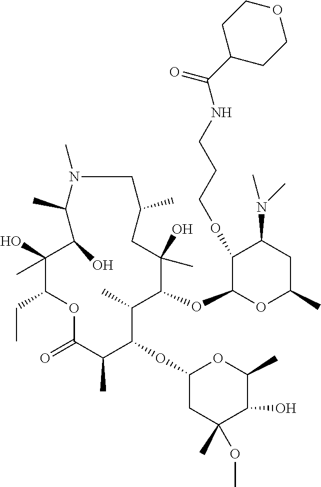

According to the general procedure for Examples 24 to 40 starting from 2′-O-(3-aminopropyl)-9-deoxo-9a-methyl-9a-aza-9a-homoerythromycin A, Intermediate 1 (300 mg, 0.372 mmol) and tetrahydro-2H-pyran-4-carboxylic acid (1.05 equiv), crude product was obtained which was dissolved in EtOAc and extracted with water at pH 4. Organic layer at pH 4 was discarded and aqueous washed with DCM at pH 5.5. Organic layer at pH 5.5 was discarded and aqueous extracted with DCM at pH 6.0. To the combined organic layers at pH 6.0 water was added, pH adjusted to 9 and layers separated. DCM layer was evaporated in vacuum and residue precipitated from EtOAc/n-hexane to yield title compound (158 mg).

MS (ES+) m/z: 918.6 [MH]+

### Example 26

### 2′-O-(3-{[(1,2,5-Trimethyl-1H-pyrrol-3-yl)carbonyl]amino}propyl)-9-deoxo-9a-methyl-9a-aza-9a-homoerythromycin A

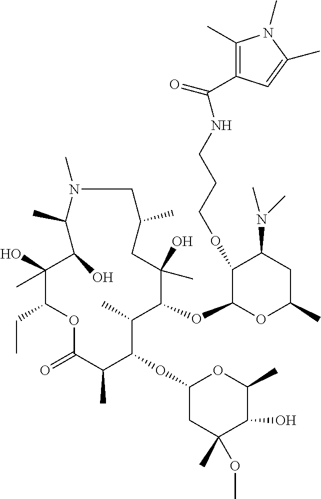

According to the general procedure for Examples 24 to 40 starting from 2′-O-(3-aminopropyl)-9-deoxo-9a-methyl-9a-aza-9a-homoerythromycin A, Intermediate 1 (300 mg, 0.372 mmol) and 1,2,5-trimethyl-1H-pyrrole-3-carboxylic acid (1.05 equiv), crude product was obtained which was dissolved in EtOAc and extracted with water at pH 4. Organic layer at pH 4 was discarded. To the aqueous layer DCM was added, pH adjusted to 5.8 and aqueous layer extracted with DCM. To the combined organic layers at pH 5.8 water was added, pH adjusted to 9 and layers separated. DCM layer was evaporated in vacuum and residue precipitated from EtOAc/n-hexane to yield title compound (155 mg).

MS (ES+) m/z: 941.7 [MH]+

### Example 27

### 2′-O-(3-{[(1-Methyl-1H-imidazol-5-yl)carbonyl]amino}propyl)-9-deoxo-9a-methyl-9a-aza-9a-homoerythromycin A

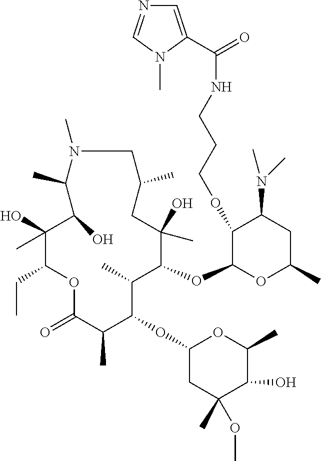

According to the general procedure for Examples 24 to 40 starting from 2′-O-(3-aminopropyl)-9-deoxo-9a-methyl-9a-aza-9a-homoerythromycin A, Intermediate 1 (300 mg, 0.372 mmol) and 1-methyl-1H-imidazole-5-carboxylic acid (1.05 equiv), crude product was obtained which was dissolved in EtOAc and extracted with water at pH 4. Organic layer at pH 4 was discarded. To the aqueous layer DCM was added, pH adjusted to 6.5 and aqueous layer extracted with DCM. To the combined organic layers at pH 6.5 water was added, pH adjusted to 9 and layers separated. DCM layer was evaporated in vacuum and residue precipitated from EtOAc/n-hexane to yield title compound (165 mg).

MS (ES+) m/z: 914.44 [MH]+

### Example 28

2′-O-[3-({[1-Methyl-3-(trifluoromethyl)-1H-pyrazol-4-yl]carbonyl}amino)propyl]-9-deoxo-9a-methyl-9a-aza-9a-homoerythromycin A

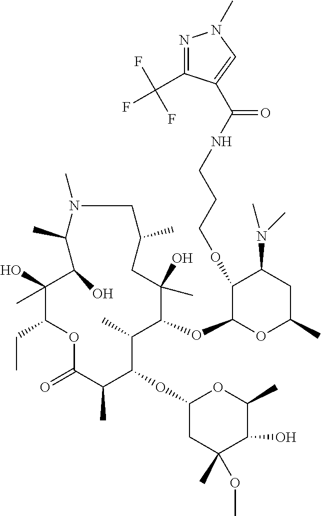

According to the general procedure for Examples 24 to 40 starting from 2′-O-(3-aminopropyl)-9-deoxo-9a-methyl-9a-aza-9a-homoerythromycin A, Intermediate 1 (300 mg, 0.372 mmol) and 1-methyl-3-(trifluoromethyl)-1H-pyrazole-4-carboxylic acid (1.05 equiv), crude product was obtained which was dissolved in EtOAc and extracted with water at pH 4. Organic layer at pH 4 was discarded. To the aqueous layer DCM was added, pH adjusted to 5.8 and aqueous layer extracted with DCM. To the combined organic layers at pH 5.8 water was added, pH adjusted to 9 and layers separated. DCM layer was evaporated in vacuum and residue precipitated from EtOAc/n-hexane to yield title compound (160 mg).

MS (ES+) m/z: 982.38 [MH]+

### Example 29

### 2′-O-(3-{[(4-Chlorophenyl)carbonyl]amino}propyl)-9-deoxo-9a-methyl-9a-aza-9a-homoerythromycin A

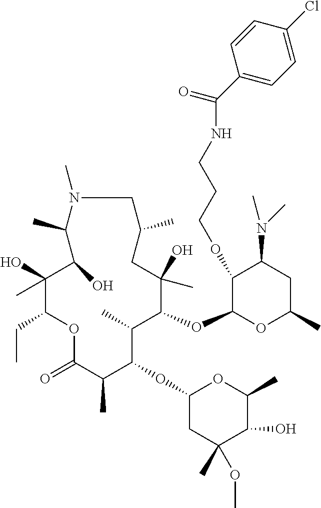

According to the general procedure for Examples 24 to 40 starting from 2′-O-(3-aminopropyl)-9-deoxo-9a-methyl-9a-aza-9a-homoerythromycin A, Intermediate 1 (300 mg, 0.372 mmol) and 4-chlorobenzoic acid (1.05 equiv), crude product was obtained which was dissolved in EtOAc and extracted with water at pH 4. Organic layer at pH 4 was discarded. To the aqueous layer DCM was added, pH adjusted to 5.4 and aqueous layer extracted with DCM. To the combined organic layers at pH 5.4 water was added, pH adjusted to 9 and layers separated. DCM layer was evaporated in vacuum and residue precipitated from diisopropylether/n-hexane to yield title compound (170 mg).

MS (ES+) m/z: 944.40 [MH]+

### Example 30

### 2′-O-{3-[(Hydroxyacetyl)amino]propyl}-9-deoxo-9a-methyl-9a-aza-9a-homoerythromycin A

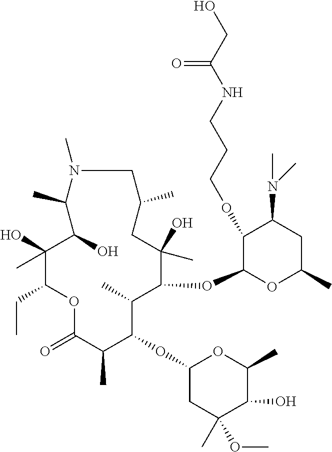

According to the general procedure for Examples 24 to 40 starting from 2′-O-(3-aminopropyl)-9-deoxo-9a-methyl-9a-aza-9a-homoerythromycin A, Intermediate 1 (300 mg, 0.372 mmol) and glicolic acid (1.05 equiv), crude product was obtained which was dissolved in EtOAc and extracted with water at pH 3.7. Organic layer at pH 3.7 was discarded and aqueous washed with DCM at pH 6.3. Organic layer at pH 6.3 was discarded and aqueous extracted with DCM at pH 7.0. To the combined organic layers at pH 7.0 water was added, pH adjusted to 9.5 and layers separated. DCM layer was evaporated in vacuum and residue precipitated from diethylether to yield title compound (302 mg).

MS (ES+) m/z: 864.50 [MH]+

### Example 31

### 2′-O-{3-[(2-Pyridinylcarbonyl)amino]propyl}-9-deoxo-9a-methyl-9a-aza-9a-homoerythromycin A

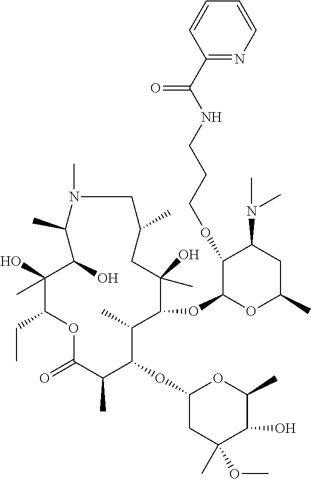

According to the general procedure for Examples 24 to 40 starting from 2′-O-(3-aminopropyl)-9-deoxo-9a-methyl-9a-aza-9a-homoerythromycin A, Intermediate 1 (300 mg, 0.372 mmol) and 2-pyridinecarboxylic acid (1.05 equiv), crude product was obtained which was precipitated from diethylether to give title product (59 mg). Mother liquor was evaporated and residue precipitated from CH₃CN to give additional amount of title product (100 mg).

MS (ES+) m/z: 911.34 [MH]+

### Example 32

### 2′-O-{3-[(2,5-Dihydroxyphenylcarbonyl)amino]propyl}-9-deoxo-9a-methyl-9a-aza-9a-homoerythromycin A

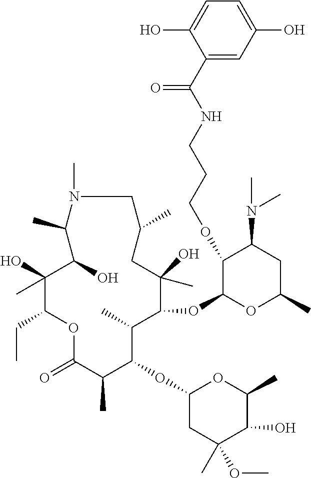

According to the general procedure for Examples 24 to 40 starting from 2′-O-(3-aminopropyl)-9-deoxo-9a-methyl-9a-aza-9a-homoerythromycin A, Intermediate 1 (300 mg, 0.372 mmol) and 2,5-dihydroxybenzoic acid (1.05 equiv), crude product was obtained which was dissolved in EtOAc and extracted with water at pH 3.7. Organic layer at pH 3.7 was discarded and aqueous washed with DCM at pH 5.5. Organic layer at pH 5.5 was discarded and aqueous extracted with DCM at pH 6.3. To the combined organic layers at pH 6.3 water was added, pH adjusted to 9.5 and layers separated. DCM layer was evaporated in vacuum and residue precipitated from CH₃CN to yield title compound (17 mg). Mother liquor was evaporated and residue precipitated from diethylether to give additional amount of title product (32 mg).

MS (ES+) m/z: 942.44 [MH]+

### Example 33

### 2′-O-(3-{[(4-Amino-2-hydroxyphenyl)carbonyl]amino}propyl)-9-deoxo-9a-methyl-9a-aza-9a-homoerythromycin A

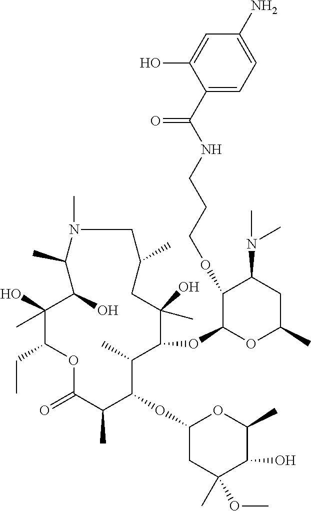

According to the general procedure for Examples 24 to 40 starting from 2′-O-(3-aminopropyl)-9-deoxo-9a-methyl-9a-aza-9a-homoerythromycin A, Intermediate 1 (300 mg, 0.372 mmol) and 4-amino-2-hydroxybenzoic acid (1.05 equiv), crude product was obtained which was purified by HPLC using mixture of eluents A (10 mM NH₄HCO₃/pH 10) and B (CH₃CN) applying gradient technique.—Title compound (150.4 mg) was obtained.

MS (ES+) m/z: 941.60 [MH]+

### Example 34

### 2′-O-(3-{[(2-Chloro-3-pyridinyl)carbonyl]amino}propyl)-9-deoxo-9a-methyl-9a-aza-9a-homoerythromycin A

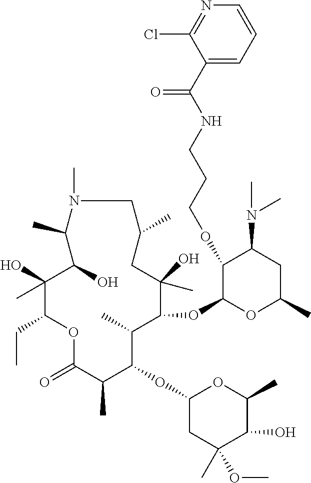

According to the general procedure for Examples 24 to 40 starting from 2′-O-(3-aminopropyl)-9-deoxo-9a-methyl-9a-aza-9a-homoerythromycin A, Intermediate 1 (300 mg, 0.372 mmol) and 2-chloro-3-pyridinecarboxylic acid (1.05 equiv), crude product was obtained which was dissolved in EtOAc and extracted with water at pH 3.7. Organic layer at pH 3.7 was discarded. To the aqueous layer DCM was added and pH adjusted to 6.0 and aqueous layer extracted with DCM. To the combined organic layers at pH 6.0 water was added, pH adjusted to 9.5 and layers separated. DCM layer was evaporated in vacuum and residue precipitated from diisopropylether to yield title compound (220 mg).

MS (ES+) m/z: 945.34 [MH]+

### Example 35

### 2′-O-(3-{[(2-Chloro-6-methyl-3-pyridinyl)carbonyl]amino}propyl)-9-deoxo-9a-methyl-9a-aza-9a-homoerythromycin A

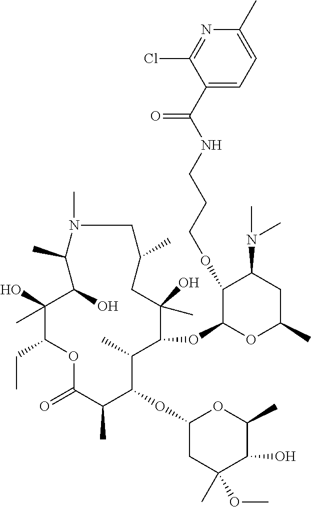

According to the general procedure for Examples 24 to 40 starting from 2′-O-(3-aminopropyl)-9-deoxo-9a-methyl-9a-aza-9a-homoerythromycin A, Intermediate 1 (300 mg, 0.372 mmol) and 2-chloro-6-methyl-3-pyridinecarboxylic acid (1.05 equiv), crude product was obtained which was dissolved in EtOAc and extracted with water at pH 3.7. Organic layer at pH 3.7 was discarded and aqueous washed with DCM at pH 5.8. Organic layer at pH 5.8 was discarded and aqueous extracted with DCM at pH 6.0. To the combined organic layers at pH 6.0 water was added, pH adjusted to 9.5 and layers separated. DCM layer was evaporated in vacuum and residue precipitated from diisopropylether to yield title compound (218 mg).

MS (ES+) m/z: 959.44 [MH]+.

### Example 36

### 2′-O-(3-{[(5-Amino-3-pyridinyl)carbonyl]amino}propyl)-9-deoxo-9a-methyl-9a-aza-9a-homoerythromycin A

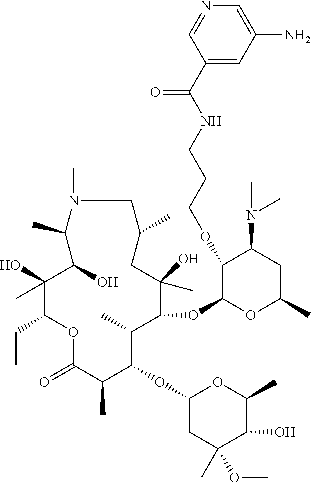

According to the general procedure for Examples 24 to 40 starting from 2′-O-(3-aminopropyl)-9-deoxo-9a-methyl-9a-aza-9a-homoerythromycin A, Intermediate 1 (300 mg, 0.372 mmol) and 5-aminonicotinic acid (1.05 equiv), crude product was obtained which was dissolved in EtOAc and extracted with water at pH 3.7. Organic layer at pH 3.7 was discarded. To the aqueous layer DCM was added and pH adjusted to 6.5 and aqueous layer extracted with DCM. To the combined organic layers at pH 6.5 water was added, pH adjusted to 9.5 and layers separated. DCM layer was evaporated in vacuum and residue precipitated from CH₃CN/diisopropylether to yield title compound (95 mg).

MS (ES+) m/z: 926.63 [MH]+

### Example 37

### 2′-O-(3-{[(3-Amino-2-pyrazinyl)carbonyl]amino}propyl)-9-deoxo-9a-methyl-9a-aza-9a-homoerythromycin A

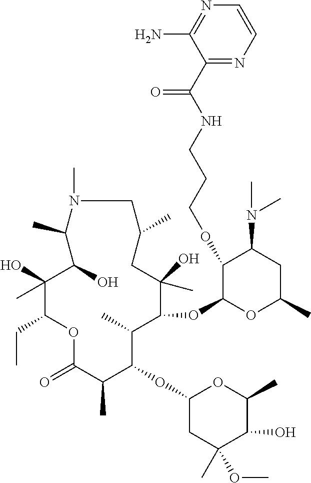

According to the general procedure for Examples 24 to 40 starting from 2′-O-(3-aminopropyl)-9-deoxo-9a-methyl-9a-aza-9a-homoerythromycin A, Intermediate 1 (300 mg, 0.372 mmol) and 3-amino-2-pyrazinecarboxylic acid (1.05 equiv), crude product was obtained which was dissolved in EtOAc and extracted with water at pH 3.7. Organic layer at pH 3.7 was discarded. To the aqueous layer DCM was added, pH adjusted to 5.9 and aqueous layer extracted with DCM. To the combined organic layers at pH 5.9 water was added, pH adjusted to 9.5 and layers separated. DCM layer was evaporated in vacuum and residue precipitated from diethylether to yield title compound (70 mg).

MS (ES+) m/z: 927.58 [MH]+

### Example 38

### 2′-O-(3-{[(5-Chloro-1,3-dimethyl-1H-pyrazol-4-yl)carbonyl]amino}propyl)-9-deoxo-9a-methyl-9a-aza-9a-homoerythromycin A

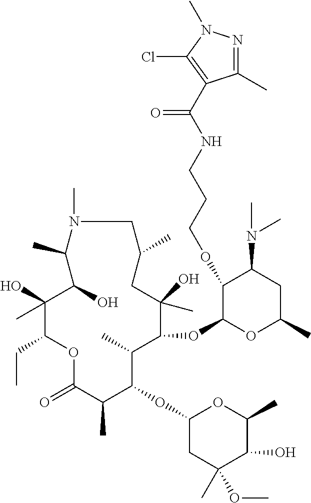

According to the general procedure for Examples 24 to 40 starting from 2′-O-(3-aminopropyl)-9-deoxo-9a-methyl-9a-aza-9a-homoerythromycin A, Intermediate 1 (300 mg, 0.372 mmol) and 5-chloro-1,3-dimethyl-1H-pyrazole-4-carboxylic acid (1.05 equiv), crude product was obtained which was dissolved in EtOAc and extracted with water at pH 3.7. Organic layer at pH 3.7 was discarded. To the aqueous layer DCM was added, pH adjusted to 6.0 and aqueous layer extracted with DCM. To the combined organic layers at pH 6.0 water was added, pH adjusted to 9.5 and layers separated. DCM layer was evaporated in vacuum and residue precipitated from diethylether/n-hexane to yield title compound (166 mg).

MS (ES+) m/z: 962.60 [MH]+

### Example 39

### 2′-O-(3-{[(2,5-Dimethyl-1,3-oxazol-4-yl)carbonyl]amino}propyl)-9-deoxo-9a-methyl-9a-aza-9a-homoerythromycin A

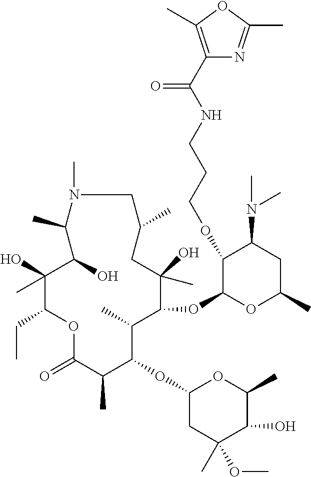

According to the general procedure for Examples 24 to 40 starting from 2′-O-(3-aminopropyl)-9-deoxo-9a-methyl-9a-aza-9a-homoerythromycin A, Intermediate 1 (300 mg, 0.372 mmol) and 2,5-dimethyl-1,3-oxazole-4-carboxylic acid (1.05 equiv), crude product was obtained which was dissolved in EtOAc and extracted with water at pH 3.7. Organic layer at pH 3.7 was discarded. To the aqueous layer DCM was added and pH adjusted to 5.8 and aqueous layer extracted with DCM. To the combined organic layers at pH 5.8 water was added, pH adjusted to 9.5 and layers separated. DCM layer was evaporated in vacuum to yield title compound (202 mg).

MS (ES+) m/z: 929.42 [MH]+

### Example 40

### 2′-O-{3-[(1-Methyl-L-prolyl)amino]propyl}-9-deoxo-9a-methyl-9a-aza-9a-homoerythromycin A

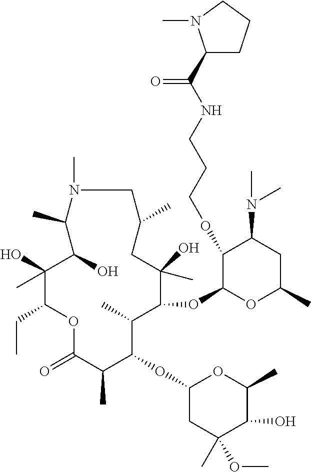

According to the general procedure for Examples 24 to 40 starting from 2′-O-(3-aminopropyl)-9-deoxo-9a-methyl-9a-aza-9a-homoerythromycin A, Intermediate 1 (300 mg, 0.372 mmol) and N-methyl-L-proline (1.05 equiv), crude product was obtained which was dissolved in EtOAc and extracted with water at pH 3.7. Organic layer at pH 3.7 was discarded and aqueous washed with DCM at pH 5.8. Organic layer at pH 5.8 was discarded and aqueous extracted with DCM at pH 6.2. To the combined organic layers at pH 6.2 water was added, pH adjusted to 9.5 and layers separated. DCM layer was evaporated in vacuum to yield title compound (147.6 mg).

MS (ES+) m/z: 917.84 [MH]+

### Intermediate 2

### 2′-O-{3-[(3-Methyl-2-butenoyl)amino]propyl}-9-deoxo-9a-methyl-9a-aza-9a-homoerythromycin A

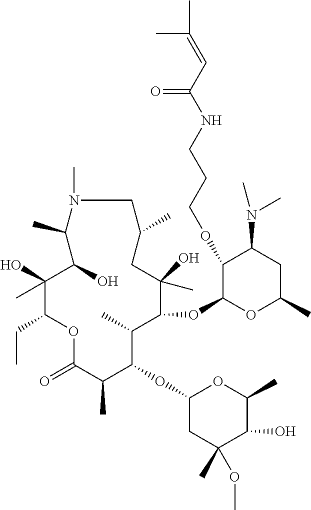

According to the general procedure for Examples 22 to 23 starting from 2′-O-(3-aminopropyl)-9-deoxo-9a-methyl-9a-aza-9a-homoerythromycin A, Intermediate 1, (500 mg, 0.620 mmol) and 3,3-dimethylacrylic acid (1.25 equiv), title compound (355 mg) was obtained.

MS (ES+) m/z: 889.24 [MH]+

### Example 41

### 2′-O-{3-[(3-Methylbutanoyl)amino]propyl}-9-deoxo-9a-methyl-9a-aza-9a-homoerythromycin A

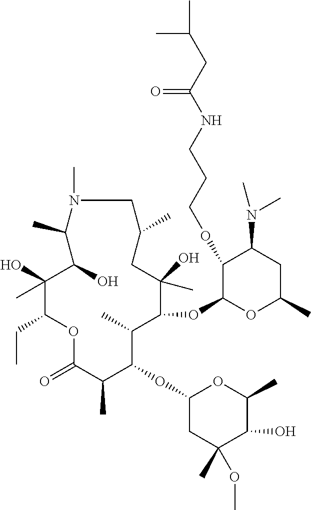

2′-O-{3-[(3-Methyl-2-butenoyl)amino]propyl}-9-deoxo-9a-methyl-9a-aza-9a-homoerythromycin A, Intermediate 2, (250 mg, 0.28 mmol) was dissolved in MeOH (25 ml). 10% Pd/C (25 mg) was added and hydrogenation performed by H₂ pressure at 2.5 bars in Parr aparatus for 17 hours. Catalyst was removed by filtration and solvent evaporated in vacuum. Foamy solid was dissolved in EtOAc (20 ml) and extracted with water (20 ml) at pH 4. Organic layer was discarded and aqueous layer extracted with DCM (20 ml) at pH 9. DCM was evaporated in vacuum yielding a crude product (215 mg) as a white foamy solid. MeCN (3 ml) was added, precipitate was filtered off and dried at 50° C. for 5 hours yielding title compound (113 mg) of as a white powder.

MS (ES+) m/z: 891.26 [MH]+

### In Vitro Assay

The in vitro potency of compounds of the invention has been measured using the methodology described in the in vitro protocol for Inhibition of IL-6 production in LPS-stimulated murine spleenocytes in vitro. Compounds of examples 8, 9, 22, 30 and 36 exhibited less than 40% inhibition of interleukin-6 (IL-6) production.

Compounds of examples 1-7, 10-21, 23-29, 31-35 and 37-41 exhibited more than 40% inhibition of interleukin-6 (IL-6) production in LPS-stimulated spleenocytes at 50 μM concentration of the compound.

### In Vivo Assay

The in vivo potency of compounds of the invention has been measured using the methodology described in the in vivo protocol for Lung neutrophilia induced by bacterial lipopolysaccharide in male BALB/cJ mice—Method A or Method B.

The compounds of examples 2, 5 and 8, showed more than 50% inhibition of total cell number and number of neutrophils in BALF of treated animals which received intraperitoneally (i.p.) a single dose of 200 mg/kg of test compound in Lung neutrophilia induced by bacterial lipopolysaccharide in male BALB/cJ mice—Method A.

Compounds of examples 13, 16-18, 19, 21 and 25 showed more than 50% inhibition and compounds of examples 7, 14, 15 and 20 showed more than 30% inhibition of total cell number and number of neutrophils or inhibition of total cell number and decrease in myeloperoxidase concentration in BALF of treated animals which received intraperitoneally (i.p.) a single dose of 100 mg/kg of test compound tested in Lung neutrophilia induced by bacterial lipopolysaccharide in male BALB/cJ mice—Method B.

Compounds of Examples 2, 16 and 21 were tested in Cigarette-smoke-induced lung neutrophilia assay. Compounds of Examples 16 and 21 statistically significantly reduced neutrophil number in BALF showing more than 40% inhibition at a dose of 30 mg/kg. The compound of Example 2 showed less than 10% inhibition of neutrophil number in BALF at a dose of 100 mg/kg with no statistical significance.

1. A compound of Formula (I): 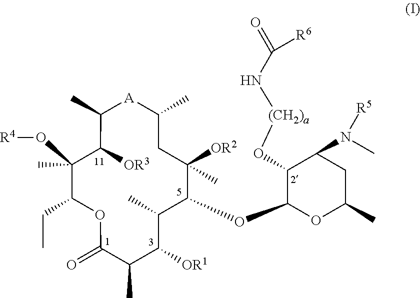
wherein:
A represents a bivalent radical —C(O)—, —N(R⁹)CH₂—, —CH₂N(R⁹)—, —CH(NR¹⁰R¹¹)—, —C(═NR¹²)—, or —CH(OH)—;
R¹ represents a α-L-cladinosyl group of Formula (a) 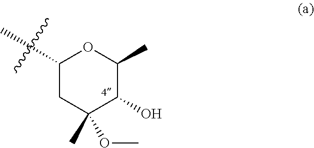
R² represents H or —CH₃;
R³ represents H or —C(O)C₁₋₃alkyl; or R³ and R⁴ taken together with the intervening atoms form a cyclic carbonate group of Formula (b): 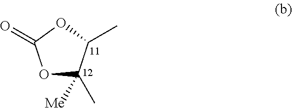
R⁴ represents H; or R³ and R⁴ taken together with the intervening atoms form a cyclic carbonate group of Formula (b);
R⁵ represents H, —C₁₋₄alkyl or —C(O)C₁₋₃alkyl;
R⁶ represents
(i) —C₁₋₈alkyl, unsubstituted or substituted at the terminal carbon atom by a group selected from hydroxy, —C₁₋₃alkoxy and —C(O)OC₁₋₃alkyl, or when
—C₁₋₈alkyl is branched, substitution can alternatively be by a hydroxyl group at each of two terminal carbon atoms,
(ii) —CH(NH₂)C₁₋₄alkyl, wherein the —C₁₋₄alkyl group may be optionally interrupted by a heteroatom selected from O, S and N,
(iii) —CH₂N(R⁷)(R⁸), wherein R⁷ and R⁸ each independently represent H or
—C₁₋₃alkyl provided that R⁷ and R⁸ cannot both simultaneously represent H,
(iv) a 4-6-membered heterocyclic ring containing up to 2 heteroatoms independently selected from O, S and N, wherein the heterocyclic ring is unsubstituted or substituted by —C₁₋₃alkyl,
(v) 5-6 membered heteroaromatic ring, unsubstituted or substituted by one to three groups independently selected from halo, hydroxyl, —C₁₋₃alkyl,
—C₁₋₃alkoxy, —CF₃, —OCF₃ and —NH₂,
(vi) —CH(NH₂)CH₂-aryl wherein the aryl group may be unsubstituted or substituted by one or two substituents independently selected from —C₁₋₃alkyl, —C₁₋₃alkoxy and hydroxyl,
(vii) —C₃₋₇cycloalkyl, or
(viii) phenyl unsubstituted or substituted by one or two groups independently selected from halo, hydroxyl, —C₁₋₃alkyl, —C₁₋₃alkoxy, —CF₃, —OCF₃ and —NH₂,
R⁹ represents H or —C₁₋₄alkyl;
R¹⁰ and R¹¹ each independently represent H, —C₁₋₆alkyl or —C(O)R⁹;
R¹² is —OR¹³;
R¹³ is H or —C₁₋₆alkyl, unsubstituted or substituted by one or two substituents independently selected from cyano, —NR¹⁴R¹⁵ and —C₁₋₆alkoxy; or —C₃₋₇cycloalkyl; or —C₃₋₆alkenyl;
R¹⁴ and R¹⁵ are independently H or —C₁₋₆alkyl;
and
a is an integer from 2 to 6;
or a salt thereof.

2. A compound as claimed in claim 1, wherein A is a bivalent radical —N(R⁹)CH₂— wherein R⁹ is —C₁₋₄alkyl.

3. A compound as claimed in claim 1, wherein R² is H.

4. A compound as claimed in claim 1, wherein R³ is H.

5. A compound as claimed in claim 1, wherein R⁴ is H.

6. A compound as claimed in claim 1, wherein R⁵ is methyl.

7. A compound as claimed in claim 1 wherein R⁶ is —C₁₋₈alkyl substituted at the terminal carbon atom by —C₁₋₃alkoxy.

8. A compound as claimed in claim 1, wherein a is 3.

9. A compound of Formula (I) as claimed in claim 1, selected from:
2′-O-[3-(Acetylamino)propyl]-9-deoxo-9a-methyl-9a-aza-9a-homoerythromycin A,
2′-O-[3-(Propanoylamino)propyl]-9-deoxo-9a-methyl-9a-aza-9a-homoerythromycin A,
2′-O-{3-[(2-Methylpropanoyl)amino]propyl}-9-deoxo-9a-methyl-9a-aza-9a-homoerythromycin A,
2′-O-{3-[(2,2-Dimethylpropanoyl)amino]propyl}-9-deoxo-9a-methyl-9a-aza-9a-homoerythromycin A,
2′-O-{3-[(N,N-Diethylglycyl)amino]propyl}-9-deoxo-9a-methyl-9a-aza-9a-homoerythromycin A,
2′-O-{3-[(4-Pyridinylcarbonyl)amino]propyl}-9-deoxo-9a-methyl-9a-aza-9a-homoerythromycin A,
2′-O-{3-[(N,N-Dimethylglycyl)amino]propyl}-9-deoxo-9a-methyl-9a-aza-9a-homoerythromycin A,
2′-O-{3-[(N-Methylglycyl)amino]propyl}-9-deoxo-9a-methyl-9a-aza-9a-homoerythromycin A,
2′-O-[3-(L-prolylamino)propyl]-9-deoxo-9a-methyl-9a-aza-9a-homoerythromycin A,
2′-O-[3-(L-phenylalanylamino)propyl]-9-deoxo-9a-methyl-9a-aza-9a-homoerythromycin A,
2′-O-[3-(L-isoleucylamino)propyl]-9-deoxo-9a-methyl-9a-aza-9a-homoerythromycin A, and
2′-O-[3-(L-methionylamino)propyl]-9-deoxo-9a-methyl-9a-aza-9a-homoerythromycin A,
2′-O-(3-{[4-(Methyloxy)-4-oxobutanoyl]amino}propyl)-9-deoxo-9a-methyl-9a-aza-9a-homoerythromycin A,
2′-O-(3-{[5-(methyloxy)-5-oxopentanoyl]amino}propyl)-9-deoxo-9a-methyl-9a-aza-9a-homoerythromycin A,
2′-O-{3-[(cyclobutylcarbonyl)amino]propyl}-9-deoxo-9a-methyl-9a-aza-9a-homoerythromycin A,
2′-O-{3-[(Methoxyacetyl)amino]propyl}-9-deoxo-9a-methyl-9a-aza-9a-homoerythromycin A,
2′-O-{3-[(3-Furanylcarbonyl)amino]propyl}-9-deoxo-9a-methyl-9a-aza-9a-homoerythromycin A,
2′-O-(3-{[(5-methyl-2-pyrazinyl)carbonyl]amino}propyl)-9-deoxo-9a-methyl-9a-aza-9a-homoerythromycin A,
2′-O-(3-{[(1,3,5-trimethyl-1H-pyrazol-4-yl)carbonyl]amino}propyl)-9-deoxo-9a-methyl-9a-aza-9a-homoerythromycin A,
2′-O-{3-[(3-pyridinylcarbonyl)amino]propyl}-9-deoxo-9a-methyl-9a-aza-9a-homoerythromycin A,
2′-O-{3-[(ethyloxyacetyl)amino]propyl}-9-deoxo-9a-methyl-9a-aza-9a-homoerythromycin A,
2′-O-(3-{[3-hydroxy-2-(hydroxymethyl)-2-methylpropanoyl]amino}propyl)-9-deoxo-9a-methyl-9a-aza-9a-homoerythromycin A,
2′-O-{3-[(2-pyrazinylcarbonyl)amino]propyl}-9-deoxo-9a-methyl-9a-aza-9a-homoerythromycin A,
2′-O-(3-{[(2,5-dimethyl-3-furanyl)carbonyl]amino}propyl)-9-deoxo-9a-methyl-9a-aza-9a-homoerythromycin A,
2′-O-{3-[(tetrahydro-2H-pyran-4-ylcarbonyl)amino]propyl}-9-deoxo-9a-methyl-9a-aza-9a-homoerythromycin A,
2′-O-(3-{[(1,2,5-trimethyl-1H-pyrrol-3-yl)carbonyl]amino}propyl)-9-deoxo-9a-methyl-9a-aza-9a-homoerythromycin A,
2′-O-(3-{[(1-methyl-1H-imidazol-5-yl)carbonyl]amino}propyl)-9-deoxo-9a-methyl-9a-aza-9a-homoerythromycin A,
2′-O-[3-({[1-methyl-3-(trifluoromethyl)-1H-pyrazol-4-yl]carbonyl}amino)propyl]-9-deoxo-9a-methyl-9a-aza-9a-homoerythromycin A,
2′-O-(3-{[(4-chlorophenyl)carbonyl]amino}propyl)-9-deoxo-9a-methyl-9a-aza-9a-homoerythromycin A,
2′-O-{3-[(hydroxyacetyl)amino]propyl}-9-deoxo-9a-methyl-9a-aza-9a-homoerythromycin A,
2′-O-{3-[(2-pyridinylcarbonyl)amino]propyl}-9-deoxo-9a-methyl-9a-aza-9a-homoerythromycin A,
2′-O-{3-[(2,5-dihydroxyphenylcarbonyl)amino]propyl}-9-deoxo-9a-methyl-9a-aza-9a-homoerythromycin A,
2′-O-(3-{[(4-amino-2-hydroxyphenyl)carbonyl]amino}propyl)-9-deoxo-9a-methyl-9a-aza-9a-homoerythromycin A,
2′-O-(3-{[(2-chloro-3-pyridinyl)carbonyl]amino}propyl)-9-deoxo-9a-methyl-9a-aza-9a-homoerythromycin A,
2′-O-(3-{[(2-chloro-6-methyl-3-pyridinyl)carbonyl]amino}propyl)-9-deoxo-9a-methyl-9a-aza-9a-homoerythromycin A,
2′-O-(3-{[(5-amino-3-pyridinyl)carbonyl]amino}propyl)-9-deoxo-9a-methyl-9a-aza-9a-homoerythromycin A,
2′-O-(3-{[(3-amino-2-pyrazinyl)carbonyl]amino}propyl)-9-deoxo-9a-methyl-9a-aza-9a-homoerythromycin A,
2′-O-(3-{[(5-chloro-1,3-dimethyl-1H-pyrazol-4-yl)carbonyl]amino}propyl)-9-deoxo-9a-methyl-9a-aza-9a-homoerythromycin A,
2′-O-(3-{[(2,5-dimethyl-1,3-oxazol-4-yl)carbonyl]amino}propyl)-9-deoxo-9a-methyl-9a-aza-9a-homoerythromycin A,
2′-O-{3-[(1-methyl-L-prolyl)amino]propyl}-9-deoxo-9a-methyl-9a-aza-9a-homoerythromycin A,
2′-O-{3-[(3-methylbutanoyl)amino]propyl}-9-deoxo-9a-methyl-9a-aza-9a-homoerythromycin A,
or a salt thereof.

10. A compound of Formula (I) as claimed in claim 1 which is 2′-O-{3-[(methoxyacetyl)amino]propyl}-9-deoxo-9a-methyl-9a-aza-9a-homoerythromycin A, or a salt thereof.

11. A compound of Formula (I) as claimed in claim 1 which is 2′-O-{3-[(ethyloxyacetyl)amino]propyl}-9-deoxo-9a-methyl-9a-aza-9a-homoerythromycin A, or a salt thereof.

12. A compound of Formula (I) or a salt thereof as claimed in claim 1, wherein the salt is a pharmaceutically acceptable salt.

13. A method for the treatment of neutrophil dominated inflammatory diseases resulting from neutrophilic infiltration and/or diseases associated with altered cellular functionality of neutrophils selected from chronic obstructive pulmonary disease, cystic fibrosis, diffuse panbronchiolitis, bronchiolitis obliterans, bronchitis, bronchiectasis, adult respiratory distress syndrome, severe or steroid-resistant asthma, emphysema, chronic rhinosinusitis, rheumatoid arthritis, gouty arthritis, inflammatory bowel disease, glomerulonephritis, damage from ischemic reperfusion, atherosclerosis, dermatoses such as psoriasis and vasculitis, systemic lupus erythematosus, systemic inflammatory response syndrome, sepsis, ischemia-reperfusion injury, rosacea, periodontitis, gingival hyperplasia and prostatitis syndrome in a subject in need of such treatment comprising administering to the subject a therapeutically effective amount of a compound of Formula (I) as claimed in claim 1 or a pharmaceutically acceptable salt thereof.

14. The method of claim 13 wherein the subject in need of treatment is human.

15. The method of claim 13, wherein disease is selected from chronic obstructive pulmonary disease, cystic fibrosis, diffuse panbronchiolitis, bronchiolitis obliterans, bronchitis, bronchiectasis, acute respiratory distress syndrome, severe or steroid-resistant asthma, emphysema and chronic rhinosinusitis.

16. A pharmaceutical composition comprising a) a compound of Formula (I) as claimed in claim 1, or a pharmaceutically acceptable salt thereof and b) one or more pharmaceutically acceptable carriers.

17. A compound of Formula (I) as claimed in claim 1, or a pharmaceutically acceptable salt thereof, for use in medical therapy.

18. (canceled)

19. (canceled)

20. (canceled)

21. (canceled)

22. A combination comprising a) a compound of Formula (I) as claimed in claim 1, or a pharmaceutically acceptable salt thereof and b) one or more further therapeutically active agents.
